(19) United States
(12) Patent Application Publication
(10) Pub. No.: US 2026/0015642 A1
(43) Pub. Date: Jan. 15, 2026

(54) HIGH-EFFICIENCY RECONSTITUTION OF RNA MOLECULES

(71) Applicant: Salk Institute for Biological Studies, La Jolla, CA (US)

(72) Inventors: Lukas Christoph Bachmann, San Diego, CA (US); Samuel Lawrence Pfaff, San Diego, CA (US)

(73) Assignee: Salk Institute for Biological Studies, La Jolla, CA (US)

(21) Appl. No.: 19/331,661

(22) Filed: Sep. 17, 2025

Related U.S. Application Data
(63) Continuation of application No. 17/486,488, filed on Sep. 27, 2021, now Pat. No. 12,448,636.
Application No. 17/486,488 is a continuation of application No. PCT/US2020/025430, filed on Mar. 27, 2020.
(60) Provisional application No. 62/826,854, filed on Mar. 29, 2019.
(60) Provisional application No. 62/834,305, filed on Apr. 15, 2019.
(60) Provisional application No. 62/888,855, filed on Aug. 19, 2019.
(60) Provisional application No. 62/933,714, filed on Nov. 11, 2019.

(51) Int. Cl. C12P 19/34; C12N 15/86; C12Q 1/6813

(52) U.S. Cl. CPC C12P 19/34; C12N 15/86; C12Q 1/6813; C12N 2750/14111

(57) ABSTRACT

Provided herein are synthetic RNA molecules for reconstitution of RNA molecules, including compositions and methods of using these molecules. For example, such molecules can be used to deliver a protein coding sequence over two or more viral vectors (such as AAVs), resulting in reconstitution of the full-length protein in a cell. Such methods can be used to deliver a therapeutic protein, for example to treat a genetic disease or cancer.

### CROSS-REFERENCE TO RELATED APPLICATIONS

This is a continuation of U.S. patent application Ser. No. 17/486,488, filed Sep. 27, 2021, which is a continuation of PCT Application No. PCT/US2020/025430, filed March 27 2020, which claims priority to U.S. Provisional Application No. 62/826,854 filed Mar. 29, 2019, U.S. Provisional Application No. 62/834,305 filed Apr. 15, 2019, U.S. Provisional Application No. 62/888,855 filed Aug. 19, 2019, and U.S. Provisional Application No. 62/933,714 filed Nov. 11, 2019, all herein incorporated by reference.

### REFERENCE TO AN ELECTRONIC SEQUENCE LISTING

The present application is filed along with a sequence listing in electronic format. The sequence listing is provided as a file entitled SequenceListing.xml, created on Sep. 2, 2025, which is approximately 229,883 bytes in size. The information in the electronic format of the sequence listing is incorporated herein by reference in its entirety.

### FIELD

The present disclosure provides systems, kits, compositions, and methods that allow for reconstitution of two or more RNA molecules, allowing expression of a full-length protein.

### BACKGROUND

Several hereditary diseases are caused by recessive loss of function mutations in a single gene. In such cases, gene replacement therapy (or gene therapy) is a promising treatment strategy. Adeno-associated virus (AAV) is a preferred vector for gene replacement therapy, but treatment of several diseases has remained challenging due to the incompatibility of large size of disease-linked genes with the limited packaging capacity of AAV (or other gene therapy vectors). For example, the genome-packaging capacity of AAV is about 5000 nucleotides. Even if the replacement gene is within the cargo capacity of the gene therapy vector, lack of space for adequate regulatory sequences can prevent efficient expression in a desired tissue.

Strategies to overcome the packaging constraints of gene therapy vectors have been explored in the past, but efficiencies of such attempts have remained low which highlights the need for further clinical methods.

### SUMMARY

Provided herein are systems for expressing a target protein. In one example, the system includes (1) a first synthetic nucleic acid molecule, comprising from 5′ to 3′, a first promoter; an RNA molecule encoding an N-terminal portion of the target protein operably linked to the first promoter, which includes a first splice junction at a 3′-end of the RNA molecule encoding the N-terminal portion of the target protein; a splice donor; and a first dimerization domain; and (2) a second synthetic nucleic acid molecule; comprising from 5′ to 3′, a second promoter; a second dimerization domain operably linked to the second promoter, and having reverse complementarity to the first dimerization domain; a branch point sequence; a polypyrimidine tract; a splice acceptor; and an RNA molecule encoding a C-terminal portion of a target protein, which includes a splice junction at a 5′-end of the RNA molecule encoding the C-terminal portion of a target protein.

In one example, the system includes (1) a first synthetic nucleic acid molecule, comprising from 5′ to 3′, a first promoter, an RNA molecule encoding an N-terminal portion of the target protein operably linked to the first promoter, which includes a splice junction at a 3′-end of the RNA molecule encoding the N-terminal portion of the target protein; a first splice donor; and a first dimerization domain; (2) a second synthetic nucleic acid molecule, comprising from 5′ to 3′, a second promoter; a second dimerization domain operably linked to the second promoter, and having reverse complementarity to the first dimerization domain; a first branch point sequence; a first polypyrimidine tract; a first splice acceptor; an RNA molecule encoding a middle portion of a target protein, which includes a splice junction at a 5′-end of the RNA molecule encoding the middle portion of a target protein and a splice junction at a 3′-end of the RNA molecule encoding the middle portion of the target protein; a second splice donor; and a third dimerization domain; and (3) a third synthetic nucleic acid molecule; comprising from 5′ to 3′, a third promoter, a fourth dimerization domain operably linked to the third promoter, and having reverse complementarity to the third dimerization domain; a second branch point sequence, a second polypyrimidine tract, a second splice acceptor; and an RNA molecule encoding a C-terminal portion of a target protein, which includes a splice junction at a 5′-end of the RNA molecule encoding the C-terminal portion of a target protein.

In some examples, the synthetic nucleic acid molecules include one or more splicing enhancers.

In some examples, the synthetic nucleic acid molecules are part of a vector, such as a viral vector, such as AAV or a lentiviral vector.

Also provided are compositions and kits that include the disclosed systems.

Also provided are methods of using the disclosed systems to express a protein in a cell. Such a method can include introducing the system into a cell, and expressing the synthetic first and second, first, second, and third, or first, second, third and fourth nucleic acid molecules in the same cell. In some examples, the cell is a subject, and the method treats a disease in the subject, such as a genetic disease caused by a mutation in a gene encoding the target protein, or treats cancer in the subject (wherein the target protein is a toxin or thymidine kinase). In some examples, administration is via injection, such as iv.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts a schematic of vector designs.

FIG. 1B depicts transfection of only the N-terminal expression plasmid does not lead to YFP fluorescence.

FIG. 1C depicts transfection of only the C-terminal expression plasmid does not lead to YFP fluorescence.

FIG. 1D depicts expression of N-terminal and C-terminal fragments without binding domains shows low levels of YFP induction.

FIG. 1E depicts rationally designed dimerization/binding domain in a looped configuration.

FIG. 1F depicts 3D rendering of the “looped” dimerization domain configuration.

FIG. 1G depicts negative control with no binding domain on the C-terminal half.

FIG. 1H depicts negative control with no binding domain on the N-terminal half.

FIG. 1I depicts matching binding domains on both N- and C-terminal half shows strong YFP induction in 90% of the cells.

FIGS. 1J-1N depict data equivalent to that in FIGS. 1E-1I for a configuration of a binding domain with a stretch of 150 hypodiverse exclusively pyrimidine or exclusively purine containing sequence resulting in a fully open configuration.

FIG. 1O depicts representative fluorescence images for cells shown in FIG. 1G.

FIG. 1P depicts representative fluorescence images for cells shown in FIG. 1L.

FIG. 1Q depicts a comparison of conditions shown in FIG. 1D, FIGS. 1G-1I, and FIGS. 1L-1N.

FIG. 2A depicts schematic of vector designs. The protein coding sequence of a yellow fluorescent protein (YFP) is split into an N-terminal, a middle fragment (m-yfp) and a C-terminal fragment. The junction of the n and m fragments is joined by a looped design binding domain (BD1) and the junction between m and c fragments is joined by a looped binding domain (BD2). The pyrimidina (Y) and purine (R) sequences are arranged in such a way as to avoid self-circularization of the m-fragment and avoid direct recombination of the N- and C-fragment. The N-terminal fragment is co-expressed with red fluorescent protein as a transfection control, the C-terminal fragment is coexpressed with blue fluorescent protein as a transfection control.

FIG. 2B depicts matching binding domains on all three fragments shows strong YFP induction in 80% of the cells. Flow cytometry displaying red and green fluorescence values for 20 k BFP+ cells.

FIG. 2C depicts representative fluorescent image of expression of the n and m fragment only shows no yfp fluorescence (negative control).

FIG. 2D depicts representative fluorescent image of expression of the m and c fragment only shows no yfp fluorescence (negative control).

FIG. 2E depicts representative fluorescent image showing that strong YFP fluorescence is induced by co-transfection of all three fragments.

FIGS. 3A-3D depict efficient reconstitution of yellow fluorescent protein (YFP) from two fragments (SEQ ID NOS: 1 and 2) expressed from two AAV2/8s after systemic administration in the newborn (P3) mouse pup. (A) depicts one RNA encoding the n-terminal half fragment of YFP, and one RNA encoding the c-terminal half fragment, which are coexpressed using AAV. (B) depicts native YFP fluorescence in the liver of the juvenile mouse at the time of sacrifice (green). Uninjected liver is shown for comparison. DRAQ5 nuclear stain is shown in magenta for context. (C) depicts strong native YFP fluorescence in the heart muscle at the time of sacrifice (green). Top panels show macroscopic view and red autofluorescence for context (in magenta). Bottom panel shows cross-section with DRAQ5 nuclear stain for context (in magenta). Uninjected mouse heart is shown for control. (D) depicts strong native YFP fluorescence in the skeletal muscles of the leg at the time of sacrifice. Uninjected mouse legs are shown for comparison. Top panels show macroscopic view with red autofluorescence in magenta. Bottom panel shows microscopic image of a cross-section through the leg. Bottom panel shows DRAQ5 nuclear stain in magenta for context.

FIGS. 4A-4B depict efficient reconstitution of yellow fluorescent protein (YFP) from three fragments (SEQ ID NOS: 145, 146 and 2, respectivley) in the mouse tibialis anterior muscle after intramuscular injection of three AAV2/8 in the newborn (P3) mouse pup. (A) depicts a schematic of three AAV particles encoding a full-length YFP that is split into three fragments. (B) Shows strong native YFP fluorescence in a longitudinal section of the tibialis anterior muscle of a mouse injected with all three viral particles. DRAQ5 nuclear stain is shown in magenta for context.

FIGS. 5A-5F depict efficient reconstitution of yellow fluorescent protein (YFP) from two and from three fragments in adult mouse tibialis anterior muscle. (A) depicts N-terminal and C-terminal halves of YFP coding sequence are equipped with synthetic RNA-dimerization and recombination domains. (B) depicts two AAV transfer plasmids expressing these two fragments were electroporated transcutaneously into adult mouse tibialis anterior (TA) muscle and strong fluorescence was detected at 5 days post electroporation. (C) depicts no fluorescence was detectable in contralateral non-injected TA. (D) depicts n-terminal, middle, and c-terminal YFP coding sequence are equipped with synthetic RNA-dimerization and recombination domains linking each fragment to its adjacent fragment(s). (E) depicts transcutaneous electroporation of three AAV transfer plasmids expressing these three fragments. Strong YFP fluorescence is detected indicating efficient reconstitution of YFP from three fragments. (F) depicts fluorescence in contralateral non-injected TA. Fluorescent channel is overlaid onto grey scale photographs for context.

FIG. 6A is a schematic drawing providing an exemplary system for the disclosed RNA recombination methods, using two nucleic acid molecules 110, 150, wherein the target protein is divided into two portions and each portion is encoded by a different nucleic acid molecule. Drawing not to scale.

FIG. 6B is a schematic drawing providing an exemplary dimerization domain (e.g., 122, 154 of FIG. 6A) that includes hypodiverse sequences interspersed with sequences that can form a stem, which results in local RNA loops that are open and available for basepairing in the absence of pseudoknot formation. Drawing not to scale.

FIG. 6C is a schematic drawing showing the interaction and hybridization (base pairing) between dimerization domain 122 of molecule 110 (FIG. 6A) and dimerization domain 154 of molecule 150 (FIG. 6A), allows the spliceosome components to recombine N-terminal coding sequence 114 and C-terminal coding sequence 164. The results in the 3′ end of the N terminal protein coding sequence 114 fusing to the 5′ end of the C terminal protein sequence 164, and a seamless junction between the N- and C-terminal portions.

FIG. 6D is a schematic drawing providing an exemplary system for the disclosed RNA recombination methods, using three nucleic acid molecules 110, 200, 150, wherein the target protein is divided into three portions (N-terminal, middle, C-terminal) and each portion is encoded by a different nucleic acid molecule. Drawing not to scale.

FIG. 6E is a schematic drawing showing the interaction and hybridization (base pairing) between dimerization domain 122 of molecule 110 (FIG. 6D) and dimerization domain 204 of molecule 200 (FIG. 6D), and between dimerization domain 226 of molecule 200 (FIG. 6D) and dimerization domain 154 of molecule 150 (FIG. 6D), allows the spliceosome components to recombine N-terminal coding sequence 114, middle coding sequence 216, and C-terminal coding sequence 164. The results in the 3′ end of the N terminal coding sequence 114 fusing to the 5′ end of the middle protein sequence 216, and the 3′ end of the middle coding sequence 216 fusing to the 5′ end of the C-terminal sequence 216, and a seamless junction between the N-, middle, and C-terminal portions.

FIG. 7A is a schematic drawing providing an exemplary system for the disclosed RNA recombination methods, that like FIG. 6A uses two nucleic acid molecules 500, 600, but the dimerization domains are aptamers 512, 602, that recognize the same target molecule 700. Drawing not to scale.

FIG. 7B is a schematic drawing providing an exemplary system for the disclosed RNA recombination methods, that, related to FIG. 7A, uses dimerization domains that recognize the same target molecule. Here, the target recognized by the dimerization domain is a specific RNA molecule (instead of molecule 700 in FIG. 7A, e.g., protein or small molecule). Each domain recognizes a different portion of an mRNA molecule only expressed in target cells (i.e., cells where target protein expression is desired), such as a cancer-specific transcript. Drawing not to scale.

FIG. 7C is a schematic drawing providing an exemplary system for the disclosed RNA recombination methods, that like FIG. 6A and 7A, uses two nucleic acid molecules 800, 900, and shows the dimerization domains 812, 902 hybridizing to an oligonucleotide 1000 that prevents the dimerization domains from interacting with one another, and therefore prevents or reduces recombination of the N-terminal coding sequence 802 and C-terminal coding sequence 914. Drawing not to scale.

FIG. 8 is a bar graph comparing reconstitution of YFP protein expression in the presence (w/) or absence (w/o) of a WPRE3 sequence in the 3′ untranslated region. N=3 replicates per sample are shown.

FIG. 9A is a schematic drawing providing an example for the use of dimerization domain (e.g., 122, 154 of FIG. 6A) that includes kissing loop interaction for high affinity dimerization. Using the teachings provided herein, one will appreciate that any of the disclosed coding portions (e.g., YFP) can be replaced with other target protein coding sequences.

FIG. 9B depicts RFP, BFP, and YFP signal in HEK293T cells transfected with both halves of the split YFP. Equipped with either a linear dimerization adhering to the hypodiverse design principle or a structured dimerization domain designed for kissing loop-loop interactions. Strong yellow fluorescent signal indicates efficient reconstitution.

FIGS. 10A-10Z are exemplary synthetic nucleic acid molecules that can be used with the systems and methods. In some examples, a synthetic nucleic acid molecule as at least 80%, at least 85%, at least 90%, at least 95%, at least 98%, at last 99% or 100% sequence identity to the sequence of any one of SEQ ID NOS: 1 (FIGS. 10A-10B), 2 (FIGS. 10C-10E), 7 (FIG. 10E), 8 (FIG. 10F), 9 (FIG. 10G), 10 (FIG. 10H), 11 (FIG. 10I), 12 (FIG. 10J), 13 (FIG. 10K), 14 (FIG. 10L), 15 (FIG. 10M), 16 (FIG. 10N), 17 (FIG. 10O), 18 (FIG. 10P), 19 (FIG. 10Q), 20 (FIGS. 10R-10U), and 21 (FIGS. 10V-10Z), but with a different target protein coding sequence. Thus an intronic region using with any of the systems or methods provided herein can have at least 80%, at least 85%, at least 90%, at least 95%, at least 98%, at last 99% or 100% sequence identity to any intronic sequence of SEQ ID NOS: 1, 2, 3, 4, 7, 8, 9, 10, 11, 12, 13, 14, 15, 16, 17, 18, 19, 20, or 21. For example, FIGS. 10A-D show exemplary (A,B) first (SEQ ID NO: 1) and (C,D) second (SEQ ID NO: 2) synthetic molecules that can be used to express full-length YFP, while SEQ ID NO: 3 and 4 provide the corresponding synthetic intron portion without the YFP coding portion. In some examples, a synthetic intron sequence has at least 80%, at least 85%, at least 90%, at least 95%, at least 98%, at last 99% or 100% sequence identity to SEQ ID NO: 3 or 4. Thus, the coding sequence portion of any synthetic molecule provided herein (e.g., nt 544 to 1032 of SEQ ID NO: 1 and nt 905 to 1141 of SEQ ID NO: 2), can be replaced with another coding sequence portion.

FIG. 11 is a bar graph showing the reconstitution efficiency of different length random complimentary binding domains (50 bp, 100 bp, 150 bp, 200 bp, 300 bp, 400 bp, and 500 bp). YFP median fluorescence intensity is compared between cells with matching RFP and BFP transfection levels. n=3 samples per condition. n=3 samples per condition.

FIGS. 12A-12B show that inclusion of a splice enhancer into the synthetic intron increases the reconstitution efficiency. FIG. 12A is a schematic drawing of the 5′-N and 3′-C-terminal constructs used (SEQ ID NO: 1 and 2). FIG. 12B is a bar graph showing the resulting YFP fluorescence following transfection of SEQ ID NO: 1 and 2 into cells, or various truncations thereof. n=3 samples per condition. FIGS. 13A-13D shows dual projection tracing by reconstitution of full-length flp recombinase (Flpo) from two fragments (SEQ ID NOS: 147 and 148). (A) Schematic representation of the 5′- and 3′-sequences used to reconstitute flpo. (B) Schematic representation of a mouse injected with the 5′-and 3′-sequences in different regions of the brain. (C and D) show cells with dual projections to both primary motor cortices in red. Hoechst staining (nuclei) is shown for context.

FIGS. 14A-14D show expression of oversized cargo in cell culture and in vivo in the mouse primary motor cortex. (A) Schematic representation of the 5′- and 3′-sequences used to reconstitute YFP, which include long stuffer sequences (uninterrupted open reading frames; SEQ ID NOS: 22 and 23, respectively). (B) Quantitative real-time PCR analysis of reconstitution efficiency of the oversize YFP constructs in HEK 293t cells. N=3 per condition. (C) Reconstituted YFP protein expression from full-length oversized YFP expression and split-REJ expression assessed by flow cytometry of transiently transfected HEK 293t cells. Median yellow fluorescence intensity is compared between cell populations with equal transfection control (blue and red) fluorescence for the different conditions. Y-axis shows median yellow fluorescence intensity [a.u.]. N=3 per condition. (D) Schematic of injections into mouse primary motor cortex, and images of brain tissue 10 days following injection, showing successful reconstitution of a long (2401 aa) YFP protein in vivo.

FIGS. 15A-15C show efficient reconstitution of full-length human coagulation factor VIII (FVIII) with N-terminal HA tag (substituting the N-terminal signal peptide) (2317 aa). (A) Schematic representation of the 5′- and 3′-sequences used to reconstitute FVIII (SEQ ID NOS: 24 and 25, respectively). (B) PCR amplification of the junction. (C) Western blot showing expression of FVIII. Lanes 1-3: expression of full-length FVIII (290 kDa band shows full length, unprocessed FVIII). Lanes 4-6: expression of reconstituted FVIII (band at 290 kDa shows successfully reconstituted FVIII). Lanes 7 and 8: expression of the N-terminus only shows absence of full-length FVIII band at 290 kDa. For all lanes: Expected proteolytic processing products are observed ranging from ˜75 kDa to ˜210 kDa. FVIII is probed for using a mouse anti-HA primary antibody. All lanes were loaded with 5 micrograms of cleared cell protein extract. GAPDH (rabbit anti-GAPDH) is probed for as loading control.

FIGS. 16A-16F show efficient reconstitution of full-length human Abca4 with C-terminal FLAG-tag (2300 aa). (A) Schematic representation of the 5′- and 3′-sequences used to reconstitute Abca4 (SEQ ID NOS: 20 and 21, respectively), and a Sanger sequencing trace across the junction. (B) PCR amplification of the junction. (C) Schematic representation of the probes used to assay recombination of the 5′- and 3′-fragments. (D) PCR quantification of reconstitution efficiency after two days of expression in HEK 293t cells. N=2 per condition. (E) Western blot showing expression of Abca4. Lanes 1-3: expression of full-length Abca4 (˜260 kDa band shows full length Abca4). Lanes 4-6: expression of reconstituted Abca4 (band at 260 kDa shows successfully reconstituted Abca4). Lanes 7 and 8: no transfection control (i.e., HEK 293t lysate only) shows absence of any signal. Abca4 is probed for using a mouse anti-HA primary antibody. All lanes were loaded with 5 micrograms of cleared cell protein extract. GAPDH (rabbit anti-GAPDH) is probed for as loading control. (F) Quantification of the western blot in (E) normalized for differential BFP concentration. Data is shown as normalized to the average of full-length expression control.

FIGS. 17A and 17B provide (A) HIV-1 based kissing loop dimerization domain (N-fragment, SEQ ID NO: 139, C-fragment SEQ ID NO: 140); and (B) HIV-2 based kissing loop dimerization domain (N-fragment, SEQ ID NO: 141, C-fragment SEQ ID NO: 142).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NOS: 1 and 2 are N- and C-terminal sequences, respectively, used to express full-length YFP. SEQ ID NO: 1, CMV promoter nt 1 to 543, YFP coding sequence nt 544 to 1032, synthetic intron nt 1033 to 1436, and untranslated poly A region nt 1437 to 1491. SEQ ID NO: 2,CMV promoter nt 1 to 522, synthetic intron nt 523 to 904, YFP coding sequence nt 905 to 1141, and nt 1142 to 1302 is the untranslated poly A region.

SEQ ID NOS: 3 and 4 are 5′- and 3′-intronic sequences, respectively, that can be used to express a desired full-length protein, wherein a N-terminal portion of the full-length protein can be added at nt 1 of SEQ ID NO: 3, and C-terminal portion of the full-length protein can be added at nt 382 of SEQ ID NO: 4.

SEQ ID NOS: 5 and 6 are N- and C-terminal coding sequences, respectively, used to express full-length YFP.

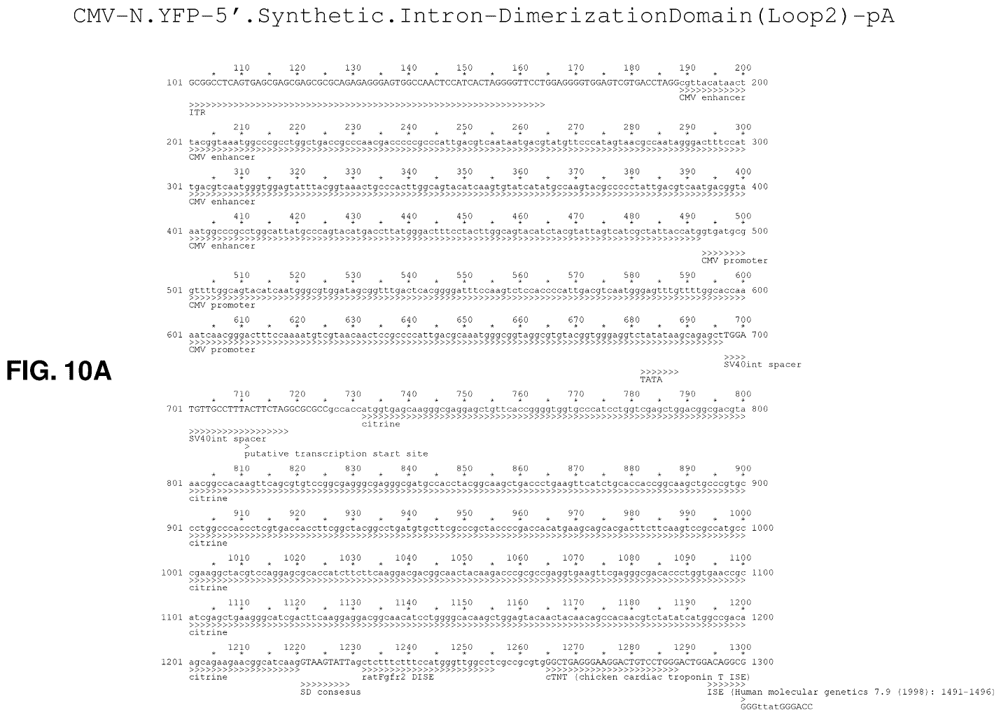

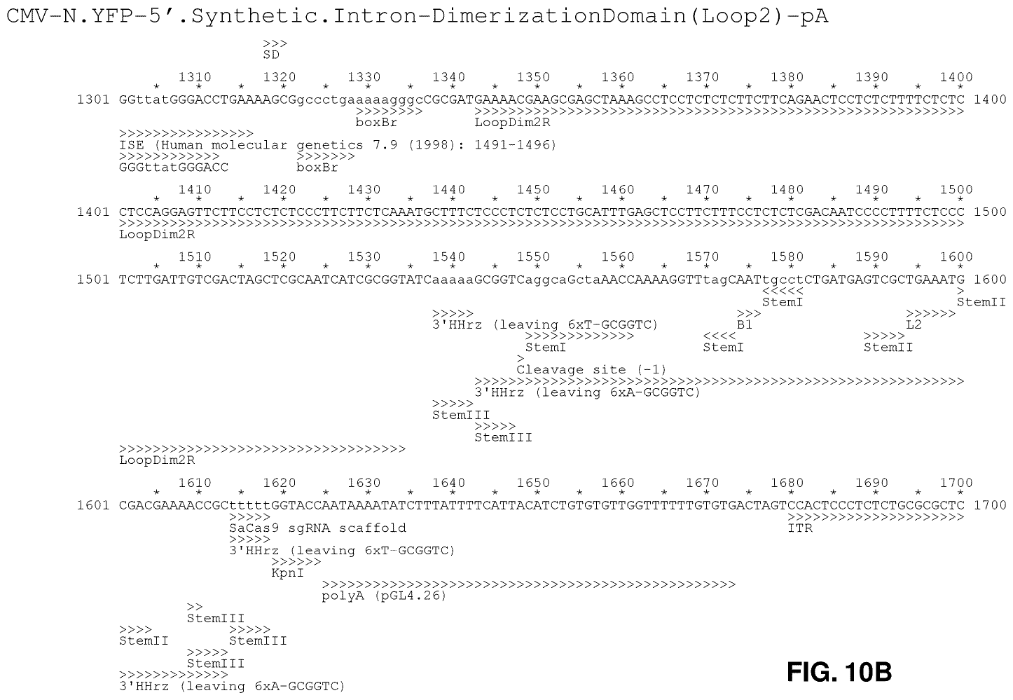

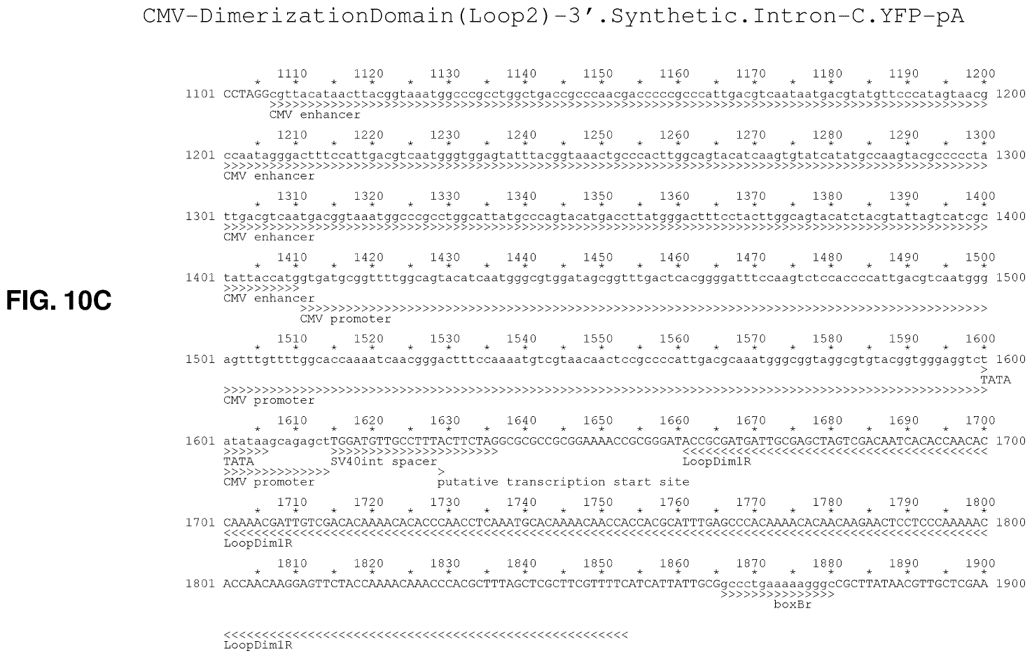

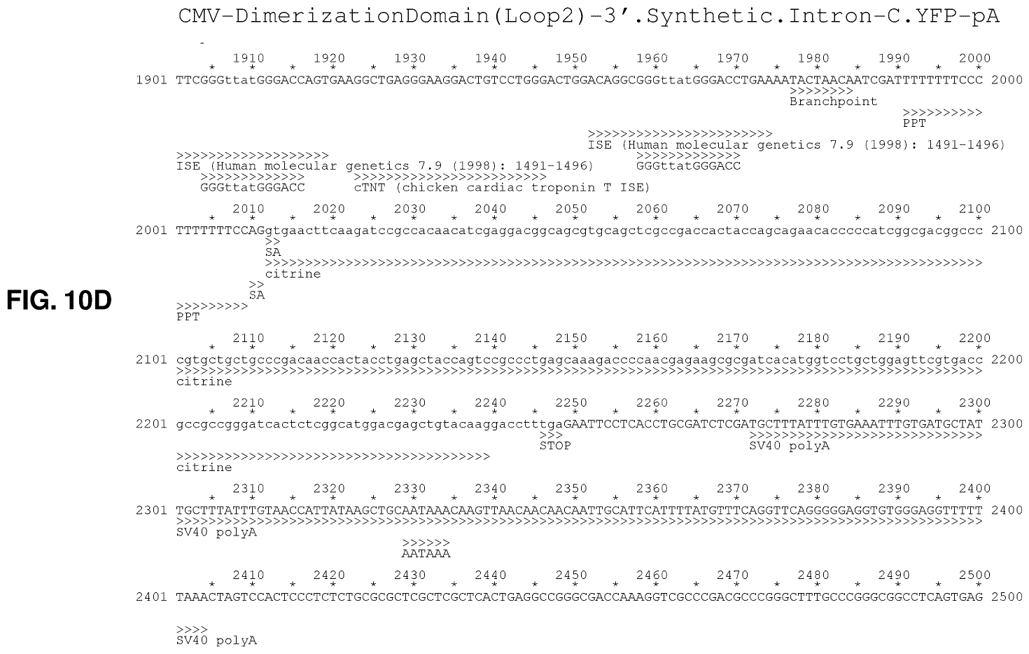

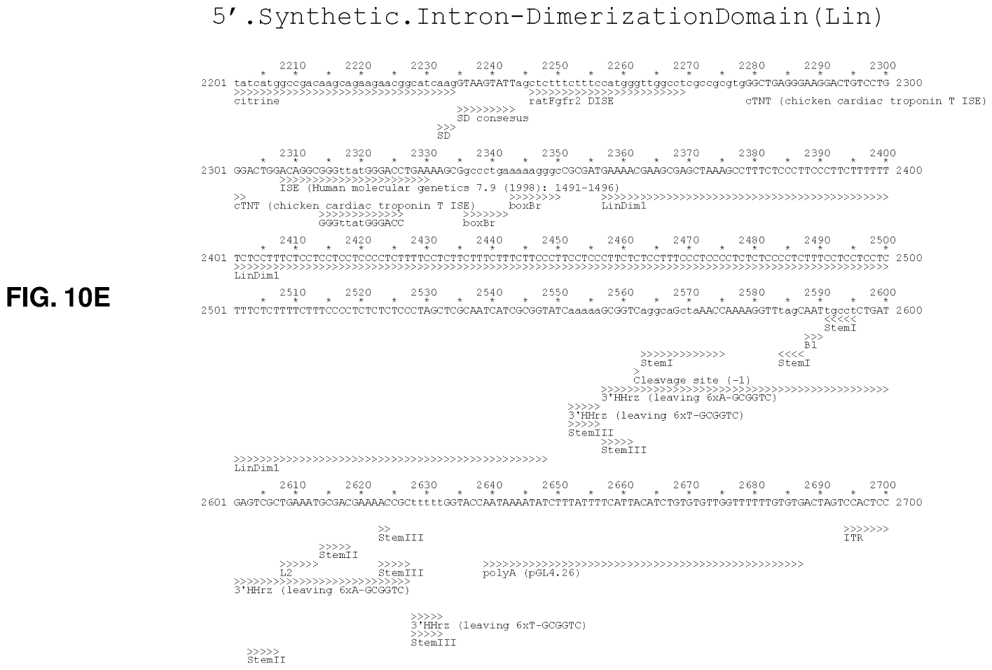

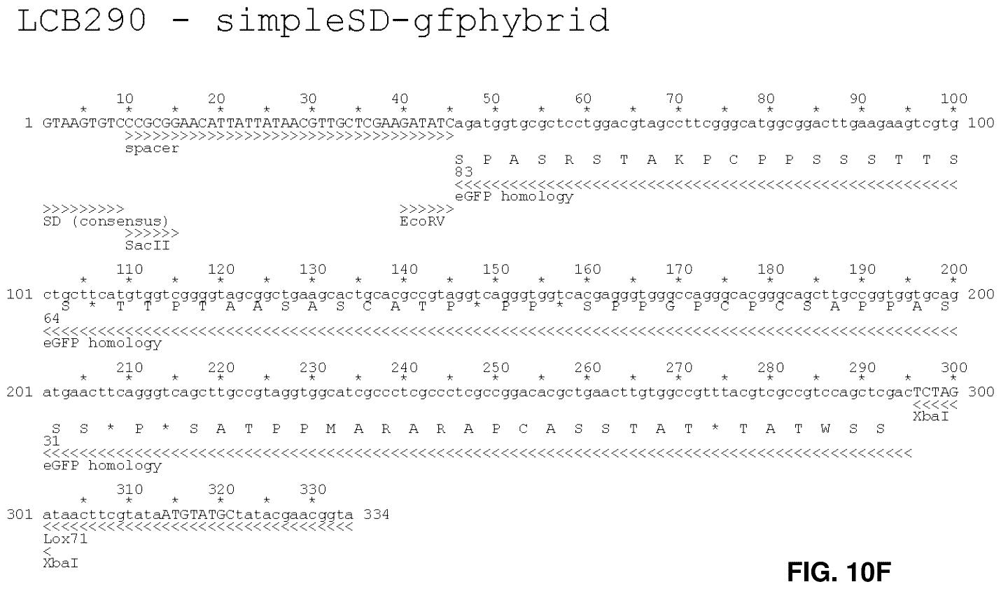

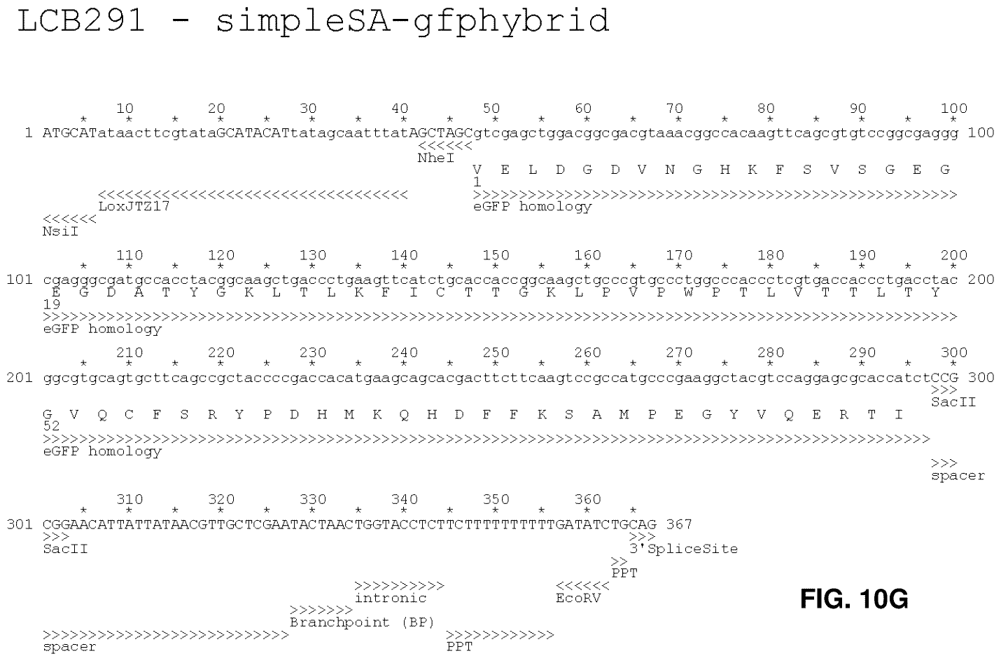

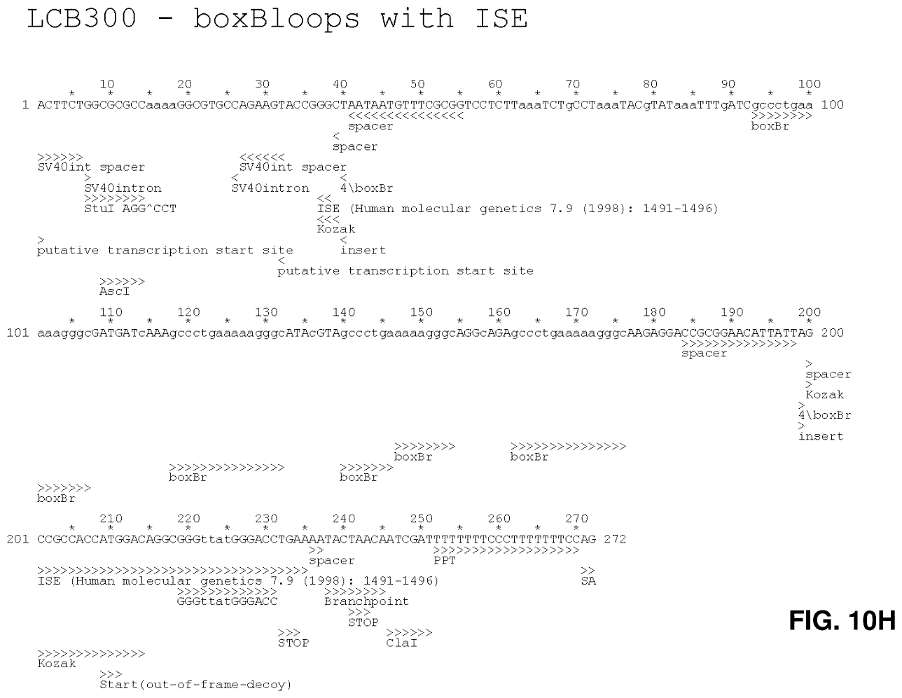

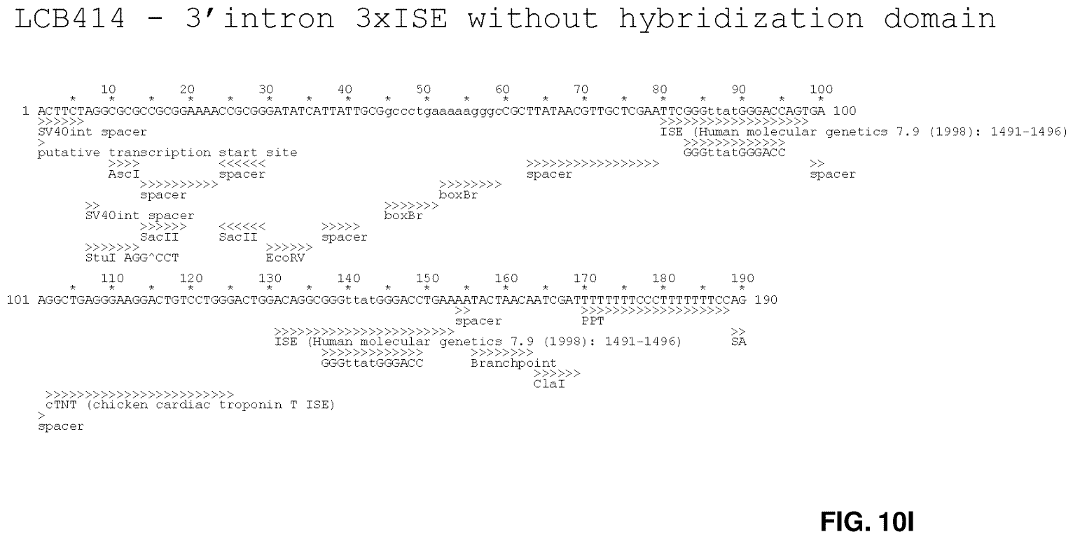

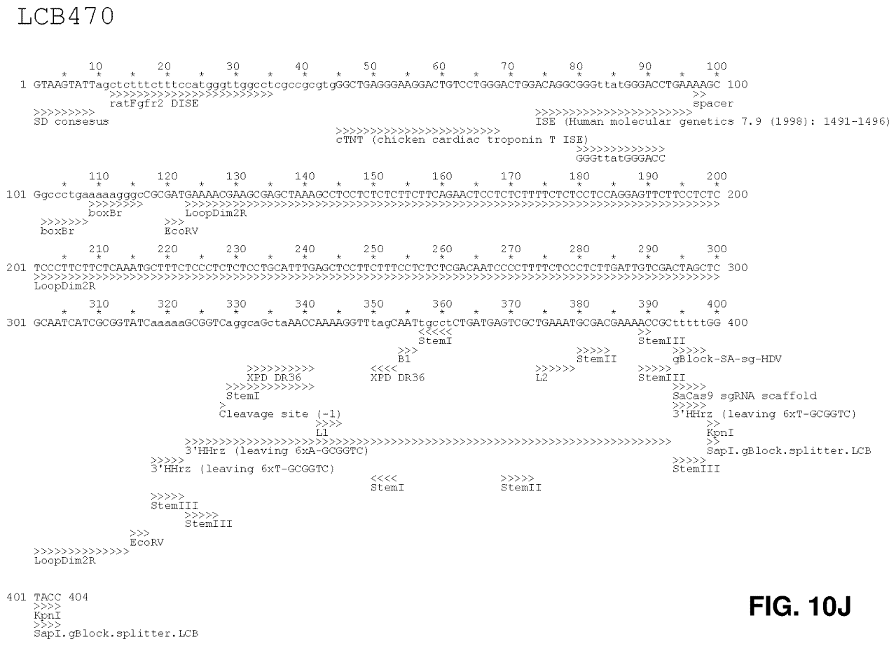

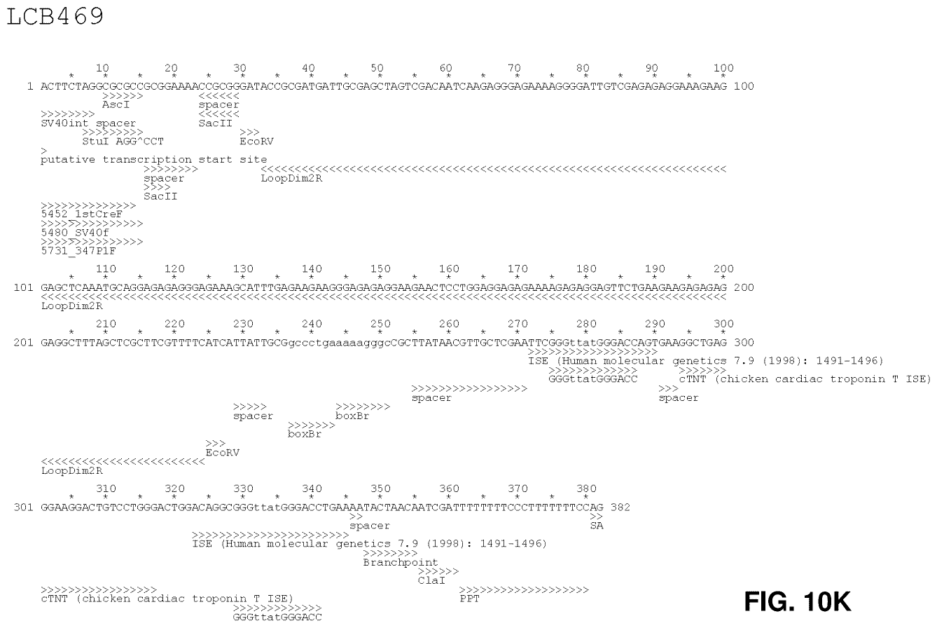

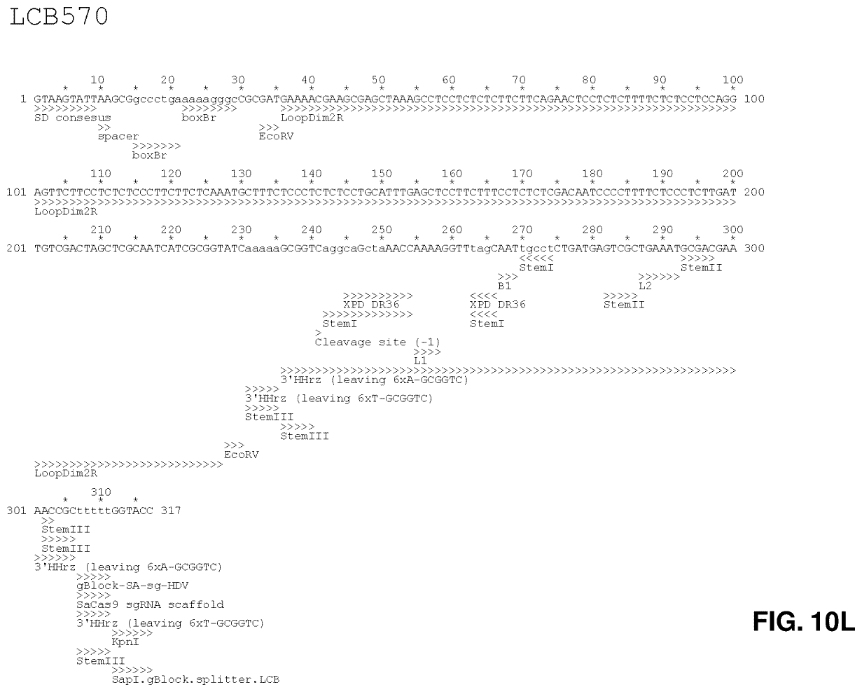

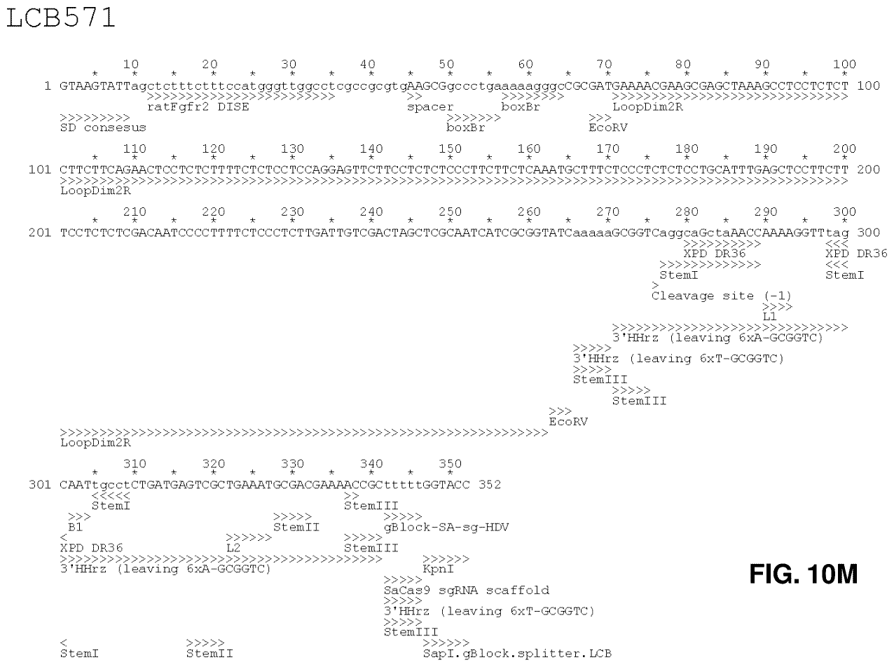

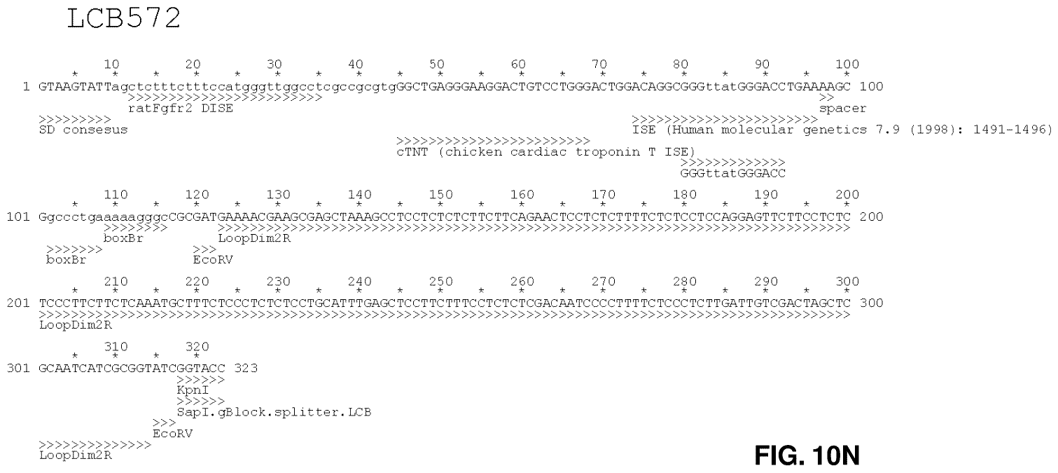

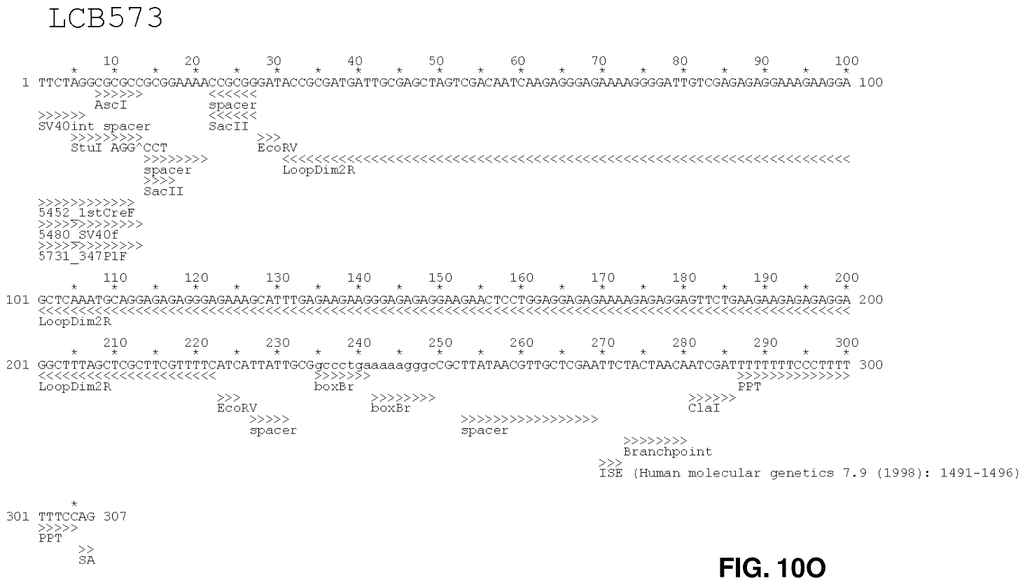

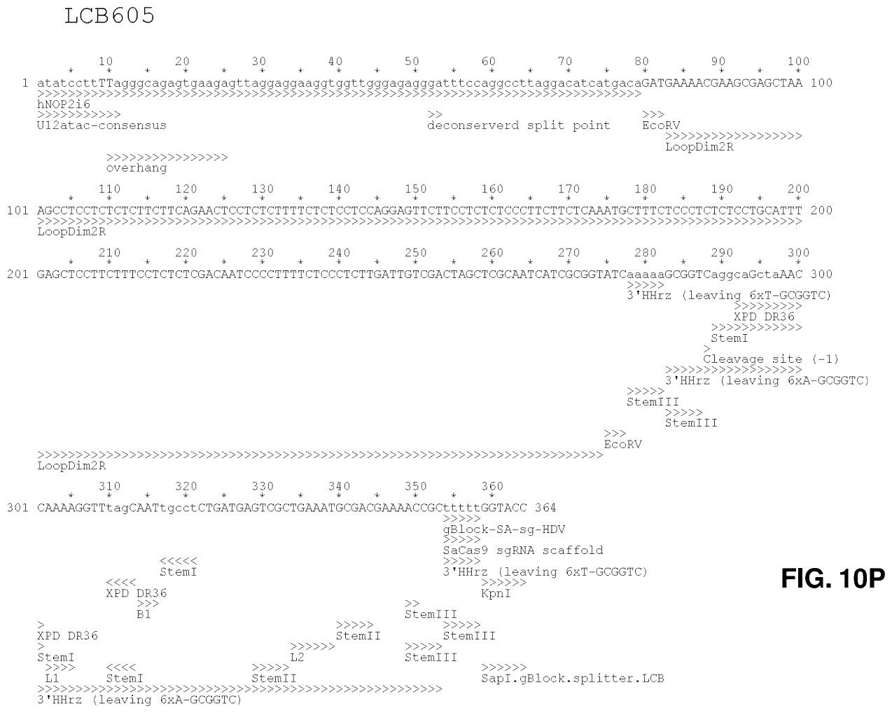

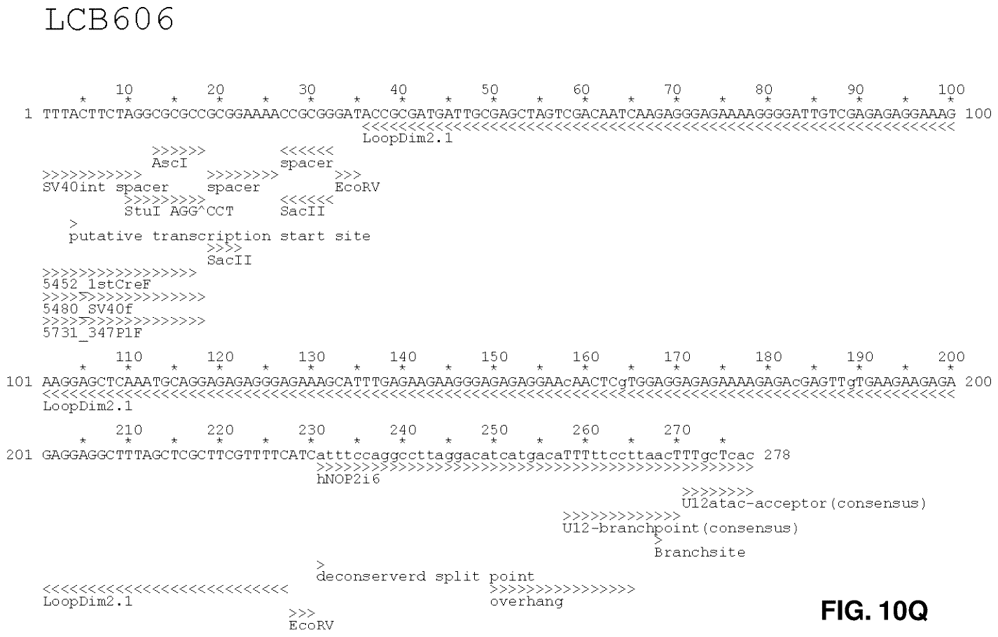

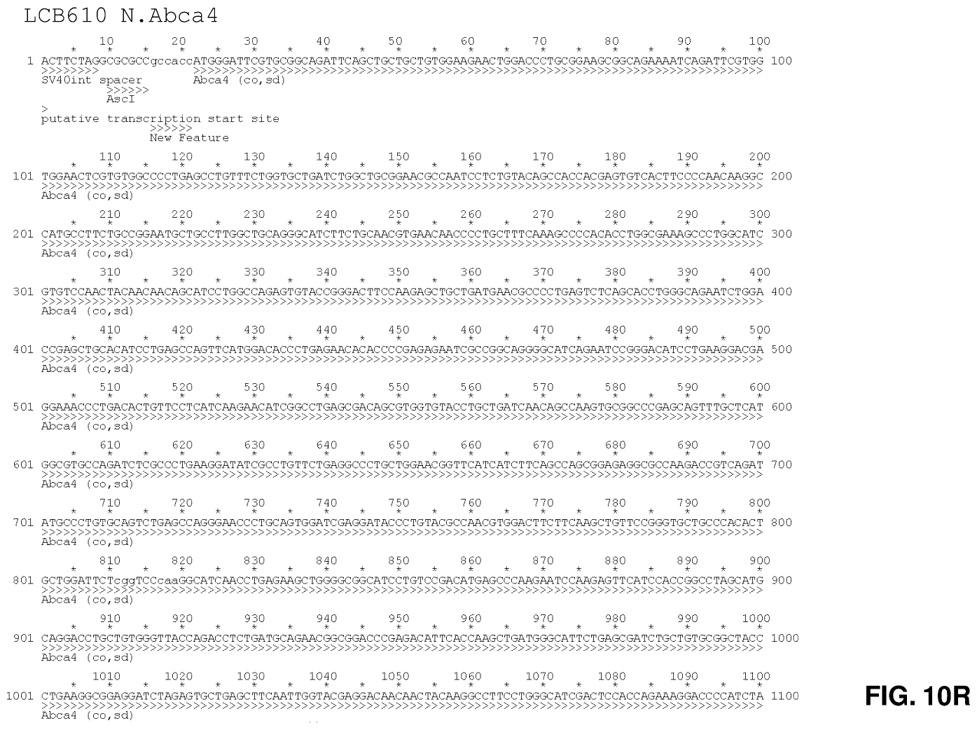

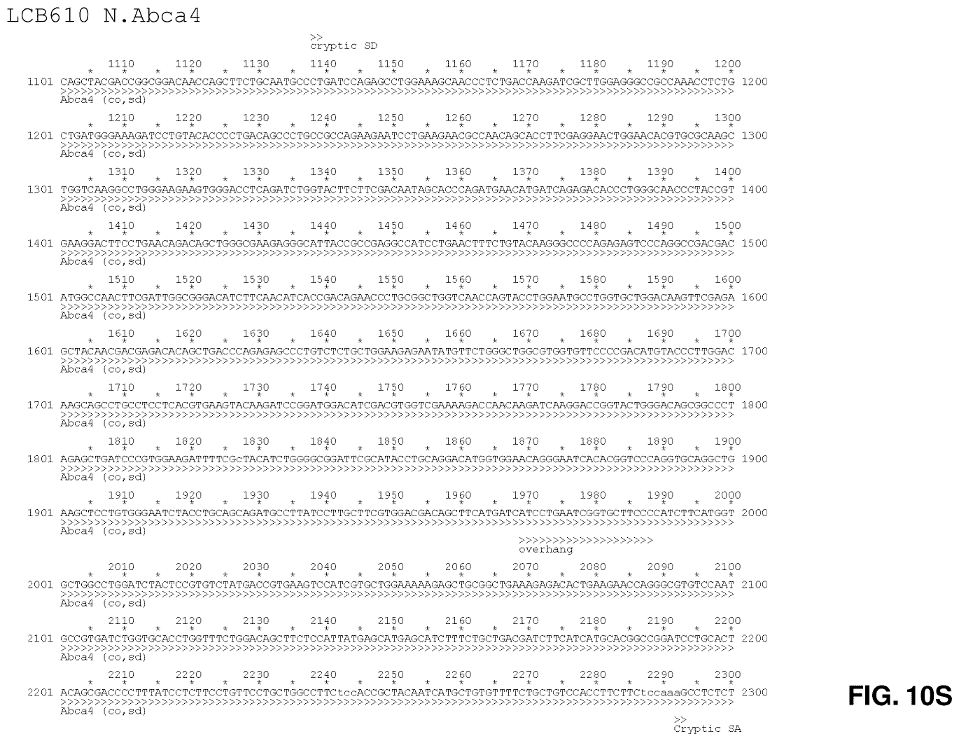

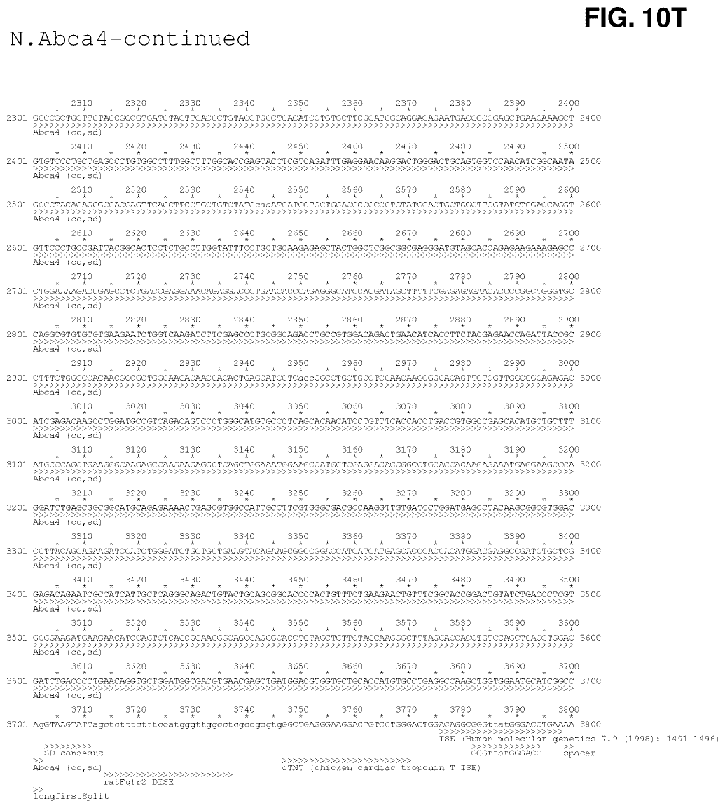

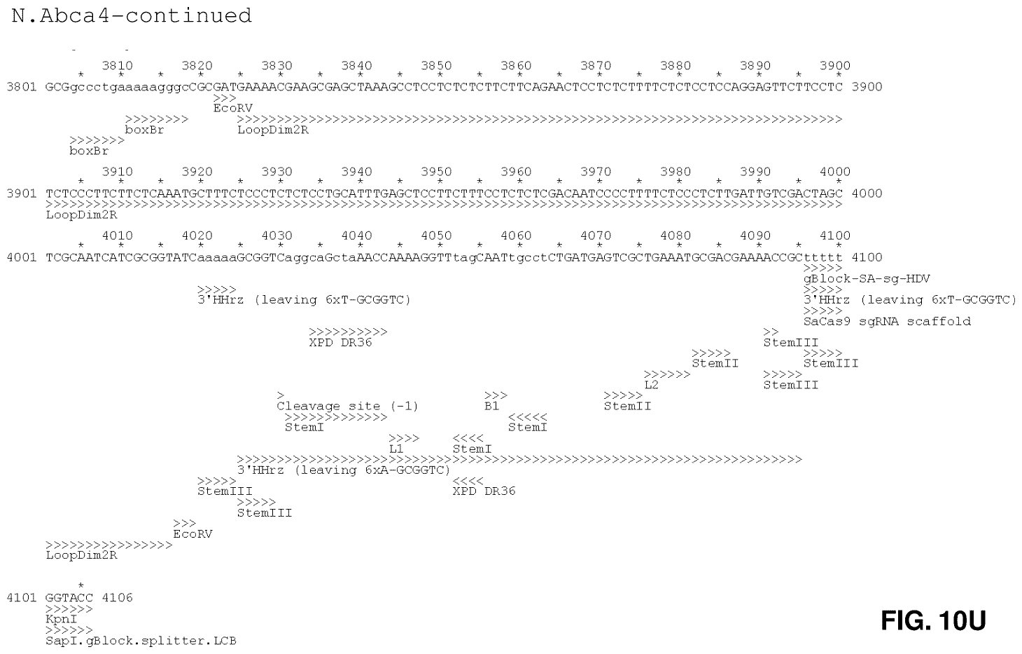

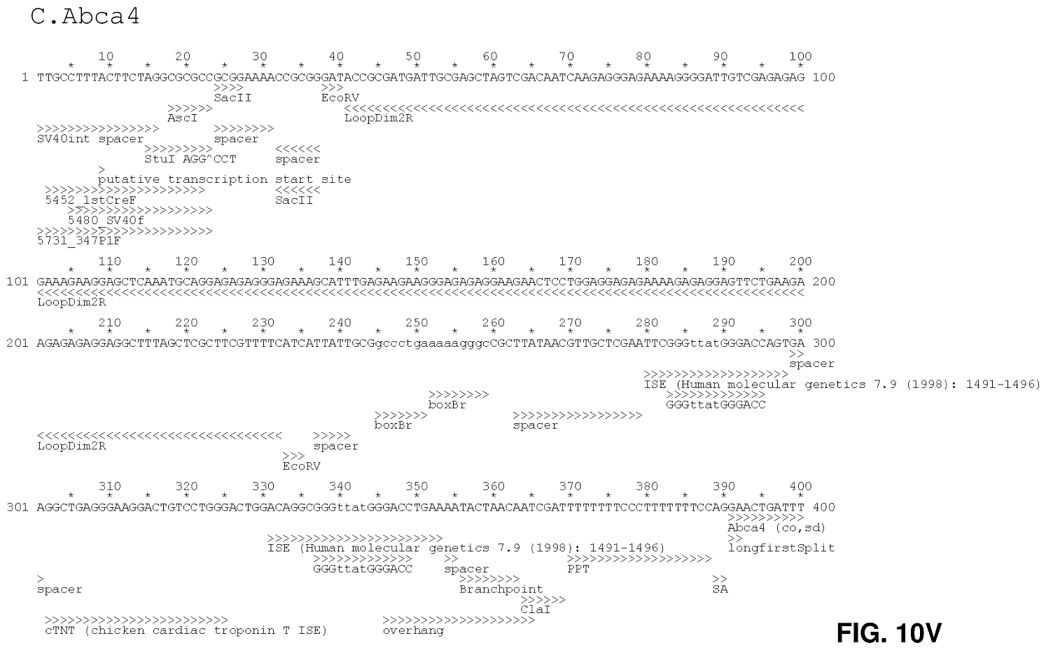

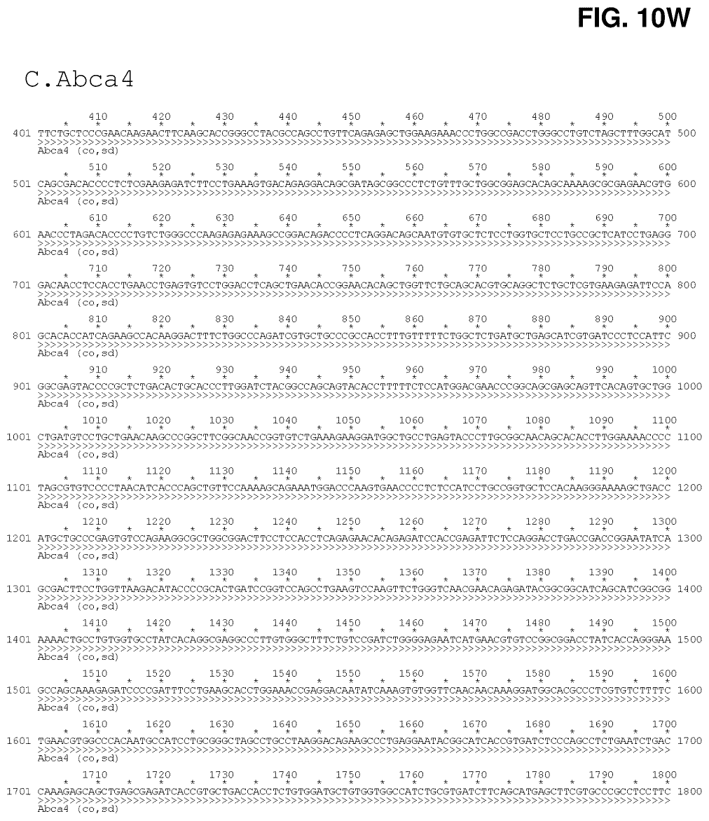

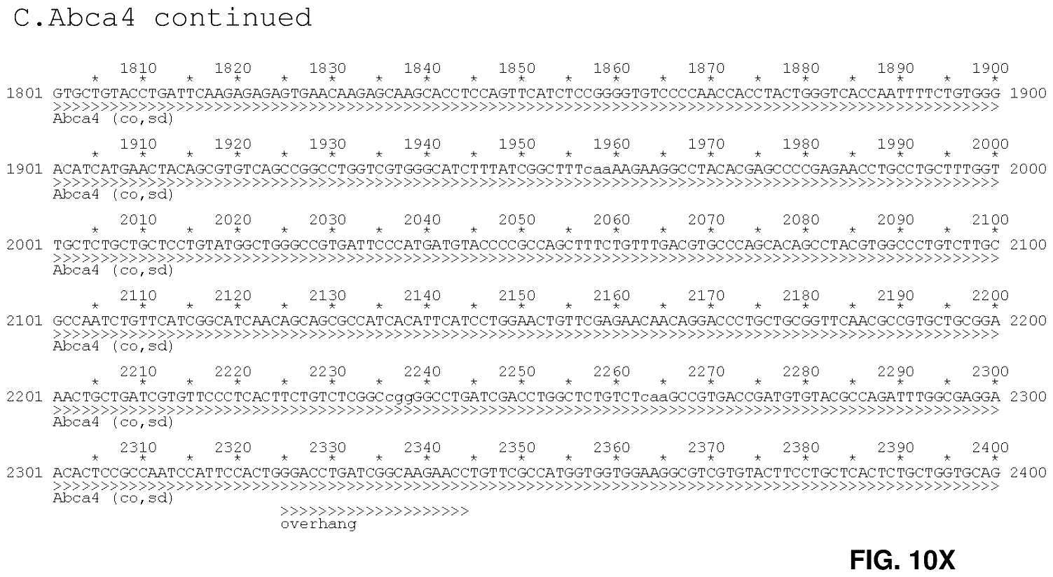

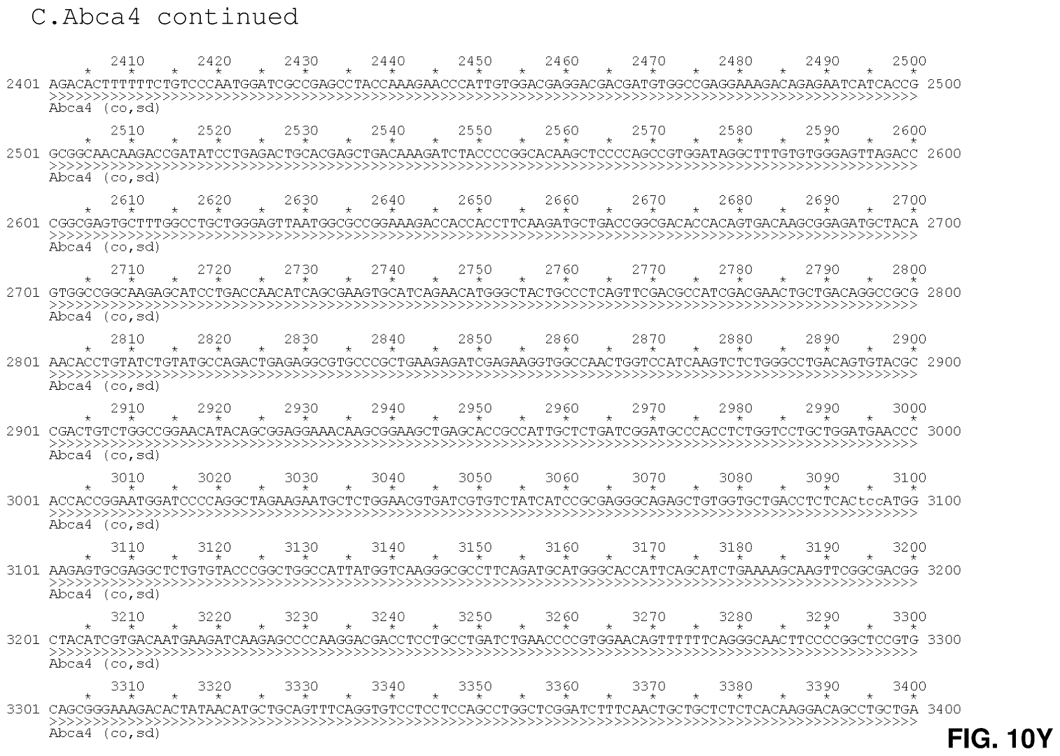

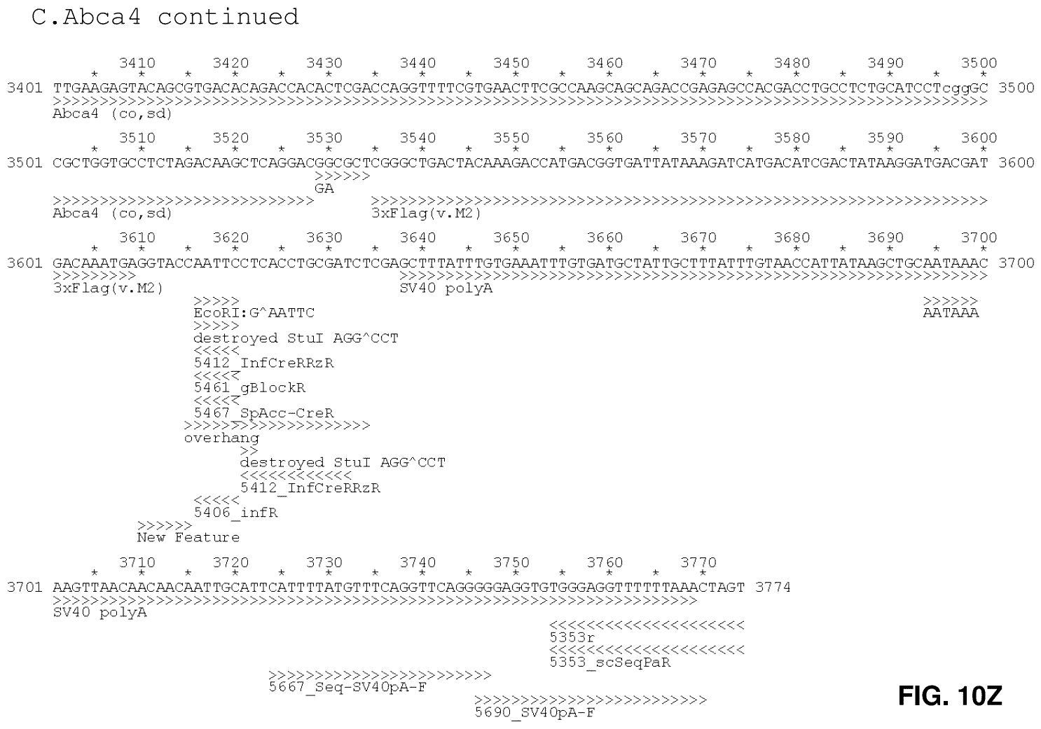

SEQ ID NO: 7 is an exemplary synthetic intron dimerization domain (FIG. 10E).

SEQ ID NO: 8 is an exemplary synthetic intron without intronic splicing enhancers (FIG. 10F).

SEQ ID NO: 9 is an exemplary synthetic intron without intronic splicing enhancers (FIG. 10G).

SEQ ID NO: 10 is an exemplary synthetic intron without intronic splicing enhancers (FIG. 10H).

SEQ ID NO: 11 is an exemplary synthetic intron without binding domain (FIG. 10I).

SEQ ID NO: 12 is an exemplary synthetic intron with dimerization domain (FIG. 10J).

SEQ ID NO: 13 is an exemplary synthetic intron with dimerization domain (FIG. 10K).

SEQ ID NO: 14 is an exemplary synthetic intron without intronic splicing enhancers (FIG. 10L).

SEQ ID NO: 15 is an exemplary synthetic intron with DISE only (FIG. 10M).

SEQ ID NO: 16 is an exemplary synthetic intron without HHrz (FIG. 10N).

SEQ ID NO: 17 is an exemplary synthetic intron without intronic splicing enhancers (FIG. 10O).

SEQ ID NO: 18 is an exemplary U12 dependent intron with binding domain (FIG. 10P).

SEQ ID NO: 19 is an exemplary U12 dependent intron with binding domain (FIG. 10Q).

SEQ ID NOS: 20 and 21 are the N- and C-terminal sequences, respectively, used to express full-length Abca4. In SEQ ID NO: 20, N-terminal Abca4 coding region nt 22 to 3702 and nt 3703 to 3975 is the synthetic intron. In SEQ ID NO: 21, nt 1 to 228 is the synthetic intron, nt 229 to 3366 C-terminal Abca4 coding region, and nt 3367 to 3611 is the untranslated poly A region.

SEQ ID NOS: 22 and 23 are the N- and C-terminal sequences, respectively, used to express a long full-length YFP, wherein each includes splice enhancers. In SEQ ID NO: 22, N-terminal YFP coding region nt 22 to 3702 and nt 3703 to 3975 is the synthetic intron. In SEQ ID NO: 23, nt 1 to 225 is the synthetic intron, nt 226 to 3747 C-terminal YFP coding region, nt 3748 to 3912 is the untranslated poly A region.

SEQ ID NOS: 24 and 25 are the N- and C-terminal sequences, respectively, used to express full-length human Factor VIII. In SEQ ID NO: 24, N-terminal FVIII coding region nt 22 to 3559 and nt 3560 to 3828 is the synthetic intron. In SEQ ID NO: 25, nt 1 to 225 is the synthetic intron, nt 226 to 3636 C-terminal FVIII coding region, and nt 3637 to 3802 is the untranslated poly A region.

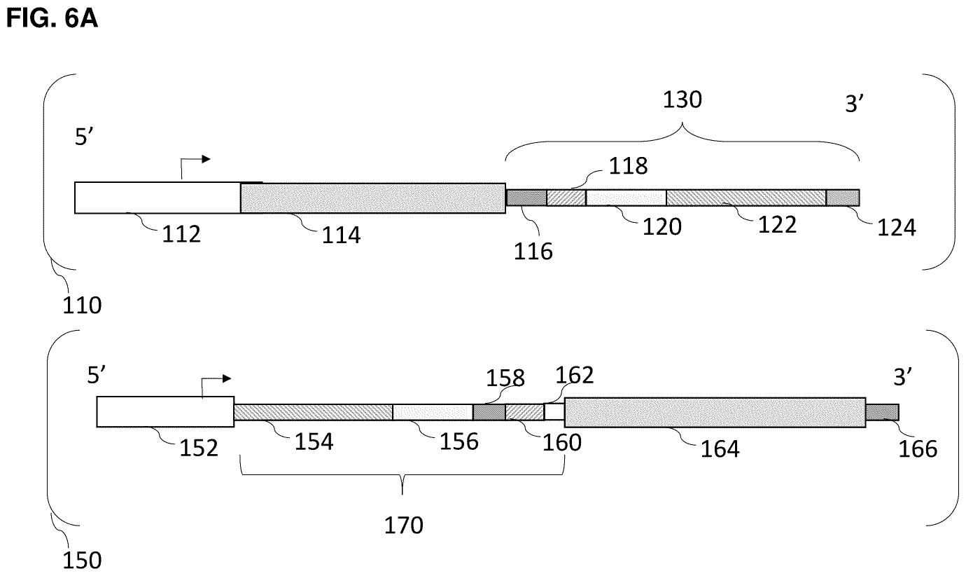

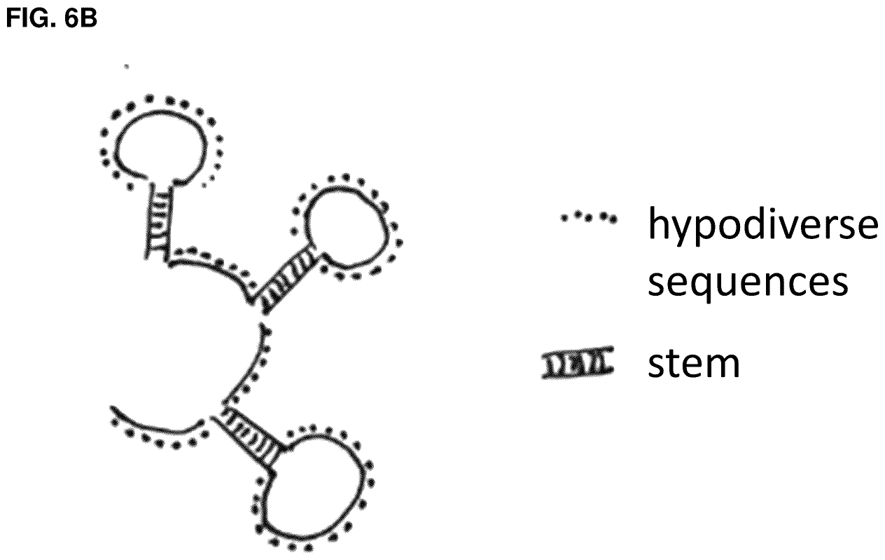

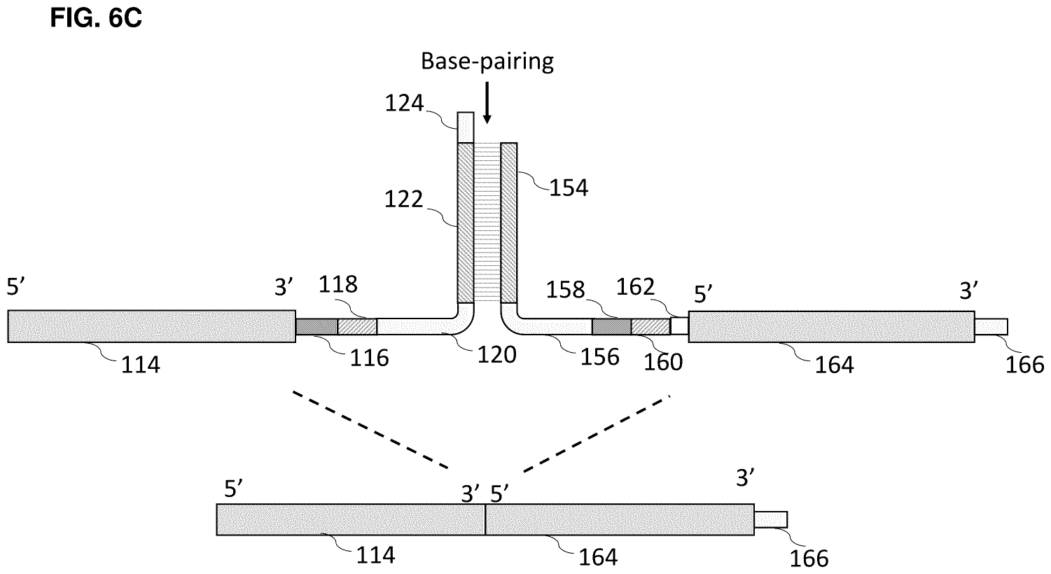

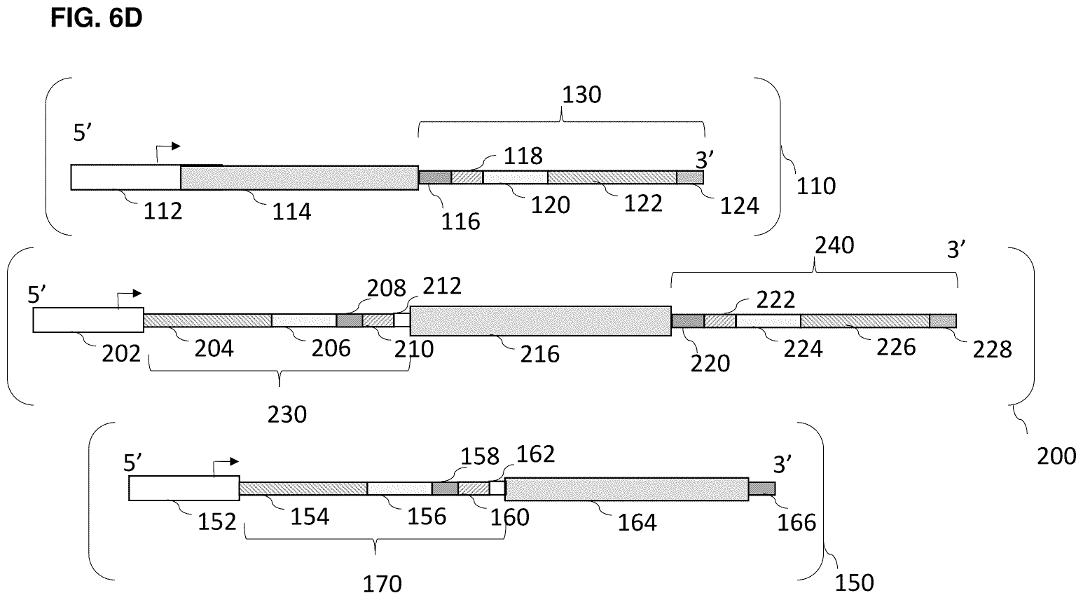

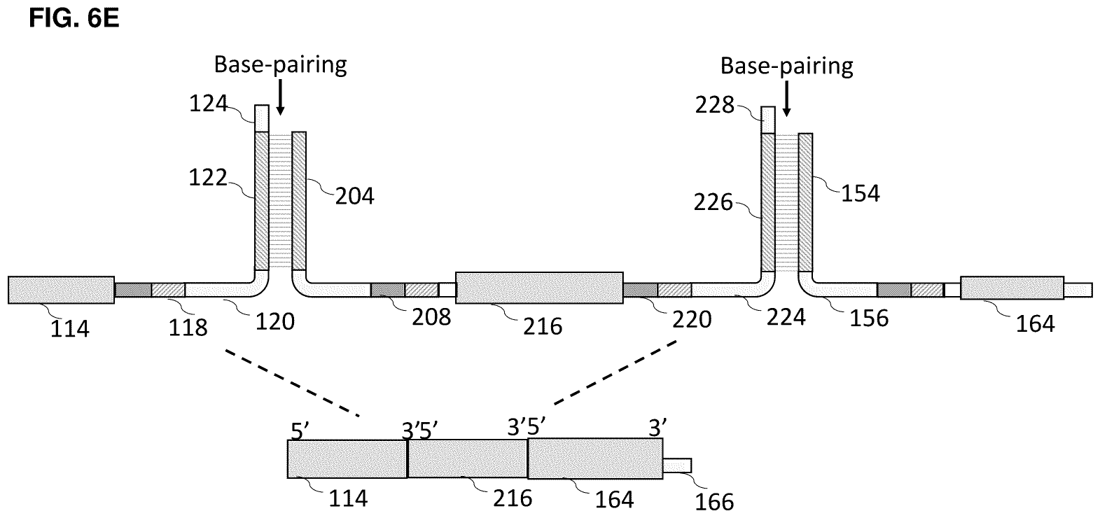

SEQ ID NOS: 26-136 are exemplary splicing enhancers that can be used with the systems provided herein (e.g., 118, 120, 156 of FIG. 6A).

SEQ ID NOS: 137 and 138 are exemplary splice donor sequences.

SEQ ID NOS: 139 and 140 are the N- and C-fragment respectively, of an HIV-1 based kissing loop dimerization domain.

SEQ ID NOS: 141 and 142 are the N- and C-fragment, respectively, of an HIV-2 based kissing loop dimerization domain.

SEQ ID NO: 143 is an exemplary cryptic splice acceptor sequence.

SEQ ID NO: 144 is an exemplary branch point consensus sequence.

SEQ ID NOS: 145 and 146 are the N- and middle sequences, respectively, used to express a long full-length YFP, along with SEQ ID NO: 2 (C-terminal fragment). In SEQ ID NO: 145, nt 1 to 543 is the CMV promoter sequence, nt 544 to 849 N-terminal YFP coding region, and nt 850 to 1305 is the synthetic intron. In SEQ ID NO: 146, nt 1 to 522 is the CMV promoter sequence, nt 523 to 901 is the synthetic intron, nt 902 to 1084 is the middle YFP coding region, and nt 1085 to 1543 is the untranslated poly A region.

SEQ ID NOS: 147 and 148 are the 5′ and 3′-synthetic sequences, respectively, used to express a long full-length Flpo. In SEQ ID NO: 147, nt 1 to 540 is the CMV promoter sequence, nt 541 to 1112 N-terminal Flpo coding region, and nt 1113 to 1571 is the synthetic intron. In SEQ ID NO: 148, nt 1 to 522 is the CMV promoter sequence, nt 523 to 904 is the synthetic intron, nt 905 to 1604 is the C-terminal Flpo coding region, nt 1605 to 1765 is the untranslated poly A region.

SEQ ID NOS: 149 and 150 are exemplary hypodiverse sequences.

SEQ ID NOS: 151 and 152 are exemplary splice donor consensus sequences.

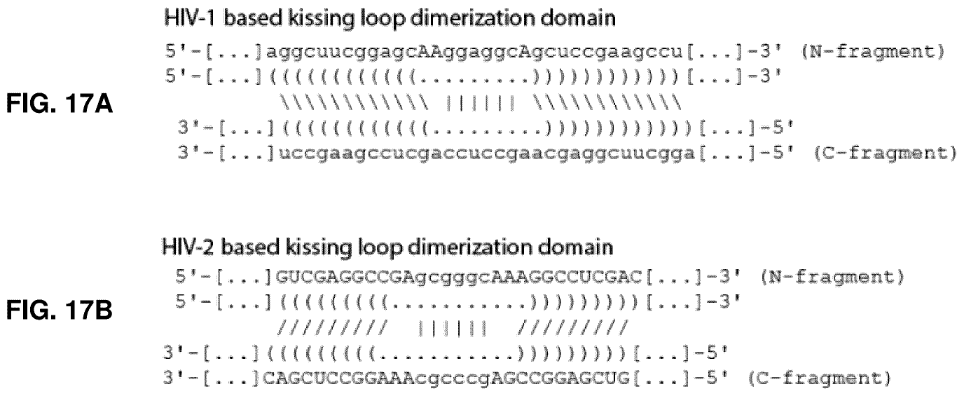

SEQ ID NO: 153 is an exemplary kissing loop based on the HIV-2 kissing loop dimerization domain (SEQ ID NOS: 141 and 142, FIG. 17B).

SEQ ID NO: 154 is an exemplary Kozak enhanced start codon.

### DETAILED DESCRIPTION

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, are herein incorporated by reference in their entireties.

In order to facilitate review of the various embodiments of the disclosure, the following explanations of specific terms are provided:
Administration: To provide or give a subject an agent, such as a therapeutic nucleic acid molecule provided herein, or other therapeutic agent, by any effective route. Exemplary routes of administration include, but are not limited to, injection (such as subcutaneous, intramuscular, intradermal, intraperitoneal, intrathecal, intratumoral, intraosseous, and intravenous), transdermal, intranasal, and inhalation routes. Administration can be systemic or local.
Aptamer: Nucleic acid molecules (such as DNA or RNA) that bind a specific target agent with high affinity and specificity. Aptamers can be used in the disclosed nucleic acid molecules as a dimerization domain, for example to allow RNA recombination only in the presence of one or more targets recognized by the aptamer. Aptamers have been obtained through a combinatorial selection process called systematic evolution of ligands by exponential enrichment (SELEX) (see for example Ellington et al., Nature 1990, 346, 818-822; Tuerk and Gold Science 1990, 249, 505-510; Liu et al., Chem. Rev. 2009, 109, 1948-1998; Shamah et al., Acc. Chem. Res. 2008, 41, 130-138; Famulok, et al., Chem. Rev. 2007, 107, 3715-3743; Manimala et al., Recent Dev. Nucleic Acids Res. 2004, 1, 207-231; Famulok et al., Acc. Chem. Res. 2000, 33, 591-599; Hesselberth, et al., Rev. Mol. Biotech. 2000, 74, 15-25; Wilson et al., Annu. Rev. Biochem. 1999, 68, 611-647; Morris et al., Proc. Natl. Acad. Sci. U.S.A. 1998, 95, 2902-2907). In such a process, DNA or RNA molecules that are capable of binding a target molecule of interest are selected from a nucleic acid library consisting of 10¹⁴-10¹⁵ different sequences through iterative steps of selection, amplification and mutation. The affinity of the aptamers towards their targets can rival that of antibodies, with dissociation constants in as low as the picomolar range (Morris et al., Proc. Natl. Acad. Sci. U.S.A. 1998, 95, 2902-2907; Green et al., Biochemistry 1996, 35, 14413-14424).

Aptamers that are specific to a wide range of targets from small organic molecules such as adenosine, to proteins such as thrombin, and even viruses and cells have been identified (Liu et al., Chem. Rev. 2009, 109, 1948-1998; Lee et al., Nucleic Acids Res. 2004, 32, D95-D100; Navani and Li, Curr. Opin. Chem. Biol. 2006, 10, 272-281; Song et al., TrAC, Trends Anal. Chem. 2008, 27, 108-117). For example, aptamers are available that recognize metal ions such as Zn (II) (Ciesiolka et al., RNA 1:538-550, 1995) and Ni(II) (Hofmann et al., RNA, 3:1289-1300, 1997); nucleotides such as adenosine triphosphate (ATP) (Huizenga and Szostak, Biochemistry, 34:656-665, 1995); and guanine (Kiga et al., Nucleic Acids Res., 26:1755-60, 1998); co-factors such as NAD (Kiga et al., Nucleic Acids Res., 26:1755-60, 1998) and flavin (Lauhon and Szostak, J. Am. Chem. Soc., 117:1246-57, 1995); antibiotics such as viomycin (Wallis et al., Chem. Biol. 4:357-366, 1997) and streptomycin (Wallace and Schroeder, RNA 4:112-123, 1998); proteins such as HIV reverse transcriptase (Chaloin et al., Nucleic Acids Res., 30:4001-8, 2002) and hepatitis C virus RNA-dependent RNA polymerase (Biroccio et al., J. Virol. 76:3688-96, 2002); toxins such as cholera whole toxin and staphylococcal enterotoxin B (Bruno and Kiel, BioTechniques, 32: pp. 178-180 and 182-183, 2002); and bacterial spores such as the anthrax (Bruno and Kiel, Biosensors & Bioelectronics, 14:457-464, 1999).
Binding: An association between two substances or molecules, such as the hybridization of one nucleic acid molecule to another (or itself), such as between two dimerization domains, or the binding of an aptamer to its target. An oligonucleotide molecule binds or stably binds to another nucleic acid molecule if there are a sufficient number of complementary base pairs between the oligonucleotide molecule and the target nucleic acid to permit detection of that binding.

C-terminal portion: A region of a protein sequence that includes a contiguous stretch of amino acids that begins at or near the C-terminal residue of the protein. A C-terminal portion of the protein can be defined by a contiguous stretch of amino acids (e.g., a number of amino acid residues).

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Thus, in some examples, a first dimerization domain and a second dimerization domain have perfect complementary to one another (e.g., 100%). In other examples, a first dimerization domain and a second dimerization domain are substantially complementary to one another (e.g., at least 80%).

Contact: Placement in direct physical association, including a solid or a liquid form. Contacting can occur in vitro or ex vivo, for example, by adding a reagent to a sample (such as one containing cells), or in vivo by administering to a subject.

Downregulated or knocked down: When used in reference to the expression of a molecule, such as a target nucleic acid or protein, refers to any process which results in a decrease in production of the target RNA or protein, but in some examples not complete elimination of the target RNA product or target RNA function. In one example, downregulation or knock down does not result in complete elimination of detectable target nucleic acid/protein expression or activity. In some examples, downregulation or knock down of a target nucleic acid includes processes that decrease translation of the target RNA and thus can decrease the presence of corresponding proteins. The disclosed system can be used to downregulate any target nucleic acid/protein of interest.

Downregulation or knock down includes any detectable decrease in the target nucleic acid/protein. In certain examples, detectable target nucleic acid/protein in a cell or cell free system decreases by at least 10%, at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of target nucleic acid/protein detected in a corresponding untreated cell or sample). In one example, a control is a relative amount of expression in a normal cell (e.g., a non-recombinant cell that does not include a nucleic acid molecule for RNA recombination provided herein).

Effective amount: The amount of an agent (such as a system providing multiple vectors, each encoding a different portion of a therapeutic protein, such as dystrophin) that is sufficient to effect beneficial or desired results. An effective amount also can refer to an amount of correctly joined RNA or therapeutic protein produced that is sufficient to effect beneficial or desired results.

An effective amount (also referred to as a therapeutically effective amount) may vary depending upon one or more of: the subject and disease condition being treated, the weight and age of the subject, the severity of the disease condition, the manner of administration and the like, which can be determined by one of ordinary skill in the art. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one embodiment, an “effective amount” of two or more synthetic nucleic acid molecules provided herein, sufficient to treat a disease, such as a genetic disease or cancer. In one embodiment, an “effective amount” of two or more synthetic nucleic acid molecules provided herein is amount sufficient to increase the survival time of a treated patient, for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase the survival time of a treated patient, for example by at least 6 months, at least 9 months, at least 1 year, at least 1.5 years, at least 2 years, at least 2.5 years, at least 3 years, at least 4 years, at least 5 years, at least 10 years, at least 12 years, at least 15 years, or at least 20 years (as compared to no administration of the two or more synthetic nucleic acid molecules provided herein). In one embodiment, an “effective amount” of two or more synthetic nucleic acid molecules provided herein is an amount sufficient to increase mobility of a treated patient (such as a DMD patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase mobility of a treated patient (such as a DMD patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase cognitive ability of a treated patient (such as a DMD patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase respiratory function of a treated patient (such as a DMD patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase blood clotting of a treated patient (such as a hemophilia patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase vision of a treated patient (such as a Usher or Stargardt patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase hearing of a treated patient (such as a Usher patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein).

In one embodiment, an “effective amount” of two or more synthetic nucleic acid molecules provided herein is an amount sufficient to reduce calf muscle size of a treated DMD patient, for example by at least 10%, at least 20%, at least 25%, at least 50%, at least 70%, at least 75%, at least 80%, at least 90%, or at least 95% (as compared to no administration of the two or more synthetic nucleic acid molecules provided herein). In one embodiment, an “effective amount” of two or more synthetic nucleic acid molecules provided herein is an amount sufficient to reduce cardiomyopathy muscle size of a treated DMD patient, for example by at least 10%, at least 20%, at least 25%, at least 50%, at least 70%, at least 75%, at least 80%, at least 90%, or at least 95% (as compared to no administration of the two or more synthetic nucleic acid molecules provided herein). In some examples, combinations of these effects are achieved.

Increase or Decrease: A statistically significant positive or negative change, respectively, in quantity from a control value (such as a value representing no therapeutic agent, such as no administration of the two or more synthetic nucleic acid molecules provided herein). An increase is a positive change, such as an increase at least 50%, at least 100%, at least 200%, at least 300%, at least 400% or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95%, or no more than 99%.

Hybridization: Hybridization of a nucleic acid occurs when two nucleic acid molecules undergo an amount of hydrogen bonding to each other. The stringency of hybridization can vary according to the environmental conditions surrounding the nucleic acids, the nature of the hybridization method, and the composition and length of the nucleic acids used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, NY, 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of nucleic acid is hybridized to its complementary strand.

Isolated: An “isolated” biological component (such as a nucleic acid molecule or a protein) has been substantially separated, produced apart from, or purified away from other biological components in the cell or tissue of an organism in which the component occurs, such as other cells (e.g., RBCs), chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

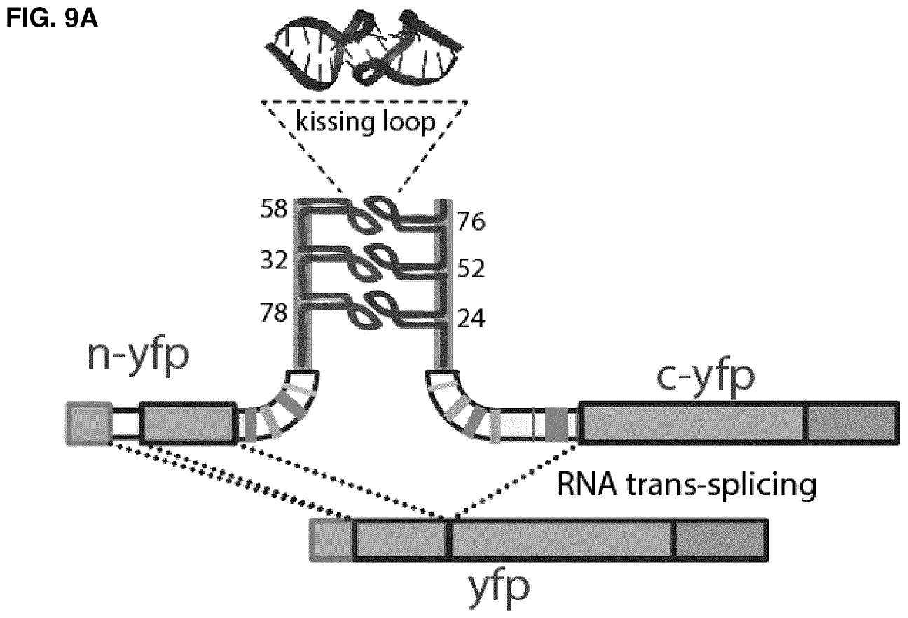

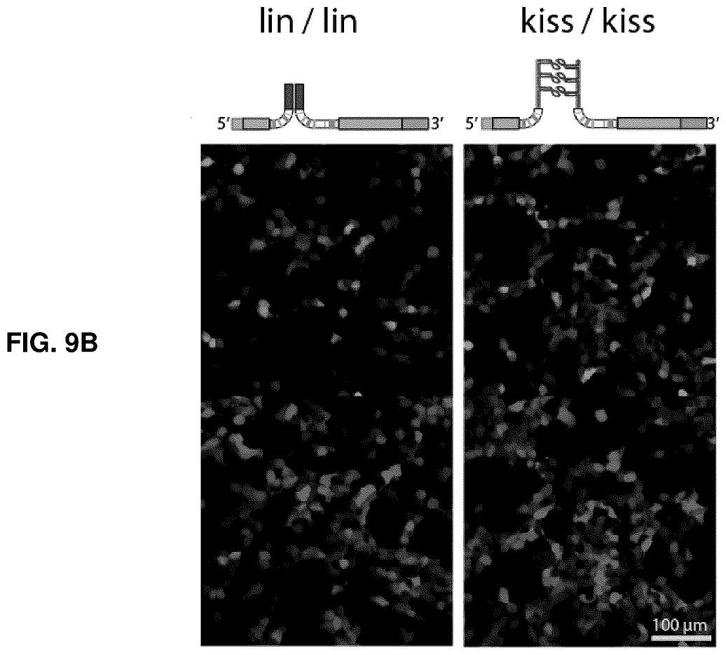

Kissing loop/kissing stem loop: An RNA structure that forms when bases between two hairpin loops form pair interactions. These intermolecular “kissing interactions” occur when the unpaired nucleotides in one hairpin loop, base pair with the unpaired nucleotides in another hairpin loop to form a stable interaction complex. See FIG. 9A for an example.

N-terminal portion: A region of a protein sequence that includes a contiguous stretch of amino acids that begins at the N-terminal residue of the protein. An N-terminal portion of the protein can be defined by a contiguous stretch of amino acids (e.g., a number of amino acid residues).

Non-naturally occurring, synthetic, or engineered: Terms used herein as interchangeably and indicate the involvement of the hand of man. The terms, when referring to nucleic acid molecules or polypeptides indicate that the nucleic acid molecule or the polypeptide is at least substantially free from at least one other component with which they are naturally associated in nature and as found in nature. In addition, the terms can indicate that the nucleic acid molecules or polypeptides have a sequence not found in nature.

Nucleic acid molecule: A deoxyribonucleotide (DNA) or ribonucleotide (RNA) polymer, which can include natural nucleotides/ribonucleotides and/or analogues of natural nucleotides/ribonucleotides that hybridize to nucleic acid molecules in a manner similar to naturally occurring nucleotides. A nucleic acid molecule can be a single stranded (ss) DNA or RNA molecule or a double stranded (ds) nucleic acid molecule.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (such as a portion of a DMD, factor 8, factor 9, or ABCA4 coding sequence). Generally, operably linked DNA sequences are contiguous and, where necessary to join two protein coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of a therapeutic agent, such as a nucleic acid molecule disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, peptide and protein: Refer to polymers of amino acids of any length. The polymer may be linear or branched, it may include modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. In one example, a protein is one associated with disease, such as a genetic disease (e.g,. see Table 1). In one example, a protein is a therapeutic protein, such as one used in the treatment of a disease, such as cancer. In one example a protein is at least 50 aa in length, at least 100 aa in length, at least 500 aa in length, at least 1000 aa in length, at least 1500 aa in length, such as at least 2000 aa, at least 2500 aa, at least 3000 aa, or at least 5000 aa.

Polypyrimidine tract: A region of pre-messenger RNA (mRNA) that promotes the assembly of the spliceosome, the protein complex specialized for carrying out RNA splicing during the process of post-transcriptional modification. This tract can be primarily pyrimidine nucleotides, such as uracil, and in some examples is 15-20 base pairs long, located about 5-40 base pairs before the 3′ end of the intron to be spliced.

Promoter/Enhancer: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. In some examples a promoter sequence+its corresponding coding sequence is larger than the capacity for an AAV. In some examples a promoter sequence of a target protein is at least 3500 nt, at least 4000 nt, at least 5000 nt, or even at least 6000 nt.

A “constitutive promoter” is a promoter that is continuously active and is not subject to regulation by external signals or molecules. In contrast, the activity of an “inducible promoter” is regulated by an external signal or molecule (for example, a transcription factor). Both constitutive and inducible promoters can be used in the methods and systems provided herein (see e.g., Bitter et al., Methods in Enzymology 153:516-544, 1987). A tissue-specific promoter can be used in the methods and systems provided herein, for example to direct expression primarily in a desired tissue or cell of interest, such as muscle, neuron, bone, skin, blood, specific organs (e.g., liver, pancreas), or particular cell types (e.g., lymphocytes). In some examples, a promoter used herein is endogenous to the target protein expressed. In some examples, a promoter used herein is exogenous to the target protein expressed.

Also included are promoter elements which are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Exemplary promoters that can be used with the methods and systems provided herein include, but are not limited to an SV40 promoter, cytomegalovirus (CMV) promoter (optionally with the CMV enhancer), a pol III promoter (e.g., U6 and H1 promoters), a pol II promoter (e.g., the retroviral Rous sarcoma virus (RSV) LTR promoter (optionally with the RSV enhancer), the dihydrofolate reductase promoter, the β-actin promoter, the phosphoglycerol kinase (PGK) promoter, and the EF1α promoter).

Recombinant: A recombinant nucleic acid molecule or protein sequence is one that has a sequence that is not naturally occurring or has a sequence that is made by an artificial combination of two otherwise separated segments of sequence (e.g., a viral vector that includes a portion of a dystrophin coding sequence, such as about a third, half, or two-thirds of a coding sequence). This artificial combination can be accomplished by, for example, chemical synthesis or the artificial manipulation of isolated segments of nucleic acids, such as by genetic engineering techniques. Similarly, a recombinant or transgenic cell is one that contains a recombinant nucleic acid molecule.

Sequence identity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of a native protein or coding sequence (such as a DMD, factor 8, factor 9, or ABCA4 sequence) are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. These sequence identity ranges are provided for guidance only; it is possible that strongly significant homologs could be obtained that fall outside of the ranges provided.

Variants of the disclosed nucleic acid sequences (such as synthetic intron sequences and coding sequences) are typically characterized by possession of at least about 80%, at least 90%, at least 95%, at least 96%, at least 97%, at least 98% or at least 99% sequence identity counted over the full length alignment with the nucleic acid sequence using the NCBI Blast 2.0, gapped blastn set to default parameters. One of skill in the art will appreciate that these sequence identity ranges are provided for guidance only; it is possible that functional sequences could be obtained that fall outside of the ranges provided.

Subject: A mammal, for example a human. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dolphin, dog, cat, horse, or cow. In some examples, the subject is a laboratory animal/organism, such as a mouse, rabbit, or rat. In some examples, the subject treated using the methods disclosed herein is a human.

In some examples, the subject has genetic disease, such as one listed in Table 1, that can be treated using the methods disclosed herein. In some examples, the subject treated using the methods disclosed herein is a human subject having a genetic disease. In some examples, the subject treated using the methods disclosed herein is a human subject having cancer

Therapeutic agent: Refers to one or more molecules or compounds that confer some beneficial effect upon administration to a subject. The disclosed synthetic nucleic acid molecules and systems provided herein are therapeutic agents. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Transduced, Transformed and Transfected: A virus or vector “transduces” a cell when it transfers nucleic acid molecules into a cell. A cell is “transformed” or “transfected” by a nucleic acid transduced into the cell when the nucleic acid becomes stably replicated by the cell, either by incorporation of the nucleic acid into the cellular genome, or by episomal replication.

These terms encompasses all techniques by which a nucleic acid molecule can be introduced into such a cell, including transfection with viral vectors, transformation with plasmid vectors, and introduction of naked DNA by electroporation, lipofection, particle gun acceleration and other methods in the art. In some example the method is a chemical method (e.g., calcium-phosphate transfection), physical method (e.g., electroporation, microinjection, particle bombardment), fusion (e.g., liposomes), receptor-mediated endocytosis (e.g., DNA-protein complexes, viral envelope/capsid-DNA complexes) and biological infection by viruses such as recombinant viruses (Wolff, J. A., ed, Gene Therapeutics, Birkhauser, Boston, USA, 1994). Methods for the introduction of nucleic acid molecules into cells are known (e.g., see U.S. Pat. No. 6,110,743). These methods can be used to transduce a cell with the disclosed nucleic acid molecules.

Transgene: An exogenous gene, for example supplied by a vector, such as AAV. In one example, a transgene encodes a portion of a target protein, such as about a third, half, or two-thirds of a target protein, for example operably linked to a promoter sequence. In one example, a transgene includes a portion of a dystrophin coding sequence, such as about a third, half, or two-thirds of a dystrophin coding sequence (or other therapeutic coding sequence, such as one encoding a protein listed in Table 1), for example operably linked to a promoter sequence.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, blood and other clinical tests, and the like. In some examples, treatment with the disclosed methods results in a decrease in the number or severity of symptoms associated with a genetic disease, such as increasing the survival time of a treated patient with the genetic disease.

In some examples, treatment with the disclosed methods results in a decrease in the number or severity of symptoms associated with DMD or other genetic disease, such as increasing survival, increasing the mobility (e.g., walking, climbing), improving cognitive ability, reducing calf muscle size, reduce cardiomyopathy, improving vision, improving hearing, improving blood clotting, or improve respiratory function. In some examples, combinations of these effects are achieved.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated with the disclosed methods and systems include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma.

The methods and systems can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas).

Upregulated: When used in reference to the expression of a molecule, such as a target nucleic acid/protein, refers to any process which results in an increase in production of the target nucleic acid/protein. In some examples, upregulation or activation of a target RNA includes processes that increase translation of the target RNA and thus can increase the presence of corresponding proteins.

Upregulation includes any detectable increase in target nucleic acid/protein. In certain examples, detectable target nucleic acid/protein expression in a cell or cell free system increases by at least 20%, at least 30%, at least 40%, at least 50%, at least 60%, at least 70%, at least 75%, at least 80%, at least 90%, at least 95%, at least 100%, at least 200%, at least 400%, or at least 500% as compared to a control (such an amount of target nucleic acid/protein detected in a corresponding sample not treated with a nucleic acid molecule provided herein). In one example, a control is a relative amount of expression in a normal cell (e.g., a non-recombinant cell that does not include a system provided herein).

Under conditions sufficient for: A phrase that is used to describe any environment that permits a desired activity. In one example the desired activity is increased expression or activity of a protein needed to treat a disease. In one example the desired activity is treatment of or slowing the progression of a genetic disease such as DMD (or other genetic disease listed in Table 1) in vivo, for example using the disclosed methods and systems.

Vector: A nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides.

A vector can include nucleic acid sequences that permit it to replicate in a host cell, such as an origin of replication. A vector can also include one or more selectable marker genes and other genetic elements. An integrating vector is capable of integrating itself into a host nucleic acid. An expression vector is a vector that contains the necessary regulatory sequences to allow transcription and translation of inserted gene or genes.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector.

In some embodiments, the vector is an AAV, such as AAV serotypes AAV9 or AAVrh.10. In some embodiments, the vector is one that can penetrate the blood-brain barrier, for example following intravenous administration. The adeno-associated virus serotype rh.10 (AAV.rh10) vector partially penetrates the blood-brain barrier, providing high levels and spread of transgene expression.

### II. Overview of Several Embodiments

One approach to curing patients who suffer from genetic diseases is gene replacement therapy (generally referred to as gene therapy). In such an approach, the defective gene is replaced by an intact version of it, delivered through e.g., a viral vector, which achieves sustained expression from months to years. Although adeno associated viruses (AAVs) have been used for clinical gene replacement therapy, they have a limited packaging capacity (e.g., about less than 5 kb). Thus, strategies to overcome this packaging limitation are needed to achieve gene replacement of genes that exceed the about 5 kb size limit. For example some promoters alone, coding sequences alone, or the combined promoter+coding sequence, exceed the about 5 kb size limit of an AAV. Thus, such proteins encoded by such promoters and coding sequences can be expressed using the disclosed systems.

Prior methods to overcome the cargo limitations of AAV do not appear to achieve the efficiency required to produce adequate levels of target protein in sufficient numbers of cells to treat disease. For example as dystrophin is about 11 kb, it needs to be delivered in a minimum of three fragments to be compatible with AAV packaging limitations.

Splicing mediated recombination of two RNA molecules using naturally occurring intron sequences for one or both of the RNA fragments is inefficient. First, these natural intron sequences are sequences from naturally occurring introns and are comprised of a mix of all four RNA nucleotides. Such sequences tend to fold up into structures that can obstruct trans-interaction by forming strong intramolecular base pairs rather than being available for intermolecular interactions. Second, these naturally occurring intron sequences have not evolved to strongly attract the spliceosome components, since exon rather than introns drive the exon definition in higher eukaryotes. These two limitations of previous strategies are addressed herein by designing synthetic intronic sequences that are not found in nature. These synthetic sequences contain elements that strongly attract and stimulate spliceosome recruitment on the one hand while minimizing the secondary structure (and in some examples other structure, such as tertiary structure) that obstructs bringing the two RNA fragments together.

The inventors developed a novel RNA based element that can be used to efficiently reconstitute the coding sequence of large genes from multiple serial fragments. The disclosed methods and systems differ from prior methods. The disclosed highly efficient synthetic introns utilize an optimal arrangement of RNA elements that efficiently drive the RNA splicing reaction between non-covalently linked RNAs. The method/system is a significant advancement over previous attempts to harness trans-splicing because it generates high levels of functional protein that more closely approximate the therapeutic levels of a protein to treat genetic diseases. The innovation is based on selecting non-natural RNA domains that inherently are incapable of forming strong cis-binding interactions that interfere with trans-interactions with a second RNA having a complementary strand (also having inherently low cis-binding capacity). These optimized dimerization domains are non-natural sequences (e.g., sequences are not found in human cells) used in combination with optimized motifs that facilitate RNA splicing (including splice donor, splice acceptor, splice enhancer, and splice branch point sequences). By optimizing the trans-dimerization of the RNA strands in the context of the appropriate RNA motifs that mediate efficient splicing, it is demonstrated herein for the first time that two or three different RNAs can be precisely and efficiently covalently linked in the same cell producing high levels of functional proteins in vivo and in vitro. Unlike the “hybrid” approach that provides an inefficient combination at the DNA level via DNA recombination that is ultimately followed by RNA splicing in cis to excise the DNA recombination site from the mature transcript, the disclosed method/system promotes a more efficient reaction in which two protein coding RNA fragments are joined together on the pre-mRNA level with less risk of producing recombination products that encode non-functional and/or deleterious products.

The data demonstrate that by using efficient synthetic RNA-dimerization and recombination domains (sRdR domains, also referred to as RNA end-joining (REJ) domains), a gene of interest can efficiently reconstitute from two or three separate gene fragments expressed in the same cell. These results show the ability of the disclosed methods and systems to reconstitute large genes like dystrophin or the blood clotting Factor VIII, or the ATP binding cassette subfamily A member 4 (Abca4) using AAVs, in order to treat Duchenne Muscular Dystrophy and Hemophilia A, or Stargardt's Disease respectively. Based on these observations, other genetic diseases can be similarly treated, such as ones benefiting from expression of a large protein (e.g., see disorders listed in Table 1). Other applications include research and biotechnology applications.

To address some of the limitations with existing strategies for reconstitution of fragmented genes from multiple AAVs, provided herein is a system that serially aligns and recombines two or more individual synthetic RNA molecules in the target cell. Each individual synthetic RNA molecule includes a synthetic intron sequence, containing a dimerization domain and elements needed for RNA splicing, which upon binding of dimerization domains to one another in the correct order, mediates efficient RNA recombination of individual fragments. In one example, reconstitution of a coding sequence from two fragments is achieved by appending a first synthetic intron (A) to the 3′ end of the N-terminal coding fragment and a complimentary second synthetic domain (A′) to the 5′ end of the C-terminal coding fragment. The two RNAs are recombined by a cell's intrinsic RNA splicing machinery (i.e., the spliceosome machinery). The synthetic intron domains contain two functional elements: (1) a dimerization domain to mediate base pairing between the two halves that are to be recombined and (2) a domain optimized to efficiently recruit the splicing machinery to mediate efficient reconstitution of the two RNA molecules. In some examples, a synthetic intron includes a sequence having at least 50% at least 60%, at least 70%, at least 75%, 80%, at least 85%, at least 90%, at least 95%, at least 98%, at least 99%, or 100% sequence identity to any synthetic intron provided in SEQ ID NOS: 1, 2, 3, 4, 5, 6, 7, 8, 9, 10, 11, 12, 13, 14, 15, 16, 17, 18, 19, 20, 21, 22, 23, 24, 25, 145, 146, 17, and 148 (e.g., see FIGS. 10A-10Z). One skilled in the art will appreciate that any of the molecules provided in SEQ ID NOS: 1, 2, 20, 21, 22, 23, 24, 25, 145, 146, 17, and 148 can be modified to replace the protein coding portions (e.g., 114 and 164 of FIG. 6A) with another protein coding sequence of interest (e.g., YFP coding sequence of SEQ ID NO: 1, 2, 22 or 23 can be replaced with a therapeutic protein coding sequence). Thus, also provided herein are synthetic intron molecules having at least 80%, at least 85%, at least 90%, at least 95%, at least 98%, at least 99%, or 100% sequence identity to any synthetic intron portion provided in SEQ ID NO: 1, 2, 20, 21, 22, 23, 24, 25, 145, 146, 17, and 148 (e.g., nt 3703-3975 of SEQ ID NO: 22 and nt 1-225 of SEQ ID NO: 23).

Exemplary dimerization domains were bioinformatically selected to minimize/optimize their internal secondary/tertiary structure. The dimerization domains tested contained long stretches of low diversity nucleotide sequences to avoid intramolecular annealing. By avoiding intramolecular annealing, these dimerization domains are present in an open configuration and therefore are available for pairing with the corresponding complementary dimerization domain sequence. The synthetic intron domains contain intronic splice enhancing elements which lead to efficient recruitment of the splicing machinery.

The disclosed synthetic RNA molecules are designed to have at least an open and available single-stranded region that is available to bind to the complementary dimerization domain to allow efficient splicing and recombination of the RNAs. In some examples, this is achieved by utilizing only purines or only pyrimidines for the binding domains. Due to the inability of purines to pair with themselves (and pyrimidines likewise) these stretches of RNA have an open predicted structure.

RNA molecules are present as a single strand in the cells. Being single stranded they are inherently prone to hybridize to themselves and thereby form strong secondary and tertiary structures. The most stable base pairs will be G with C, A with U, and the G with U wobble pair. Thermodynamically, the pairing of two bases is favored over an open configuration. To design efficient synthetic nucleic acid molecules, the two dimerization domains having reverse complementary to one another are present in an open configuration such that the dimerization domains are available for inter-molecular base pairing. To avoid intra-molecular base pairing in between other parts of the synthetic nucleic acid molecules, a long stretch of non-diverse sequences containing incompatible bases can be included. For example, a long stretch of pyrimidines (i.e., C and T) or purines (i.e., A and G) can be present in the synthetic nucleic acid molecules. Pyrimidines cannot form canonical base pairs with other pyrimidines, purines cannot form canonical base pairs with other purines. Such a stretch of purines or pyrimidines can range from a couple bases to a couple hundreds of bases. Since these stretches cannot intra-molecularly bind, they are available for inter-molecular base pairing with a complementary fragment. For example, the synthetic nucleic acid molecules A and A′ may be configured with A containing a pyrimidine stretch (e.g., 5′-CCUU( . . . )CCUU-3′) and A′ containing the complementary purine sequence (e.g., 5′-AAGG( . . . )AAGG-3′).

The disclosed synthetic RNA molecules are designed to minimize any off-target binding to incorrect sites in the genome. Off target binding can be reduced by altering the sequence of the nucleic acid molecule.

The same design principle, that is the use of hypodiverse stretches of RNA bases to achieve open synthetic nucleic acid configurations, can be extended to using stretches of single bases e.g. using a series of Gs that would base pair with a series of Cs and a series of As that would base pair with a series of Us, in the dimerization domains.

To increase recombination of two or more synthetic nucleic acid molecules, the following methods can be used. RNA splicing depends on the recruitment of spliceosome components to the 5′ end of the intron (the splice donor site) and the 3′ end of the intron (the splice acceptor site, with its associated branch point sequence and the polypyrimidine tract). Different ribonucleoproteins are recruited to the intron through base pairing of protein associated small nuclear RNA (snRNA) with intronic sequences. By placing perfect match consensus sequences into the RNA dimerization and recombination domains, the recruitment of spliceosome components can be facilitated which in turn enhances the efficiency of spliceosome mediated recombination. Previously characterized intronic splice enhancer sequences can recruit additional splicing promoting factors that are referred to as intronic splice enhancers.

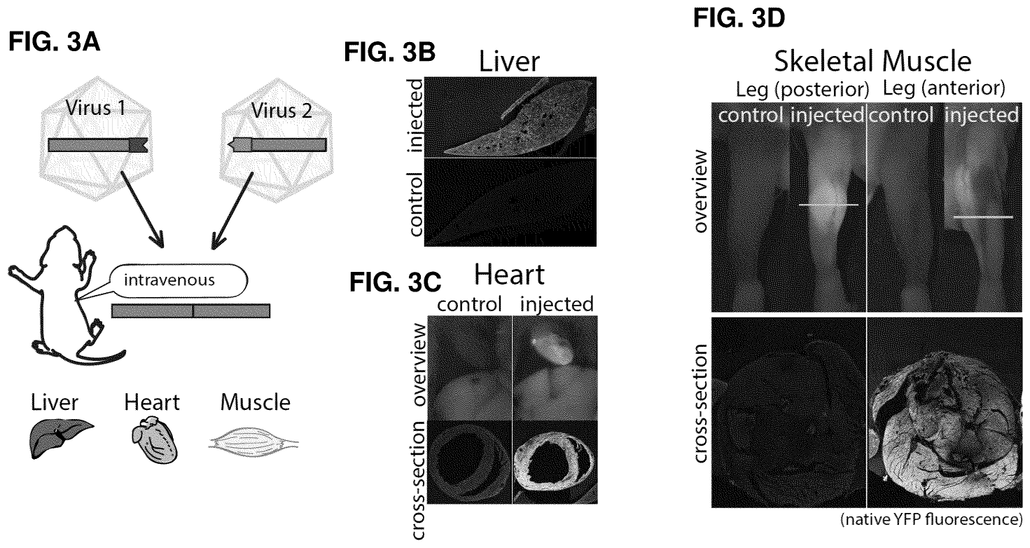

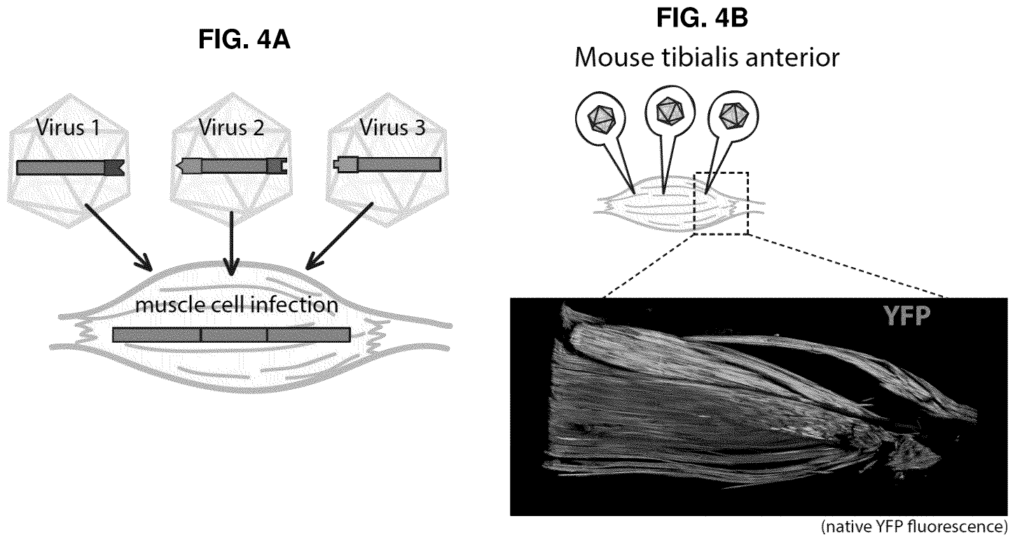

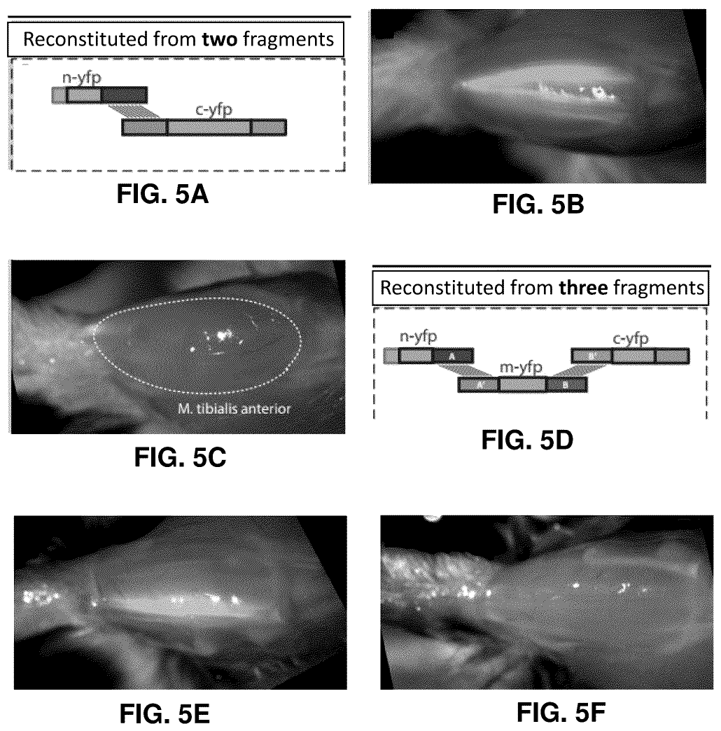

In some examples, instead of using naturally occurring RNA sequences for the RNA splicing sequences, consensus sequences are used. For example, consensus sequences can be used for any of the sequences that are involved in splicing, including splice donor, splice acceptor, splice enhancer and splice branch point sequences. With these synthetic nucleic acid molecules, two (or more) RNA molecules can be serially joined together in a cell ex vivo, in vitro, or in vivo. Outside of the synthetic intronic domains, synthetic nucleic acid molecules can include any promoter and coding sequence. For example, two synthetic nucleic acid molecules could carry two halves of a single gene. This was tested in vitro and in vivo by reconstituting two halves of a yellow fluorescent protein (YFP), and was shown to be efficient (see FIGS. 3A-3D).

The modular nature of the synthetic nucleic acid molecules allowed for the testing the efficiency of achieving serial recombination (i.e., >2) of multiple RNA fragments using a combinatorial set of optimized complimentary dimerization domains (FIGS. 4A-4D). A three-way split yellow fluorescent protein was efficiently reconstituted and expressed at high levels in >80% of transfected cells.

These results demonstrate that a single RNA molecule can be reconstituted from at least three different synthetic nucleic acid molecules, such as when expression of a disease causing gene (or therapeutic protein) that has a promoter and/or a coding sequence that is too long to fit into a single gene therapy vector such as AAV.

The disclosed system allows for the efficient RNA recombination between individual fragments. In some examples, reconstitution (i.e., splicing or recombination) efficiency achieved using the compositions, systems or methods of the disclosure is determined using any suitable method known to one of skill in the art. In some examples, reconstitution efficiency is represented by a measure of correctly joined RNA relative to a control RNA, or a measure of full-length protein or protein activity relative to that of a control protein. In some examples the control RNA is the unjoined RNA, wherein reconstitution efficiency is represented by a measure of joined RNA relative to unjoined RNA. This measurement can be made by detecting and comparing junction RNA and the unjoined 3′ RNA species 3′ (e.g., junction RNA: 3′ RNA). In some examples wherein more than two RNAs are joined, joining at either or all junctions are evaluated. In some examples, reconstitution efficiency is represented by a measure of full-length or active protein relative to a protein fragment or inactive protein.

In some examples, the reconstitution, recombination or splicing efficiency (a measure of the correct joining of the two or more different coding sequences present on different RNA molecules, and/or the production of the desired full-length protein) is about 10% to about 100%. In some examples, the reconstitution efficiency is about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 100%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 100%, about 20% to about 25%, about 20% to about 30%, about 20% to about 40%, about 20% to about 50%, about 20% to about 60%, about 20% to about 70%, about 20% to about 80%, about 20% to about 90%, about 20% to about 100%, about 25% to about 30%, about 25% to about 40%, about 25% to about 50%, about 25% to about 60%, about 25% to about 70%, about 25% to about 80%, about 25% to about 90%, about 25% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some examples, the reconstitution efficiency is about 10%, about 15%, about 20%, about 25%, about 30%, about 40%, about 50%, about 60%, about 70%, about 80%, about 90%, or about 100%. In some examples, the reconstitution efficiency is at least about 10%, about 15%, about 20%, about 25%, about 30%, about 40%, about 50%, about 60%, about 70%, about 80%, or about 90%. In some examples, the reconstitution efficiency is at most about 15%, about 20%, about 25%, about 30%, about 40%, about 50%, about 60%, about 70%, about 80%, about 90%, or about 100%.

In some examples, the compositions, systems or methods of the disclosure are evaluated by determining an RNA or protein production level using any suitable method known to one of skill in the art. In some examples, the RNA production level is represented by a measure of correctly joined RNA relative to a control RNA, or a measure of full-length protein relative to a control. In some examples the control RNA is a corresponding mutant RNA or an endogenous RNA. For example, the ratio of the amount of joined RNA to the amount of mutant or endogenous RNA produced in the transfected cell is compared with same ratio in nontransfected cells, to determine the production level of the correctly joined RNA. In some examples, the ratio of the amount of the correctly joined RNA, full-length protein, or the protein activity, to the amount of the control RNA, or the amount or activity of the control protein, are compared.

In some examples, the RNA production level achieved is 5% to 100%. In some examples, the RNA production level achieved is about 5% to about 100%. In some examples, the RNA production level achieved is about 5% to about 10%, about 5% to about 20%, about 5% to about 25%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 100%, about 20% to about 25%, about 20% to about 30%, about 20% to about 40%, about 20% to about 50%, about 20% to about 60%, about 20% to about 70%, about 20% to about 80%, about 20% to about 90%, about 20% to about 100%, about 25% to about 30%, about 25% to about 40%, about 25% to about 50%, about 25% to about 60%, about 25% to about 70%, about 25% to about 80%, about 25% to about 90%, about 25% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some examples, the RNA production level achieved is about 5%, about 10%, about 20%, about 25%, about 30%, about 40%, about 50%, about 60%, about 70%, about 80%, about 90%, or about 100%. In some examples, the RNA production level achieved is at least about 5%, about 10%, about 20%, about 25%, about 30%, about 40%, about 50%, about 60%, about 70%, about 80%, or about 90%. In some examples, the RNA production level achieved is at most about 10%, about 20%, about 25%, about 30%, about 40%, about 50%, about 60%, about 70%, about 80%, about 90%, or about 100%.

In some examples, the protein production level is represented by a measure of the amount of full-length protein or protein activity relative to that of a control protein. In some examples the control protein is a corresponding mutant protein or an endogenous protein. For example, the ratio of the amount of full-length protein or protein activity to the amount of mutant or endogenous protein produced in the transfected cell is compared with same ratio in nontransfected cells. In some examples, the control protein is the full-length protein produced in, e.g., a cell that is engineered to express a control full-length protein (wherein the cell is not transfected with the inventive constructs) or a non-transfected cell from a normal subject that expresses a control full-length protein, and the protein production level is determined by measuring the amount or activity of the protein in the transfected cell and comparing it to that of the control protein. In some examples, the control protein is a mutant form of the protein, produced in a cell that is transfected or nontransfected with the construct, and the amount of full-length protein or protein activity is compared with that of the control protein to determine the protein production level. In some examples, the amount of full-length protein or protein activity is compared with that of an endogenous, or housekeeping, protein to determine the protein production level.

In some examples, the protein production level achieved is about 1% to about 100%. In some examples, the protein production level achieved is about 10% to about 100%. In some examples, the protein production level achieved is about 10% to about 20%, about 10% to about 30%, about 10% to about 40%, about 10% to about 50%, about 10% to about 60%, about 10% to about 70%, about 10% to about 75%, about 10% to about 80%, about 10% to about 85%, about 10% to about 90%, about 10% to about 100%, about 20% to about 30%, about 20% to about 40%, about 20% to about 50%, about 20% to about 60%, about 20% to about 70%, about 20% to about 75%, about 20% to about 80%, about 20% to about 85%, about 20% to about 90%, about 20% to about 100%, about 30% to about 40%, about 30% to about 50%, about 30% to about 60%, about 30% to about 70%, about 30% to about 75%, about 30% to about 80%, about 30% to about 85%, about 30% to about 90%, about 30% to about 100%, about 40% to about 50%, about 40% to about 60%, about 40% to about 70%, about 40% to about 75%, about 40% to about 80%, about 40% to about 85%, about 40% to about 90%, about 40% to about 100%, about 50% to about 60%, about 50% to about 70%, about 50% to about 75%, about 50% to about 80%, about 50% to about 85%, about 50% to about 90%, about 50% to about 100%, about 60% to about 70%, about 60% to about 75%, about 60% to about 80%, about 60% to about 85%, about 60% to about 90%, about 60% to about 100%, about 70% to about 75%, about 70% to about 80%, about 70% to about 85%, about 70% to about 90%, about 70% to about 100%, about 75% to about 80%, about 75% to about 85%, about 75% to about 90%, about 75% to about 100%, about 80% to about 85%, about 80% to about 90%, about 80% to about 100%, about 85% to about 90%, about 85% to about 100%, or about 90% to about 100%. In some examples, the protein production level achieved is about 10%, about 20%, about 30%, about 40%, about 50%, about 60%, about 70%, about 75%, about 80%, about 85%, about 90%, or about 100%. In some examples, the protein production level achieved is at least about 10%, about 20%, about 30%, about 40%, about 50%, about 60%, about 70%, about 75%, about 80%, about 85%, or about 90%. In some examples, the protein production level achieved is at most about 20%, about 30%, about 40%, about 50%, about 60%, about 70%, about 75%, about 80%, about 85%, about 90%, or about 100%.

In some examples, the protein activity level achieved is about 50% to about 100%. In some examples, the protein activity level achieved is about 50% to about 100%. In some examples, the protein activity level achieved is about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some examples, the protein activity level achieved is about 50%, about 55%, about 60%, about 65%, about 70%, about 75%, about 80%, about 85%, about 90%, about 95%, or about 100%. In some examples, the protein activity level achieved is at least about 50%, about 55%, about 60%, about 65%, about 70%, about 75%, about 80%, about 85%, about 90%, or about 95%. In some examples, the protein activity level achieved is at most about 55%, about 60%, about 65%, about 70%, about 75%, about 80%, about 85%, about 90%, about 95%, or about 100%.

In some examples, the amount of correctly joined RNA or full-length protein produced in a cell is sufficient to ameliorate or cure a condition or disease in a subject, as understood by one of skill in the art for the particular condition or disease. In some examples, the amount of correctly joined RNA or full-length protein produced in a cell is an effective amount. In some examples, this amount is equivalent to about 50% to 100% the amount of the RNA or protein produced in a normal cell. In some examples, this amount is equivalent to about 40% to about 100% the amount of the RNA or protein produced in a normal cell. In some examples, this amount is equivalent to about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 100%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 100%, about 55% to about 60%, about 55% to about 65%, about 55% to about 70%, about 55% to about 75%, about 55% to about 80%, about 55% to about 85%, about 55% to about 90%, about 55% to about 100%, about 60% to about 65%, about 60% to about 70%, about 60% to about 75%, about 60% to about 80%, about 60% to about 85%, about 60% to about 90%, about 60% to about 100%, about 65% to about 70%, about 65% to about 75%, about 65% to about 80%, about 65% to about 85%, about 65% to about 90%, about 65% to about 100%, about 70% to about 75%, about 70% to about 80%, about 70% to about 85%, about 70% to about 90%, about 70% to about 100%, about 75% to about 80%, about 75% to about 85%, about 75% to about 90%, about 75% to about 100%, about 80% to about 85%, about 80% to about 90%, about 80% to about 100%, about 85% to about 90%, about 85% to about 100%, or about 90% to about 100% the amount of the RNA or protein produced in a normal cell. In some examples, this amount is equivalent to about 40%, about 45%, about 50%, about 55%, about 60%, about 65%, about 70%, about 75%, about 80%, about 85%, about 90%, or about 100% the amount of the RNA or protein produced in a normal cell. In some examples, this amount is equivalent to about at least about 40%, about 45%, about 50%, about 55%, about 60%, about 65%, about 70%, about 75%, about 80%, about 85%, or about 90% the amount of the RNA or protein produced in a normal cell. In some examples, this amount is equivalent to about at most about 45%, about 50%, about 55%, about 60%, about 65%, about 70%, about 75%, about 80%, about 85%, about 90%, or about 100% the amount of the RNA or protein produced in a normal cell.

The measurements of RNA or protein used to determine recombination efficiency or production level can be made by any suitable method known to those of skill in the art. In some examples, recombination efficiency or production level is determined by measuring an amount of functional protein expressed, for example by Western blotting. In some examples, recombination efficiency or production level is determined by measuring the RNA transcript, for example using two probe based quantitative real-time PCR. For example, the first assay spans a sequence fully contained in the 3′ exonic coding sequence (labelled 3′ probe). The second assay spans the junction between the 5′ and the 3′ exonic coding sequence (labelled junction probe). Reconstitution efficiency can be calculated as the ratio of (junction probe count)/(3′ probe count). “Reconstitution efficiency,” “recombination efficiency,” and “splicing efficiency” are used interchangeably herein.

In some examples, a dimerization domain is about 20 to about 1000 nt, or about 50 to about 160 nt, or about 50 to about 500 nt, or about 50 to 1000 nt, wherein reconstitution efficiency results in production of an effective amount of correctly joined RNA or full-length protein. In some examples, a dimerization domain is about 50 to about 160 nt, wherein reconstitution efficiency results in production of an effective amount of correctly joined RNA or full-length protein.

Achieving efficient recombination between multiple RNA molecules allows for packaging and delivery of transgenes into AAVs, which exceed the packaging limit of a single AAV. AAV packaging limits represent a major hurdle for gene therapy approaches for diseases caused by the absence/defect of large genes. One application of this system is expression of large disease-causing genes using viral vectors with restricted packaging capacity. Disease and genes include but are not limited to (Disease (gene, OMIM gene identifier)): 1) Duchenne muscular dystrophy and Becker muscular dystrophy (dystrophin, OMIM:300377); 2) Dysferlinopathies (Dysferlin, OMIM:603009); 3) Cystic fibrosis (CFTR, OMIM:602421); 4) Usher's Syndrome 1B (Myosin VIIA, OMIM:276903); 5) Stargardt disease 1 (ABCA4, OMIM:601691); 6) Hemophilia A (Coagulation Factor VIII, OMIM:300841); 7) Von Willebrand disease (von Willebrand Factor, OMIM:613160); 8) Marfan Syndrome (Fibrillin 1, OMIM:134797); and 9) Von Recklinghausen disease (neurofibromatosis-1, OMIM:162200). Others are provided in Table 1. Delivery of a transgene can be achieved by splitting it into multiple fragments using the approach provided herein.

Additional applications of the disclosed methods and systems include intersectional gene delivery for targeted gene expression. One can make use of differential infection/expression patterns of two viruses encoding a fragmented gene. The reconstituted protein will get expressed in an overlapping population of cells that represents the intersection of what either virus would express in on its own. Examples for such an application may include: (1) delivery of two halves (or three thirds, or other portions) of a protein using retrogradely transported viral vectors from two (or more) projection targets to label bifurcating dual projection neurons, (2) delivery of one fragment under the control of a promoter that is active in population A and the second fragment from a promoter active in population B to specifically tag/manipulate the AUB population, (3) delivery of the first half of a protein with a viral vector that has a tropism for population A and the second half with a viral vector that has a tropism for population B to specifically tag/manipulate the AUB population. Or, combinations of these approaches.

In one example the dimerization domains are aptamer sequences, for example to facilitate dimerization in the presence of a (a) small molecular trigger recognized by the aptamers, a (b) protein that is present in the cell binding to the two halves and therefore stimulating dimerization, or (c) an antisense oligonucleotide sequence with homology to the two halves (RNA triggered dimerization). In such an example, an antisense oligonucleotide having a complementariy sequence to both halves bridges the two molecules together, thus facilitating spliceosome mediated recombination of the two molecules.

These molecule, protein, or RNA mediated interactions allow for controllable/fine tuned gene expression levels: Through titrating in molecules that interact with the binding domains (e.g., antisense oligonucleotides), dimerization efficiency between the two halves can be modulated to regulate expression levels independent of promoter activity. Such an installment can be used if a narrow range of protein expression levels are needed.

### III. Systems

Provided herein is a system that can be used to recombine two or more RNA molecules, such as at least two, at least three, at least four, or at least five different RNA molecules (such as 2, 3, 4, 5, 6, 7, 8, 9 or 10 different RNA molecules) using synthetic introns containing dimerization sequences. Unlike fragmentation and reconstitution of two fragments at the protein level, the disclosed approach does not require extensive protein engineering to find a suitable split point. Reconstitution on an RNA level allows for seamless joining of two fragments of a protein. The disclosed methods and systems allow for large genes (and corresponding proteins), such as those greater than about 4.5 kb, at least 5 kb, at least 5.5 kb, at least 6 kb, at least kb, at least 8 kb, at least 8 kb, or at least 10 kb to be divided into two or more fragments or portions, which can each be introduced into a cell or subject via separate vectors, such as multiple AAV. This helps to overcome the limited space available in vectors. In some examples, an endogenous promoter length limits the capability of its corresponding gene to be expressed in an AAV. In some examples, a coding sequence length limits its capability to be expressed in an AAV. In some examples, an endogenous promoter length and is coding sequence length limits their capability to be expressed together in an AAV. The disclosed systems can be used to express such long sequences that have been previously difficult to express in AAV.

In some examples, the target protein to be reconstituted is a protein associated with disease, such as a monogenic disease, recessive genetic disease, a disease caused by a mutation in a large gene (e.g., greater than about 4500 nt, such as those of at least 5 kb, at least 5.5 kb, at least 6 kb, at least kb, at least 8 kb, at least 8 kb, or at least 10 kb), and/or disease caused by a gene (such as a promoter+coding sequence) that exceed AAV's capacity (e.g,. greater than 5000 nt). Examples of such diseases include, but are not limited to, hemophilia A (caused by mutations in the F8 gene, 7 kb coding region), hemophilia B (caused by mutations in the F9 gene), Duchenne muscular dystrophy (caused by mutations in the dystrophin gene, 11 kb coding region), sickle cell anima (caused by mutation in beta globin domain of hemoglobin, which has a promoter of about 3.5 kb), Stargardt disease (caused by mutations in the ABCA4 gene, 6.9 kb coding region), Usher syndrome (caused by a mutation in MYO7A, 7 kb coding region, resulting in hearing loss and visual impairment).

In one example, the target protein to be reconstituted is one that can treat a disease, such as a cancer, such as a cancer of the breast, lung, prostate, liver, kidney, brain, bone, ovary, uterus, skin, or colon. In one example, the therapeutic target protein to be reconstituted is a toxin, such as an AB toxin, such as diphtheria toxin A or pseudomonas exotoxin A, or a form that lacks receptor binding activity (e.g., diphtheria toxin DAB389, DAB486, DT388, DT390, or pseudomonas exotoxin A PE38 or PE40).

In some examples, an RNA sequence encoding the target protein and used in the disclosed methods and systems are codon optimized for expression in a target organism or cell, such as codon optimized for expression in a human, canine, pig, feline, mouse, or rat cell. Thus, in some examples, the RNA coding sequence includes preferred codons (e.g., does not include rare codons with low utilization). Codon optimization can be performed by identifying abundant tRNA levels in the target organism or cells. In some examples, an RNA sequence encoding the protein is de-enriched for cryptic splice donor and acceptor sites to maximize an RNA recombination reaction.

In some examples, a protein is divided into two portions, such as about two equal halves (or other proportions, such as portion A expressing about ⅓ and portion B expressing about ⅔, or portion A expressing about ¼ and portion B expressing about ¾, etc.). However, it is not required that each portion be the same number of nucleotides (or encode the same number of amino acids). In such an example, the method can use two synthetic RNA molecules, one which includes a coding sequence for an N-terminal portion of the protein, and another which includes a coding sequence for a C-terminal portion of the protein. Based on this foundation, one skilled in the art will appreciate that in addition to dividing a protein into two fragments or portions, proteins of interest can be divided or split into more than two fragments, such as three fragments. The design principle of the intronic sequences of three RNA molecules is similar to that of the two, but instead a different pair of dimerization domains for one of the two junctions is utilized. Thus, for example, an N-terminal protein coding sequence is followed by an intronic sequence with a specific binding domain (e.g., first dimerization sequence), the middle coding sequence includes an intronic sequence with a complementary sequence to the first dimerization sequence (second dimerization sequence). The middle coding fragment is followed by another intronic fragment with another dimerization sequence (third dimerization sequence, different from the second dimerization sequence). The third fragment includes the C-terminal coding sequence of the protein, and includes an intronic region with a dimerization sequence (fourth dimerization sequence) complementary to the third dimerization sequence.

In one example, a desired protein is divided into an N-terminal portion and a C-terminal portion (e.g., divided in roughly half, or unequal apportionment, such as ⅓ and ⅔ or ¼ and ¾), which can be reconstituted using the disclosed systems and methods. Referring to FIG. 6A, in such an example, the system includes at least two synthetic nucleic acid molecules 110, 150. Each nucleic acid molecule 110, 150 can be composed of RNA. In some examples, each of 110, 150 is about at least 100 nucleotides/ribonucleotides (nt) in length, such as at least 200, at least 300, at least 500, at least 1000, at least 2000, at least 3000, at least 4000, at least 5000, at least 6000, at least 7000, at least 8000 nt, at least 10,000 nt, such as 200 to 10,000 nt, 200 to 8000 nt, 500 to 5000 nt, or 200 to 1000 nt. The molecules 110, 150 can include natural and/or non-natural nucleotides or ribonucleotides.

Molecule 110 is the 5′-located molecule of the system, as it includes a splice donor 116. Molecule 110 includes from 5′ to 3′, a promoter 112 operably linked to a 5′-fragment of RNA 114 encoding an N-terminal portion of a target protein (which includes a splice junction at its 3′-end). Any promoter 112 (or enhancer) can be used, such as one that utilizes RNA polymerase II, such as a constitutive or inducible promoter. In some examples, promoter 112 is a tissue-specific promoter, such as one constitutively active in muscle tissue (such as skeletal or cardiac), optical tissue (such as retinal tissue), inner ear tissue, liver tissue, pancreatic tissue, lung tissue, skin tissue, bone, or kidney tissue. In some examples, promoter 112 is a cell-specific promoter, such as one constitutively active in a cancer cell, or a normal cell. In some examples, promoter 112 is an endogenous promoter of the target protein expressed, and in some example is long (e.g., at least 2500 nt, at least 3000 nt, at least 4000 nt, at least 5000 nt, or at least 7500 nt). In some examples, promoter 112 is at least about 50 nucleotides/ribonucleotides (nt) in length, such as at least 100, at least 200, at least 300, at least 500, at least 1000, at least 2000, at least 3000, at least 4000, at least 5000, at least 6000, at least 7000, at least 8000 nt, at least 9000 nt, or at least 10,000 nt, such as 50 to 10,000 nt, 100 to 5000 nt, 500 to 5000 nt, or 50 to 1000 nt in length. The splice junction at the 3′ end of the N-terminal coding sequence 114 is an exonic sequence, which can match the consensus sequence found in the target cell or organism into which molecules 110, 150 are introduced. In humans the splice junction sequence is AG (adenine-guanine) or UG (uracil-guanine) at positon-1 and-2 of the 5′ splice site for U2-dependent introns or AG, UG, CU (cytosine-uracil), or UU for U12-dependent introns. Thus, in some examples, the splice junction is 2 nt in length, and the 3′ end of the N-terminal coding portion 114 is AG, UG, CU or UU. In some examples an RNA molecule encoding a portion of a target protein comprises multiple splice junctions, e.g., at the 3′ end of the RNA molecule encoding the N-terminal portion of the target protein, and at the 5′ end of the RNA molecule encoding the C-terminal portion of the target protein. In some examples, these splice junctions may be referred to as a first and second splice junction. In some examples wherein the system comprises more than two RNA molecules, it is understood that the molecules can comprise third, fourth, etc. splice junctions.

The remaining 3′-terminal portion of molecule 110 is intronic, 130. In some examples, intronic sequence 130 is about at least 10 nt, such as at least 20 nt, at least 50 nt, at least 100 nt, at least 250 nt, at least 250 nt, at least 300 nt, at least 400 nt, or at least 500 nt in length, such as 20 to 500, 20 to 250, 20 to 100, 50 to 100, or 50 to 200 nt in length. Immediately following N-terminal coding sequence 114 is a splice donor (SD) 116 (such as a SD consensus sequence, such as a SD human consensus sequence). Thus SD 116 of intronic sequence 130 is 3′ to N-terminal coding sequence 114. SD 116 forms a recognition sequence for the spliceosome components to bind to the RNA molecule. The sequence of SD 116 can be a SD consensus sequence found in the target cell or organism into which molecules 110, 150 are introduced. In some examples, SD 116 is at least 2 nt, such as at least 5 nt, or at least 10 nt in length, such as 2 to 10, 2 to 8, 2 to 5 or 5 to 10 nt. The SD 116 can be used to recruit U2 or U12 dependent splicing machinery. In one example, U2 dependent splicing is used in human cells, and the SD 116 sequence includes or is GUAAGUAUU. In one example, U12 dependent splicing is used in human cells, and the SD 116 sequence includes or is AUAUCCUUUUUA (SEQ ID NO: 137) or GUAUCCUUUUUA (SEQ ID NO: 138).

Intronic sequence 130 optionally includes one or both of a set of splicing enhancer sequences referred to as downstream intronic splice enhancer (DISE) 118 and intronic splice enhancer (ISE) 120, which stimulate action (e.g., increase activity) of the spliceosome. In some examples, intronic sequence 130 includes at least two splicing enhancer sequences, such as at least 3, at least 4, or at least 5 splicing enhancer sequences. Exemplary splicing enhancer sequences include DISE 118 and ISE 120. In some examples, inclusion of one or more splicing enhancer sequences 118, 120 in intronic sequence 130 increases splicing efficiency by at least 20%, at least 30%, at least 40%, at least 50%, at least 75%, at least 80%, at least 90% or at least 95%. Exemplary splicing enhancer sequences that can be used are provided in SEQ ID NOS: 26-136, 151, and 152, as well as GGGTTT, GGTGGT, TTTGGG, GAGGGG, GGTATT, GTAACG, GGGGGTAGG, GGAGGGTTT, GGGTGGTGT TTCAT, CCATTT, TTTTAAA, TGCAT, TGCATG, TGTGTT, CTAAC, TCTCT, TCTGT, TCTTT, TGCATG, CTAAC, CTGCT, TAACC, AGCTT, TTCATTA, GTTAG, TTTTGC, ACTAAT, ATGTTT, CTCTG, GGG, GGG (N)2-4GGG, TGGG, YCAY, UGCAUG, or 3x(G₃₋₆N₁₋₇). In some examples, if DISE 118 is present, can be at least 3 nt, at least 4 nt, at least 5 nt, at least 10 nt, at least 25 nt, at least 50 nt, at least 75 nt, or at least 100 nt in length, such as 3 to 10, 3 to 11, 4 to 11, 5 to 11, 10 to 50, 5 to 100, 10 to 25, 10 to 20, or 20 to 75 nt, the sequence of DISE 118 is or comprises CUCUUUCUUUTCCAUGGGUUGGCU (SEQ ID NO: 134), TGCATG, CTAAC, CTGCT, TAACC, AGCTT, TTCATTA, GTTAG, TTTTGC, ACTAAT, ATGTTT or CTCTG. In some examples, if ISE 120 is present, it can be about at least 3 nt, at least 4 nt,at least 5 nt, at least 10 nt, such as at least 20 nt, at least 25 nt, at least 30 nt, at least 40 nt, or at least 50 nt in length, such as 3 to 10, 3 to 11, 4 to 11, 5 to 11, 10 to 50, 20 to 25, 10 to 25, 10 to 20, or 20 to 40 nt in length. In one example, the sequence of ISE 120 is or comprises GGCUGAGGGAAGGACUGUCCUGGG (SEQ ID NO: 135), GGGUUAUGGGACC (SEQ ID NO: 136), TTCAT, CCATTT, TTTTAAA, TGCAT, TGCATG, TGTGTT, CTAAC, TCTCT, TCTGT, or TCTTT. In some examples, intronic sequence 130 includes at least two, at least 3, or at least 4 ISEs 120.

The SD 116 (and if present also enhancer sequences 118, 120) is followed 3′ by a dimerization domain 122 used to bring the N-terminal coding sequence 114, and C-terminal coding sequence 154 to be combined, together. Intronic sequence 130 portion of molecule 110 can optionally include at the 3′-end a polyadenylation site 124, which terminates transcription of that fragment. In some examples, polyadenylation sequence 124 is a polyA sequence of at least 15 As, such as 15 to 30 or 15 to 20 As.

In some examples, first dimerization domain 122 (and second dimerization domain 154 of molecule 150) includes a plurality of unpaired nucleotides (that is, unpaired within the structure of the molecule 110 itself). Having unpaired nucleotides in the dimerization domain allows the 5′ (or first) dimerization domain 122 and the 3′ (or second) dimerization domain 154 to interact through base pairing. Through this interaction, molecules 110 and 150 are kept in proximity which prompts the spliceosome to recombine the two molecules by joining the N-terminal coding region 114 and the C terminal coding region 164.

In one example, dimerization domain 122 (and 154) includes “hypodiverse sequences,” which contain a limited diversity of nucleotides and are thus unlikely to form stem loops with themselves in the secondary structure of each molecule 110, 150. Such a hypodiverse dimerization domain 122 (and 154) can be a relatively open configuration, independent of the sequences of the RNA encoding the N- and C-terminus of the protein 114, 164. This allows the nucleotides of the first dimerization domain 122 to be available to form base pairs with the corresponding second dimerization domain 154 of molecule 150, allowing subsequent joining of the N-terminal coding sequence 114 and C-terminal coding sequence 164. In some examples, first and second dimerization domain 122, 154 includes hypodiverse sequences interspersed with sequences that can form a stem, which results in local RNA loops that are open and available for basepairing in the absence of pseudoknot formation (FIG. 6B). Exemplary hypodiverse sequences include a repeated series of Us (such as 30 to 500 Us), a repeated series of As (such as 30 to 500 As), a repeated series of Gs (such as 30 to 500 Gs), a repeated series of Cs (such as 30 to 500 Cs), a mixture containing only As and Gs (such as 30 to 500 As and Gs, e.g., AAAGAAGGAA( . . . ) (SEQ ID NO: 149) which can be repeated), a mixture containing only Cs and Us (such as 30 to 500 Cs and Us, e.g., CUUUCUUUUCUU( . . . ) (SEQ ID NO: 150) which can be repeated). Other exemplary hypodiverse sequences include complementary sequences that form helices flanked by hypodiverse sequences.

In some examples, first and second dimerization domain 122, 154 only include purines or only include pyrimidines. In one example, the first dimerization domain 122 only includes purines, while the second dimerization domain 154 only includes pyrimidines. In another example, the first dimerization domain 122 only includes pyrimidines, while the second dimerization domain 154 only includes purines. Due to the inability of purines to pair with themselves (and pyrimidines likewise) these stretches of RNA have an open predicted structure.

In some examples, first and second dimerization domain 122, 154 do not include cryptic splice acceptors that could compete with RNA recombination, such as sequences similar to the splice donor consensus sequence NNNAGGUNNNN (SEQ ID NO: 151) or NNNUGGUNNNN (SEQ ID NO: 152) (wherein N refers to any nucleotide). In some examples, first dimerization domain 122 is no more than 1000 nt, such as no more than 750 nt, or more than 500 nt, such as 6 to 1000 nt, 10 to 1000 nt, 20 to 1000 nt, 30 to 1000 nt, 30 to 750 nt, 30 to 500 nt, 50 to 500 nt, 50 to 100 nt, or 100 to 250 nt. In some examples, first dimerization domain 122 is greater than 50 nt, such as at least 51 nt, at least 100 nt, at least 150 nt, at least 161 nt, or at least 170 nt, such as 51 to 159 nt, 51 to 150 nt, 51 to 120 nt, 51 to 100 nt, or 51 to 70 nt. In some examples, first dimerization domain 122 is greater than 160 nt, such as at least 161 nt, at least 170 nt, at least 180 nt, at least 200 nt, at least 300 nt, at least 400 nt, at least 500 nt, at least 600 nt, at least 700 nt, at least 800 nt, at least 900 nt, or at least 1000 nt, such as 161 to 100 nt, 161 to 500 nt, 161 to 300 nt, 161 to 200 nt, or 161 to 170 nt. In some examples, first dimerization domain 122 is less than 50 nt, such 6 to 49 nt, 6 to 45 nt, 6 to 40 nt, 6 to 30 nt, 6 to 20 nt, or 6 to 10 nt.

In some examples, a dimerization domain is 20 to 160 nt, 50-500 nt, or 500-1000 nt. In some examples, a dimerization domain is about 20 nt to about 160 nt. In some examples, a dimerization domain is about 20 nt to about 40 nt, about 20 nt to about 50 nt, about 20 nt to about 70 nt, about 20 nt to about 90 nt, about 20 nt to about 100 nt, about 20 nt to about 110 nt, about 20 nt to about 120 nt, about 20 nt to about 130 nt, about 20 nt to about 140 nt, about 20 nt to about 150 nt, about 20 nt to about 160 nt, about 40 nt to about 50 nt, about 40 nt to about 70 nt, about 40 nt to about 90 nt, about 40 nt to about 100 nt, about 40 nt to about 110 nt, about 40 nt to about 120 nt, about 40 nt to about 130 nt, about 40 nt to about 140 nt, about 40 nt to about 150 nt, about 40 nt to about 160 nt, about 50 nt to about 70 nt, about 50 nt to about 90 nt, about 50 nt to about 100 nt, about 50 nt to about 110 nt, about 50 nt to about 120 nt, about 50 nt to about 130 nt, about 50 nt to about 140 nt, about 50 nt to about 150 nt, about 50 nt to about 160 nt, about 70 nt to about 90 nt, about 70 nt to about 100 nt, about 70 nt to about 110 nt, about 70 nt to about 120 nt, about 70 nt to about 130 nt, about 70 nt to about 140 nt, about 70 nt to about 150 nt, about 70 nt to about 160 nt, about 90 nt to about 100 nt, about 90 nt to about 110 nt, about 90 nt to about 120 nt, about 90 nt to about 130 nt, about 90 nt to about 140 nt, about 90 nt to about 150 nt, about 90 nt to about 160 nt, about 100 nt to about 110 nt, about 100 nt to about 120 nt, about 100 nt to about 130 nt, about 100 nt to about 140 nt, about 100 nt to about 150 nt, about 100 nt to about 160 nt, about 110 nt to about 120 nt, about 110 nt to about 130 nt, about 110 nt to about 140 nt, about 110 nt to about 150 nt, about 110 nt to about 160 nt, about 120 nt to about 130 nt, about 120 nt to about 140 nt, about 120 nt to about 150 nt, about 120 nt to about 160 nt, about 130 nt to about 140 nt, about 130 nt to about 150 nt, about 130 nt to about 160 nt, about 140 nt to about 150 nt, about 140 nt to about 160 nt, or about 150 nt to about 160 nt. In some examples, a dimerization domain is about 20 nt, about 40 nt, about 50 nt, about 70 nt, about 90 nt, about 100 nt, about 110 nt, about 120 nt, about 130 nt, about 140 nt, about 150 nt, or about 160 nt. In some examples, a dimerization domain is at least about 20 nt, about 40 nt, about 50 nt, about 70 nt, about 90 nt, about 100 nt, about 110 nt, about 120 nt, about 130 nt, about 140 nt, or about 150 nt. In some examples, a dimerization domain is at most about 40 nt, about 50 nt, about 70 nt, about 90 nt, about 100 nt, about 110 nt, about 120 nt, about 130 nt, about 140 nt, about 150 nt, or about 160 nt.

In some examples, a dimerization domain is about 50 nt to about 500 nt. In some examples, a dimerization domain is about 50 nt to about 100 nt, about 50 nt to about 150 nt, about 50 nt to about 200 nt, about 50 nt to about 250 nt, about 50 nt to about 300 nt, about 50 nt to about 350 nt, about 50 nt to about 400 nt, about 50 nt to about 500 nt, about 100 nt to about 150 nt, about 100 nt to about 200 nt, about 100 nt to about 250 nt, about 100 nt to about 300 nt, about 100 nt to about 350 nt, about 100 nt to about 400 nt, about 100 nt to about 500 nt, about 150 nt to about 200 nt, about 150 nt to about 250 nt, about 150 nt to about 300 nt, about 150 nt to about 350 nt, about 150 nt to about 400 nt, about 150 nt to about 500 nt, about 200 nt to about 250 nt, about 200 nt to about 300 nt, about 200 nt to about 350 nt, about 200 nt to about 400 nt, about 200 nt to about 500 nt, about 250 nt to about 300 nt, about 250 nt to about 350 nt, about 250 nt to about 400 nt, about 250 nt to about 500 nt, about 300 nt to about 350 nt, about 300 nt to about 400 nt, about 300 nt to about 500 nt, about 350 nt to about 400 nt, about 350 nt to about 500 nt, or about 400 nt to about 500 nt. In some examples, a dimerization domain is about 50 nt, about 100 nt, about 150 nt, about 200 nt, about 250 nt, about 300 nt, about 350 nt, about 400 nt, or about 500 nt. In some examples, a dimerization domain is at least about 50 nt, about 100 nt, about 150 nt, about 200 nt, about 250 nt, about 300 nt, about 350 nt, or about 400 nt. In some examples, a dimerization domain is at most about 100 nt, about 150 nt, about 200 nt, about 250 nt, about 300 nt, about 350 nt, about 400 nt, or about 500 nt.

In some examples, the sequence of first and second dimerization domains 122 and 154 are determined by in silico structure prediction screening (e.g., RNA folding structure prediction is used to screen a library of possible dimerization domain sequences; sequences with a large proportion of unpaired nucleotides in both the dimerization domain and the corresponding anti-dimerization domain are selected), hypodiverse nucleotide design (e.g., dimerization domain designed to include a stretch of hypodiverse sequence, such as a repeat sequence of only U, only A, only C, only G, only R (G and A), or only Y (U and C), the sequence cannot fold onto itself), or empirical screening (e.g., a library of dimerization domains and corresponding anti-dimerization domains are synthesized and screened for maximal recombination efficiency).

In some examples, the sequence of first and second dimerization domains 122, 154 are designed to contain complementary RNA hairpin structures (also called stem loops) that can form strong kissing loop interactions with their counter parts. In some examples, kissing loops are used when three or more dimerization domains are used to join three or more portions of a coding sequence, such as four or more or five or more dimerization domains, such as 3, 4, 5, 6, 7, 8, 9 or 10 dimerization domains (e.g., FIG. 6E). Each hairpin loop (or stem loop) of a kissing loop is composed of at least two complementary sequences (e.g., form a stem) separated by a region of non-complementary sequence (e.g., form a loop). In some examples, a dimerization domain can be composed of 1 or more (such as at least 2, at least 3, at least 4, or at least 5, such as 2, 3, 4, 5, 6, 7, 8, 9, 10, 11, 12, 13, 14, 15, 16, 17, 18, 19, or 20) loops. In some examples with multiple loops, all or some of the loops can be repeated. In some examples with multiple loops, all or some loops can be different In some examples, each complementary sequence is about 4 to 100 nt, which are separated by a loop of about 3 to 20 nt. Base-pairing between the two complementary sequences results in a helix (or stem), for example of at least 4 bp, at least 5 bp, at least 10 bp, at least 20 bp, at least 30 bp, at least 40 bp, at least 50 bp, at least 75 bp, at least 90 bp, or at least 100 bp, such as 4 to 100 bp, 5 to 75 bp, or 10 to 50 bp. In some examples, the loop portion is at least 3 nt, at least 5 nt, at least 10 nt, at least 15 nt, or at least 20 nt, such as 3 to 20 nt, 5 to 15 nt or 5 to 10 nt, wherein the loop is not base paired. Complementary sequences between two hairpin loops result in base pairing, and generation of a kissing loop/kissing stem loop interaction. In some examples, the complementary sequences between the two hairpin loops occurs between at least 3 nucleotides of one loop with at least 3 nucleotides of a second loop, such as at least 4, at least 5, at least 6, at least 7, at least 8, at least 9, at least 10, at least 11, at least 12, at least 13, at least 14, at least 15, at least 16, at least 17, at least 19, or at least 20 nt (such as 3, 4, 5, 6, 7, 8, 9, 10, 11, 12, 13, 14, 15, 16, 17, 18, 19 or 20) of the first loop, with at least 4, at least 5, at least 6, at least 7, at least 8, at least 9, at least 10, at least 11, at least 12, at least 13, at least 14, at least 15, at least 16, at least 17, at least 19, or at least 20 nt (such as 3, 4, 5, 6, 7, 8, 9, 10, 11, 12, 13, 14, 15, 16, 17, 18, 19 or 20) of the second loop. In some examples, the complementary sequences between the two hairpin loops occurs between at least 15%, at least 20%, at least 30%, at least 40%, at least 50%, at least 60%, at least 70%, at least 80%, at least 90%, at least 95%, or 100% of the total loop sequence.

In some instances, the stems of the kissing loops are chosen to base pair in trans between the two RNA molecules. In such an example, after forming a kissing loop interaction of one hairpin loop on one molecule with another hairpin loop on a second molecule, the respective stem (or helix) regions of the initial hairpin loops can base pair in trans between the two RNA molecules through strand replacement/invasion and extended duplex formation. In some examples, within the initial loop sequence, up to about 85% of nucleotides can remain unpaired after extended duplex formation (e.g.. about 15% of the nt are paired between the two loops). In some examples, the kissing loop is based on the HIV-1 DIS loop (SEQ ID NOS: 139 and 140, FIG. 17A), and includes two A nucleotides on the 5′ side of 6 nucleotides of complementary sequence, followed by one A nucleotide on the 3′ side (e.g., AANNNNNNA where N can be any of A, U, G, or C). In some examples, the kissing loop is based on the HIV-2 kissing loop dimerization domain (SEQ ID NOS: 141 and 142, FIG. 17B), and includes a G and an A nucleotide on the 5′ side of six nucleotides of complementary sequence followed by three A nucleotides on the 3′ side (e.g., GANNNNNNAAA (SEQ ID NO: 153) where N can be A, U, G, or C).

In one configuration, extended duplex formation is favored by inclusion of mismatches in the initial stems that result in higher percentage of matching in the extended duplex. Thus, in some examples, the helix or stem region of a hairpin loop can contain up to 30% of base pairs that are not paired initially (e.g., no more than 30%, no more than 20%, no more than 15%, no more than 10%, no more than 5%, or no more than 1%, such as 1 to 30%, 5 to 30%, 10 to 30%, or 25 to 30% of base pairs are not paired initially). These regions of non-pairing can form bulges, mismatches, or internal loops.

In addition to an interaction of two hairpin loops (kissing loop interaction), other forms of loop interactions can be utilized for the first and second dimerization domains 122, 154. In one example the loops are bulges, where one strand of a base paired helix contains one or more nucleotides that bulge out from the stem structure. Exemplary bulges are at least 1 nt, at least 2 nt, at least 3 nt, at least 4 nt, at least 5 nt, at least 10 nt or at least 20 nt, such as 1 to 20 nt, 1 to 15 nt, 1 to 10 nt, or 5 to 10 nt, or 1, 2, 3, 4, 5, 6, 7, 8, 9, 10, 11, 12, 13, 14, 15, 16, 17, 18, 19 or 20 nt. In one example the loops are internal loops, for example, where 1 or more nucleotides in a helix are mismatched, resulting in a helix interrupted by an internal loop at the positions of mismatch. In some examples the helix is at least 4 nt on each of the strands (e.g., at least 5 nt, at least 10 nt, at least 20 nt, at least 30 nt, at least 40 nt, at least 50 nt, at least 75 nt, at least 90 nt, or at least 100 nt, such as 4 to 100 nt, 5 to 75 nt, or 10 to 50 nt. such as 4 to 100 nt), on either side of the internal loop that is at least 1 nt (e.g., at least 2 nt, at least 3 nt, at least 4 nt, at least 5 nt, at least 10 nt or at least 20 nt, such as 1 to 20 nt, 1 to 15 nt, 1 to 10 nt, or 5 to 10 nt, or 1, 2, 3, 4, 5, 6, 7, 8, 9, 10, 11, 12, 13, 14, 15, 16, 17, 18, 19 or 20 nt on each of the strands). In one example the loops are multi-branched loops, wherein three helices or stems from a triangle with one or more unpaired nucleotides connecting the three helices. In some examples, each of the helices is at least 4 bp (e.g., at least 5 bp, at least 10 bp, at least 20 bp, at least 30 bp, at least 40 bp, at least 50 bp, at least 75 bp, at least 90bp, or at least 100 bp, such as 4 to 100 bp, 5 to 75 bp, or 10 to 50 bp), and the unpaired nucleotides that form the triangle are at least 3 nt (e.g., at least 4 nt, at least 5 nt, at least 10 nt, at least 20, at least 15, at least 30, at least 40, at least 50, or at least 60 nt, such as 3 to 60 nt, 3 to 30 nt, 3 to 25 nt, or 5 to 20 nt, such as 1, 2, 3, 4, 5, 6, 7, 8, 9, 10, 11, 12, 13, 14, 15, 16, 17, 18, 19, 2, 25, 30, 35, 40, 45, 50, 55 or 60 nucleotides). A kissing interaction can occur between any two of these types of loops (e.g., between two or more binding domains that each include one or more helices). In some examples, helices within one dimerization domain (e.g., first dimerization domain 122) have a direct counterpart in the other binding domain (e.g., second dimerization domain 154) to allow for extended duplex formation after initial loop kissing interaction. In some examples, dimerization domains containing helices to generate loops, form a single kissing stem loop upon interaction between the two or more dimerization domains (e.g., 122, 154 of FIG. 6A). In some examples, dimerization domains containing helices form multiple loops for kissing loop interactions upon interaction between the two or more dimerization domains (e.g., 122, 154 of FIG. 6A). In some examples, one or more dimerization domains (e.g., 122 of FIG. 6A) contain helices destabilized by the inclusion of bulges, single base bulges, mismatches or internal loops, or G-U wobble pairs, but match to the other binding domain (e.g., 154 of FIG. 6A), to favor extended duplex formation after initial kissing/pairing. In some examples, one or more dimerization domains (e.g., 122 of FIG. 6A) contain destabilized helices, which when stabilized (e.g., theophylline switch kissing loop) expose a loop that can interact with a second dimerization domain (e.g., 122 of FIG. 6A) via loop-loop interactions (e.g., kissing/pairing).

In some examples these stem loops contain at least 10 nt, such as at least 20 nt, at least 25 nt, at least 50 nt, at least 75 nt, or at least 100 nt in length, such as 10 to 50, 20 to 25, 10 to 100, 10 to 20, or 20 to 40 nt in length. Each dimerization domain can contain at least 1 individual stem loop, such as at least 2, at least 5, at least 10, at least 15, or at least 20, such as 1 to 20, 2 to 5 or 1 to 10 individual stem loops.

In some examples, 3 to 10 portions of a coding sequence are joined by 2 to 9 kissing loops, e.g., 3 portions are joined by 2 kissing loops, 4 portions are joined by 3 kissing loops, etc., wherein each of the 2 to 9 kissing loops are different. In some examples, a kissing loop comprises multiple stem loops, e.g., 2 to 20 stem loops. In some examples, each of the multiple stem loops in the kissing loop are the same. In some examples, each of the multiple stem loops in the kissing loop are different. In some examples, a dimerization domain comprises 1 to 20 stem loops. In some examples, a dimerization domain comprises 1 stem loop to 20 stem loops. In some examples, a dimerization domain comprises 1 stem loop to 2 stem loops, 1 stem loop to 3 stem loops, 1 stem loop to 4 stem loops, 1 stem loop to 5 stem loops, 1 stem loop to 6 stem loops, 1 stem loop to 7 stem loops, 1 stem loop to 8 stem loops, 1 stem loop to 9 stem loops, 1 stem loop to 10 stem loops, 1 stem loop to 15 stem loops, 1 stem loop to 20 stem loops, 2 stem loops to 3 stem loops, 2 stem loops to 4 stem loops, 2 stem loops to 5 stem loops, 2 stem loops to 6 stem loops, 2 stem loops to 7 stem loops, 2 stem loops to 8 stem loops, 2 stem loops to 9 stem loops, 2 stem loops to 10 stem loops, 2 stem loops to 15 stem loops, 2 stem loops to 20 stem loops, 3 stem loops to 4 stem loops, 3 stem loops to 5 stem loops, 3 stem loops to 6 stem loops, 3 stem loops to 7 stem loops, 3 stem loops to 8 stem loops, 3 stem loops to 9 stem loops, 3 stem loops to 10 stem loops, 3 stem loops to 15 stem loops, 3 stem loops to 20 stem loops, 4 stem loops to 5 stem loops, 4 stem loops to 6 stem loops, 4 stem loops to 7 stem loops, 4 stem loops to 8 stem loops, 4 stem loops to 9 stem loops, 4 stem loops to 10 stem loops, 4 stem loops to 15 stem loops, 4 stem loops to 20 stem loops, 5 stem loops to 6 stem loops, 5 stem loops to 7 stem loops, 5 stem loops to 8 stem loops, 5 stem loops to 9 stem loops, 5 stem loops to 10 stem loops, 5 stem loops to 15 stem loops, 5 stem loops to 20 stem loops, 6 stem loops to 7 stem loops, 6 stem loops to 8 stem loops, 6 stem loops to 9 stem loops, 6 stem loops to 10 stem loops, 6 stem loops to 15 stem loops, 6 stem loops to 20 stem loops, 7 stem loops to 8 stem loops, 7 stem loops to 9 stem loops, 7 stem loops to 10 stem loops, 7 stem loops to 15 stem loops, 7 stem loops to 20 stem loops, 8 stem loops to 9 stem loops, 8 stem loops to 10 stem loops, 8 stem loops to 15 stem loops, 8 stem loops to 20 stem loops, 9 stem loops to 10 stem loops, 9 stem loops to 15 stem loops, 9 stem loops to 20 stem loops, 10 stem loops to 15 stem loops, 10 stem loops to 20 stem loops, or 15 stem loops to 20 stem loops. In some examples, a dimerization domain comprises 1 stem loop, 2 stem loops, 3 stem loops, 4 stem loops, 5 stem loops, 6 stem loops, 7 stem loops, 8 stem loops, 9 stem loops, 10 stem loops, 15 stem loops, or 20 stem loops. In some examples, a dimerization domain comprises at least 1 stem loop, 2 stem loops, 3 stem loops, 4 stem loops, 5 stem loops, 6 stem loops, 7 stem loops, 8 stem loops, 9 stem loops, 10 stem loops, or 15 stem loops. In some examples, a dimerization domain comprises at most 2 stem loops, 3 stem loops, 4 stem loops, 5 stem loops, 6 stem loops, 7 stem loops, 8 stem loops, 9 stem loops, 10 stem loops, 15 stem loops, or 20 stem loops.

Other mechanisms can be used to allow the two or more dimerization domains (e.g., 122, 154 of FIG. 6A) to bind or interact with one another sufficient for recombination of the coding sequences to occur. In some examples, the two or more dimerization domains (e.g., 122, 154 of FIG. 6A) are nucleic acid aptamers (such as RNA aptamers) that can interact with one another, for example through a non-base pairing interaction, or can bind to a common molecule (e.g., protein, ATP, metal ion, co-factor, or synthetic ligand). In some examples, two or more dimerization domains (e.g. 122, 154 of FIG. 6A) do not hybridize to one another, but can both (or all) hybridize to the same bridge nucleic acid molecule. In some examples, such a bridge nucleic acid molecule can be exogenously provided to the cells, tissues, or organism. In some examples, such a bridge nucleic acid molecule can be a DNA or RNA sequence inside the cell, such as a transcript or genomic locus. In some examples, the two or more dimerization domains (e.g., 122, 154 of FIG. 6A) are sequences that can interact with one another, for example through a non-base pairing interaction.

Molecule 150 is the 3′-located molecule, and includes a splice acceptor (SA) 162 and a second dimerization domain 154. Molecule 150 includes from 5′ to 3′, a promoter 152 followed by intronic sequence 170. Promoter 152 can be is operably linked to intronic sequence 170. Any promoter 152 can be used, such as a constitutive or inducible promoter. In some examples, promoter 152 is a tissue-specific promoter, such as one constitutively active in muscle tissue (such as skeletal or cardiac), optical tissue (such as retinal tissue), inner ear tissue, liver tissue, pancreatic tissue, lung tissue, skin tissue, bone, or kidney tissue. In some examples, promoter 112 is a cell-specific promoter, such as one constitutively active in a cancer cell, or a normal cell. In some examples, promoter 112 is an endogenous promoter of te target protein expressed, and in some example is long (e.g., at least 2500 nt, at least 3000 nt, at least 4000 nt, at least 5000 nt, or at least 7500 nt). In some examples, promoter 112 is at least about 50 nucleotides/ribonucleotides (nt) in length, such as at least 100, at least 200, at least 300, at least 500, at least 1000, at least 2000, at least 3000, at least 4000, at least 5000, at least 6000, at least 7000, at least 8000 nt, at least 9000 nt, or at least 10,000 nt, such as 50 to 10,000 nt, 100 to 5000 nt, 500 to 5000 nt, or 50 to 1000 nt in length. In some examples promoter 112 and promoter 152 are the same promoter. In other examples, promoter 112 and promoter 152 are the different promoters.

The intronic sequence 170 includes a second dimerization domain 154, optional ISE 156, branching point 158, polypyrimidine tract 160, followed by a splice acceptor sequence 162. In some examples, intronic sequence 130 is about at least 10 nt, such as at least 20 nt, at least 30 nt, at least 50 nt, at least 100 nt, at least 250 nt, at least 250 nt, at least 300 nt, at least 400 nt, or at least 500 nt in length, such as 20 to 500, 20 to 250, 20 to 100, 50 to 100, 30 to 500, or 50 to 200 nt in length.

Second dimerization domain 154 has a sequence that is the reverse complement of first dimerization domain 122 sequence of molecule 110. Thus, same design features and considerations of first dimerization domain 122 discussed above also apply to second dimerization domain 154. For example, in some examples the second dimerization domain 154 contains a stem loop that can form a kissing loop interaction the first dimerization domain 122. In some examples, second dimerization domain 154 does not include cryptic splice acceptors (e.g., NNNAGGUNNN; SEQ ID NO: 143) that could compete with RNA recombination. In some example, second dimerization domain 154 has a hypodiverse sequence. In some examples, second dimerization domain 154 is no more than 1000 nt, such as no more than 750 nt, or more than 500 nt, such as 30 to 1000 nt, 30 to 750 nt, 30 to 500 nt, 50 to 500 nt, 50 to 100 nt, or 100 to 250 nt. In some examples, second dimerization domain 154 is greater than 50 nt, such as at least 51 nt, at least 100 nt, at least 150 nt, at least 161 nt, or at least 170 nt, such as 51 to 159 nt, 51 to 150 nt, 51 to 120 nt, 51 to 100 nt, or 51 to 70 nt. In some examples, second dimerization domain 154 is greater than 160 nt, such as at least 161 nt, at least 170 nt, at least 180 nt, at least 200 nt, at least 300 nt, at least 400 nt, at least 500 nt, at least 600 nt, at least 700 nt, at least 800 nt, at least 900 nt, or at least 1000 nt, such as 161 to 100 nt, 161 to 500 nt, 161 to 300 nt, 161 to 200 nt, or 161 to 170 nt. In some examples, second dimerization domain 154 is less than 50 nt, such 6 to 49 nt, 6 to 45 nt, 6 to 40 nt, 6 to 30 nt, 6 to 20 nt, or 6 to 10 nt.

3′-to second dimerization domain 154 is an optional ISE 156, branch point sequence 158 (such as a branch point consensus sequence), polypyrimidine tract 160, followed by a splice acceptor sequence 162. ISE 156, like ISE 120 and DISE 118 of molecule 110, stimulates the spliceosome to catalyze the recombination reaction. In some examples, intronic sequence 150 includes at least two ISE 156, such as at least 3, at least 4, or at least 5 ISEs 156. Exemplary splicing enhancer sequences include ISE 156. In some examples, inclusion of one or more splicing enhancer sequences 156 in intronic sequence 150 increases recombination or splicing efficiency by at least 10%, at least 20%, at least 30%, at least 40%, or at least 50%. Exemplary splicing enhancer sequences that can be used are provided in SEQ ID NOS: 26-136, 151, and 152, as well as GGGTTT, GGTGGT, TTTGGG, GAGGGG, GGTATT, GTAACG, GGGGGTAGG, GGAGGGTTT, GGGTGGTGT TTCAT, CCATTT, TTTTAAA, TGCAT, TGCATG, TGTGTT, CTAAC, TCTCT, TCTGT, TCTTT, TGCATG, CTAAC, CTGCT, TAACC, AGCTT, TTCATTA, GTTAG, TTTTGC, ACTAAT, ATGTTT, CTCTG, GGG, GGG (N)2-4GGG, TGGG, YCAY, UGCAUG, or 3x(G₃₋₆N₁₋₇). In some examples, if ISE 156 is present, it can be about least 3 nt, at least 4 nt, at least 5 nt, at least 10 nt, such as at least 20 nt, at least 25 nt, at least 30 nt, at least 40 nt, or at least 50 nt in length, such as 3 to 10, 3 to 11, 4 to 11, 5 to 11, 10 to 50, 20 to 25, 10 to 25, 10 to 20, or 20 to 40 nt in length. In one example, the sequence of ISE 156 is or comprises GGCUGAGGGAAGGACUGUCCUGGG (SEQ ID NO: 135), GGGUUAUGGGACC (SEQ ID NO: 136), TTCAT, CCATTT, TTTTAAA, TGCAT, TGCATG, TGTGTT, CTAAC, TCTCT, TCTGT, or TCTTT. In some examples ISE 120 and ISE 156 are the same sequence. In other examples, ISE 120 and ISE 156 are the different sequences.

3′-to second dimerization domain 154 (and ISE 156 if present) is branch point sequence 158 (such as a branch point consensus sequence), a polypyrimidine tract 160, followed by a splice acceptor sequence 162 (such as a splice acceptor consensus sequence). The sequence of branch point 158 is based on the consensus sequence of the species of the target cell or organism. For example, for human splicing, the consensus sequence can include or be YUNAY. Thus, a sequence that it uses can be CUAAC for U2-dependent introns, or for U12-dependent introns UUUUCCUUAACU (SEQ ID NO: 144).

Polypyrimidine tract 160 includes C, U, or both C and U nucleotides, such as CnUy, wherein n+y is greater than or equal to 10 nucleotides, and can include nucleotides −3 to −22 relative to the 3′-splice junction. In some examples, polypyrimidine tract 160 includes at least 80% Y nucleotides (i.e., U, C, or both U and C). In some examples, polypyrimidine tract 160 is a polyC or polyU sequence. In some examples, polypyrimidine tract 160 is a polyU sequence of at least 15 Us, such as 15 to 30 or 15 to 20 Us. Branch point 158 and polypyrimidine tract 160 are essential splicing components. The sequence of SA 162 can be based on the consensus sequence of the species of the target cell or organism. For example, in humans, the SA sequence can be AG in positions −1 and −2relative to the 3′-splice site for U2-dependnet introns and AC or AG for U12-dependnet introns. Thus, in some examples, SA 162 can be 2 nt in length, such as AG or AC.

Immediately following SA 162 is an exonic sequence which includes RNA sequence encoding a C-terminal portion of a target protein 164 having a splice junction at its 5′end. The splice junction at the 5′end of RNA sequence encoding a C-terminal portion of a target protein 164, that can match the consensus sequence found in the target cell or organism into which molecules 110, 150 are introduced. In some example splice junction can be GA or GU at positon +1 and +2 of the 3′ splice site for U2-dependent introns or GU or AU for U12-dependent introns. Thus, in some examples, the splice junction is 2 nt in length, and the 5′ end of the C-terminal coding portion 164 is GA, GU, or AU.

The exonic sequence following intronic portion 170 of molecule 150 includes a second coding portion (e.g., half) of the target protein, e.g., the C terminal fragment 164, and optional polyadenylation sequence 166. Thus, molecule 150 includes RNA sequence 164 encoding a C-terminal portion of a target protein. The 3′-end of molecule 150 optionally includes a polyadenylation sequence 166, which promotes the assembly of the spliceosome. In some examples, polyadenylation sequence 166 is a polyA sequence of at least 15 As, such as 15 to 30 or 15 to 20 As. In some examples polyadenylation sequence 166 and polyadenylation sequence 124 are the same sequence. In other examples, polyadenylation sequence 166 and polyadenylation sequence 124 are the different sequences.

In some examples, the N-terminal coding region 114 and/or the C terminal coding region 164 is a native coding sequence. For example, the coding sequence is one that is found in the cell or organism into which the disclosed system is introduced. (e.g., a human coding sequence when introduced into a human cell or subject). In some examples, the N-terminal coding region 114 and/or the C terminal coding region 164 is codon optimized relative to a native coding sequence, for example to maximize tRNA availability, or to de-enrich for cryptic splice sites (e.g., to reduce or avoid incorrect splicing and promote the correct junction formation). In some examples, a portion of the N-terminal coding region 114 and/or the C terminal coding region 164 is codon optimized relative to a native coding sequence, for example the about 200 nt adjacent to each junction (e.g., the 3′-end of 114, and the 5′end of 164) can be codon optimized or altered to contain exonic splice enhancer sites (ESE) (which would bind SR proteins). For example, the coding sequence can be one not found in the cell or organism into which the disclosed system is introduced. (e.g., a human coding sequence when introduced into a mouse cell or subject).

In some examples, the N-terminal coding region 114 and/or the C terminal coding region 164 include an intron that is either natural or synthetic in nature and contains both a splice donor and acceptor site. For example, an intron embedded inside the to the coding sequence to be expressed can be included upstream (e.g., about 200 nt upstream) of sequence 116, inside the N-terminal coding region 114, an intron embedded inside the coding sequence to be expressed can be included downstream (e.g., about 200 nt downstream) of the sequence 162 and inside the C-terminal coding region 164, or both. Inclusion of such introns can be used to stimulate splicing machinery attachment to the trans-splicing intron donor and acceptor. In some examples, such (stimulatory-)introns could be derived from the host in which 110 and 150 are expressed. In some examples, such (stimulatory)-introns could be derived from other organisms, or viral in origin, or synthetic in origin.

In some examples, inclusion of a sequence to stabilize the RNA (e.g., placed between 164and 166 in the 3′ untranslated region of 150 in FIG. 6A) can increase expression efficiency of the recombined product by at least 25%, at least 30%, at least 40%, at least 50%, at least 60%, or at least 75%, such as 25 to 95%, 25 to 75%, 25 to 60%, 25 to 50%, 40 to 95%, 40 to 60%, or 50 to 60%. In some examples, woodchuck post-transcriptional regulatory element (WPRE) or truncations thereof (e.g. WPRE3) are included in the 3′-UTR as a stabilizing element to enhance recombined product expression efficiency. In some example a WPRE sequence has at least 80%, at least 85%, at least 90%, at least 95%, or 100% sequence identity to nt 1093 to 1684 of GenBank accession no. J04514 or to the 247 bp sequence of WPRE3.

As shown in FIG. 6C, interaction and hybridization (base pairing) between first dimerization domain 122 of molecule 110 and second dimerization domain 154 of molecule 150, allows the spliceosome components to recombine N-terminal coding sequence 114 and C-terminal coding sequence 164. Specifically the 3′ end of the N terminal protein coding sequence 114 is fused to the 5′ end of the C terminal protein sequence 164 as a seamless junction between the two portions.

FIG. 6D shows a schematic of a system wherein a target protein is divided into three portions, an N-terminal, middle, and C-terminal portion (wherein each portion can be similar or different in size). One skilled in the art will appreciate that a protein can thus be divided into any number of desired segments or portions, and an appropriate number of molecules designed using the information provided herein. In such an example, the system includes at least three synthetic nucleic acid molecules 110, 200, and 150, wherein molecule 110 includes RNA molecule 114 which encodes the N-terminal portion of the protein, molecule 200 includes RNA molecule 216 which encodes the middle portion of the protein, and molecule 150 includes RNA molecule 164 which encodes the C-terminal portion of the protein,. Each nucleic acid molecule 110, 200, 150 can be composed of RNA. In some examples, each of 110, 200, 150 is at least about 100 nucleotides/ribonucleotides (nt) in length, such as at least 200, at least 300, at least 500, at least 1000, at least 2000, at least 3000, at least 4000, at least 5000, at least 6000, at least 7000, or at least 8000 nt, such as 200 to 10,000 nt, 200 to 8000 nt, 500 to 5000 nt, or 200 to 1000 nt. The molecules 110, 150, 200 can include natural and/or non-natural nucleotides or ribonucleotides. In addition to using two (or more) orthogonal dimerization domains, one of the two introns can be a U2-type intron and the second intron can be a U12-type intron. Splice donor and acceptors of U2 and U12 dependent introns show minimal cross reactivity since the consensus recognition sequences between the two types of introns are different. Both strategies (i.e., the orthogonal dimerization domains, and the U2 vs U12 type introns) promote recombination of the three fragments in the correct order (e.g., to avoid the first fragment to directly join up to the last fragment and to avoid the middle fragment circularizing onto itself).

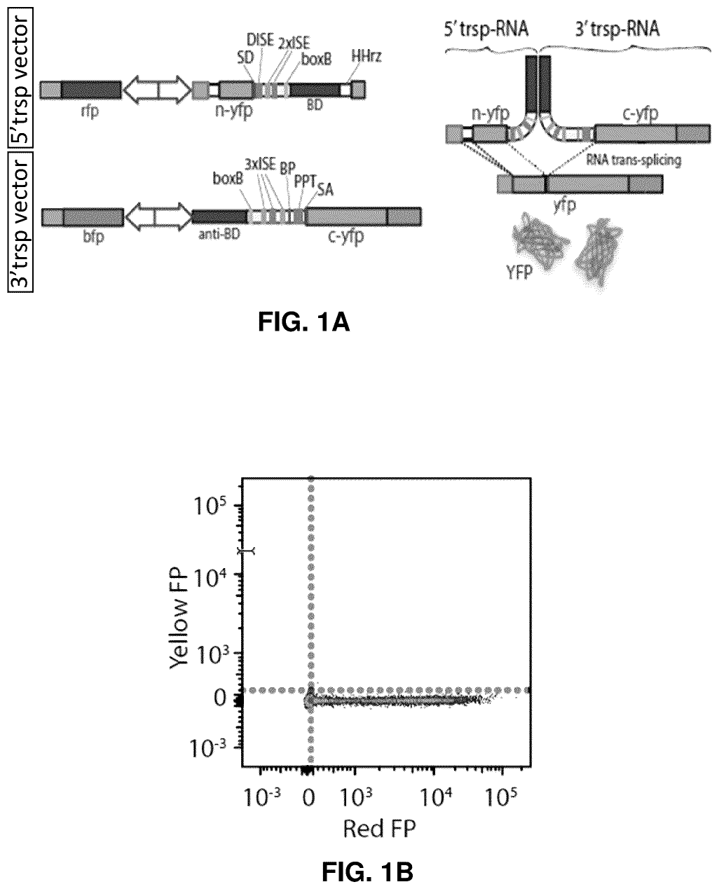

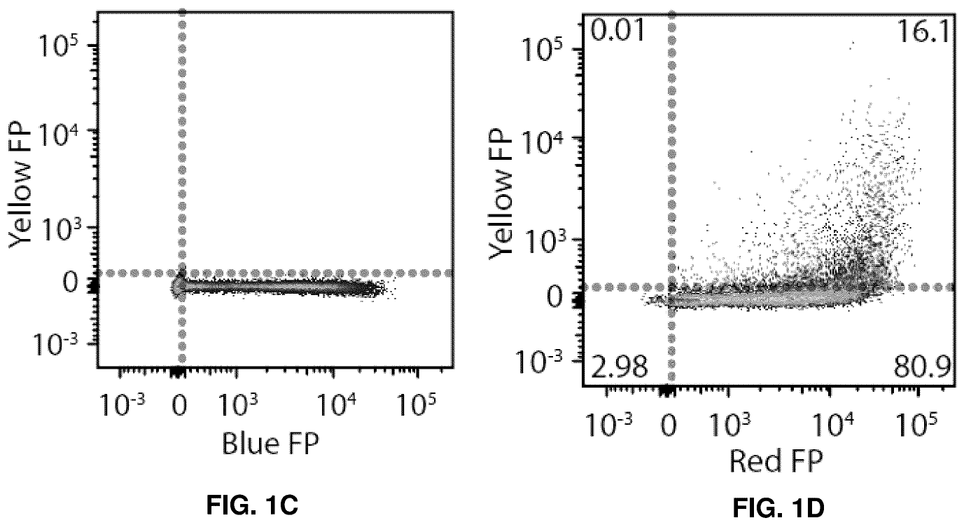

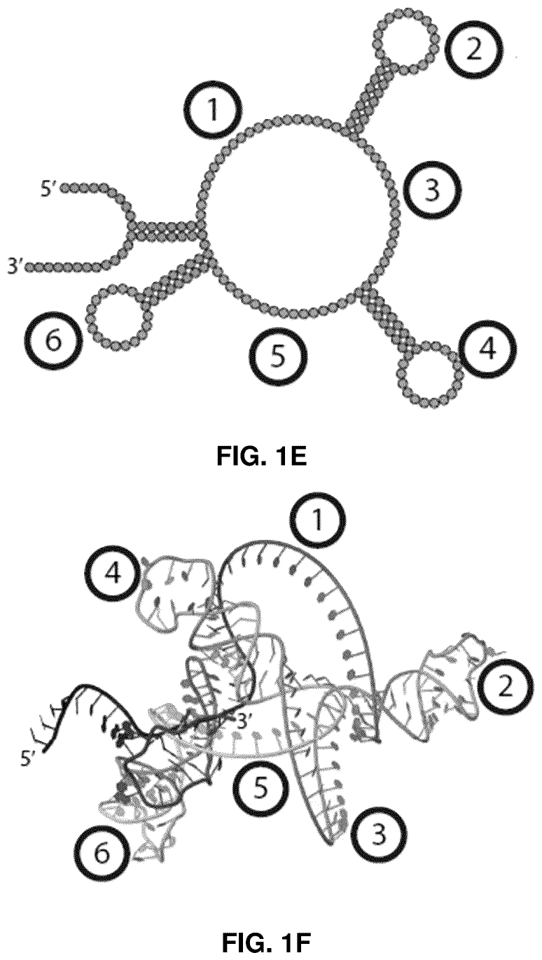

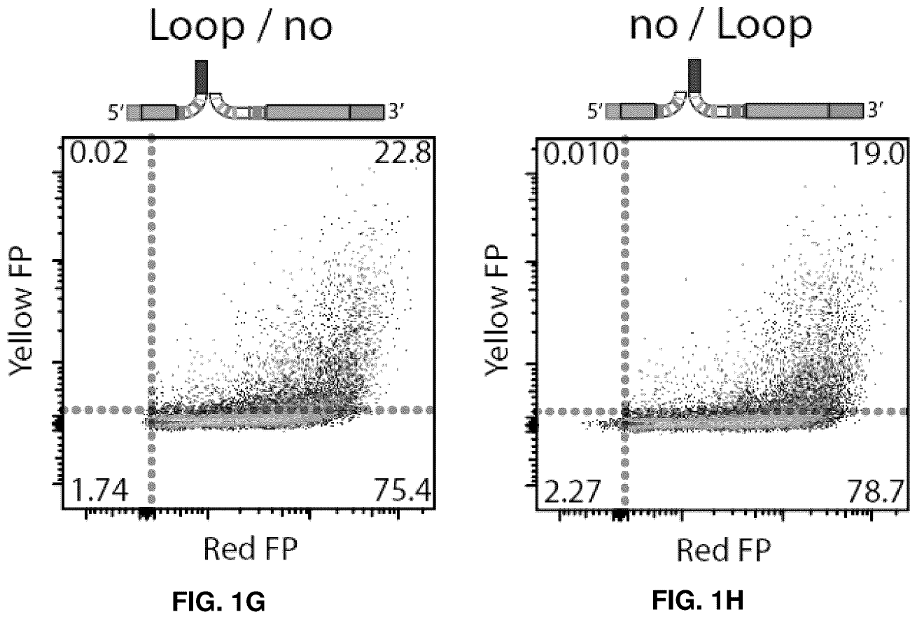

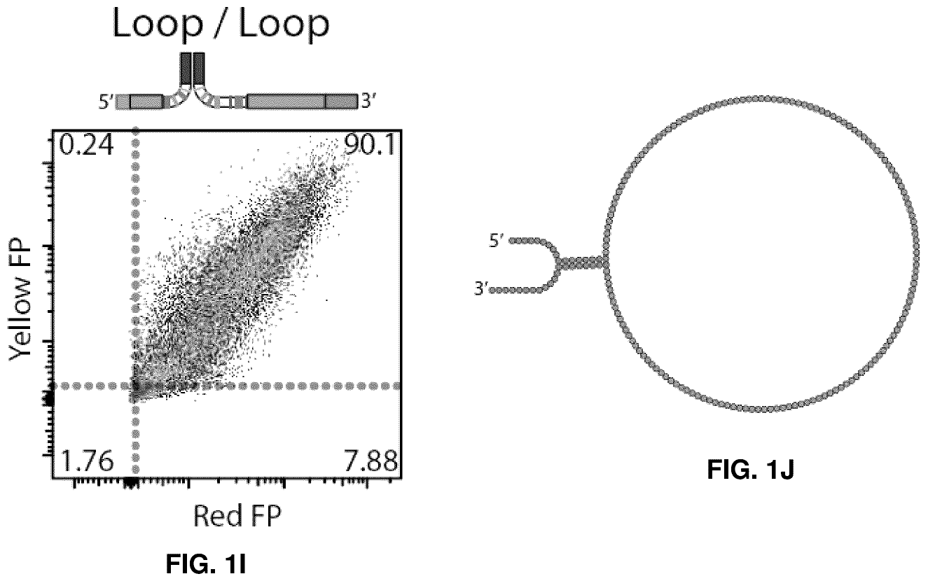

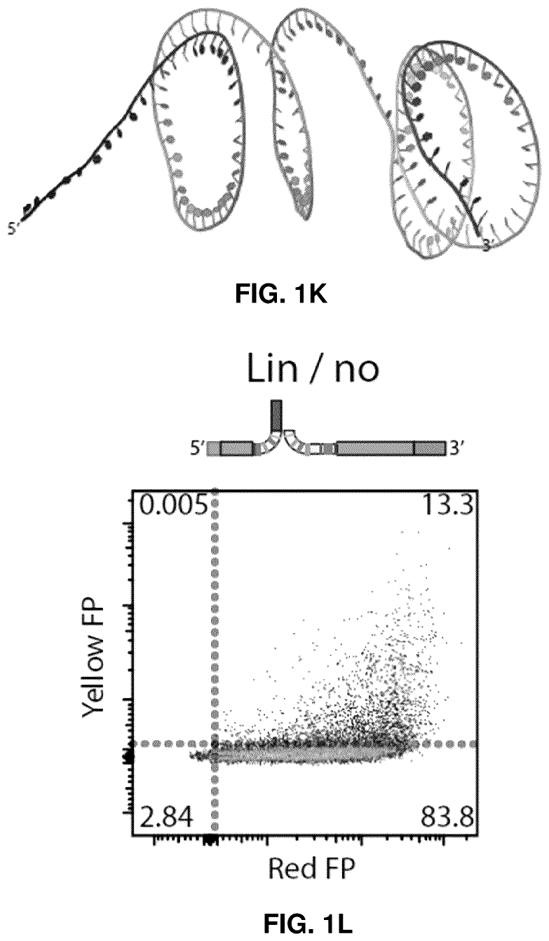

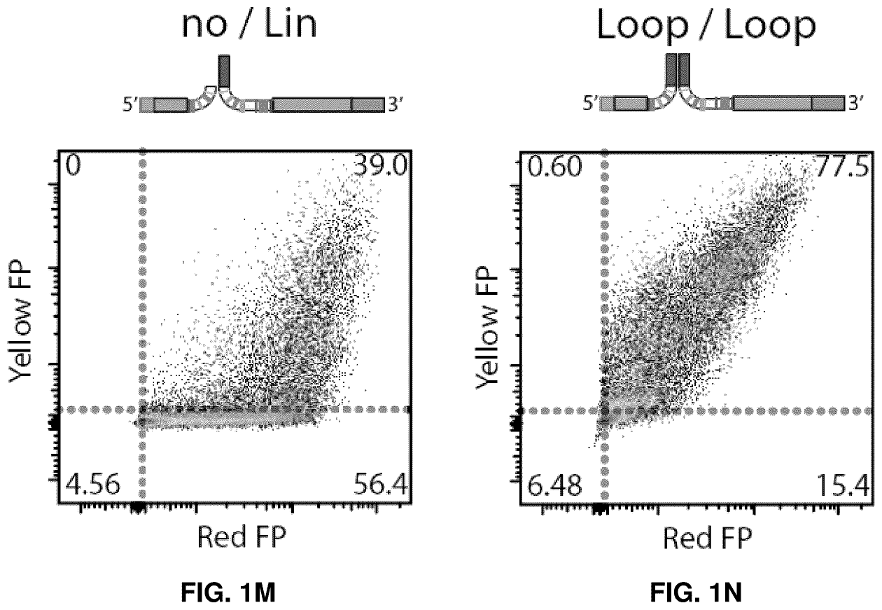

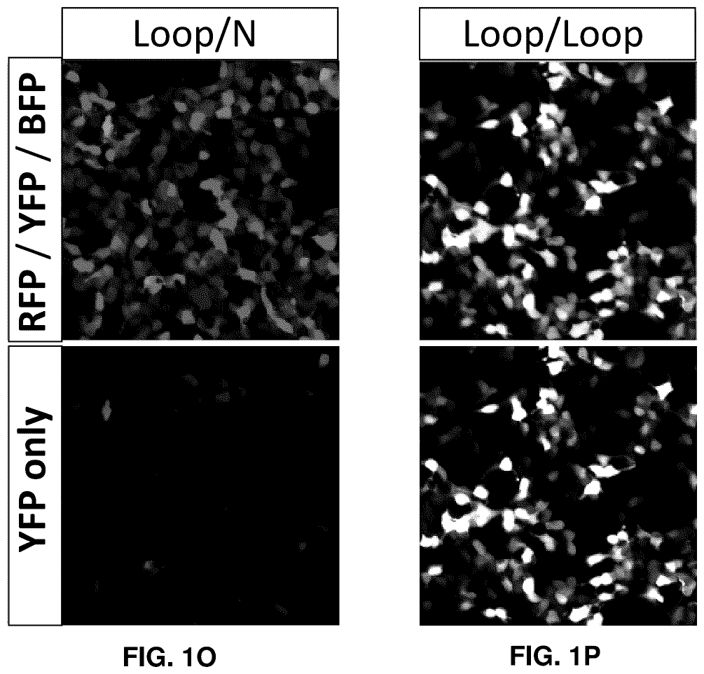

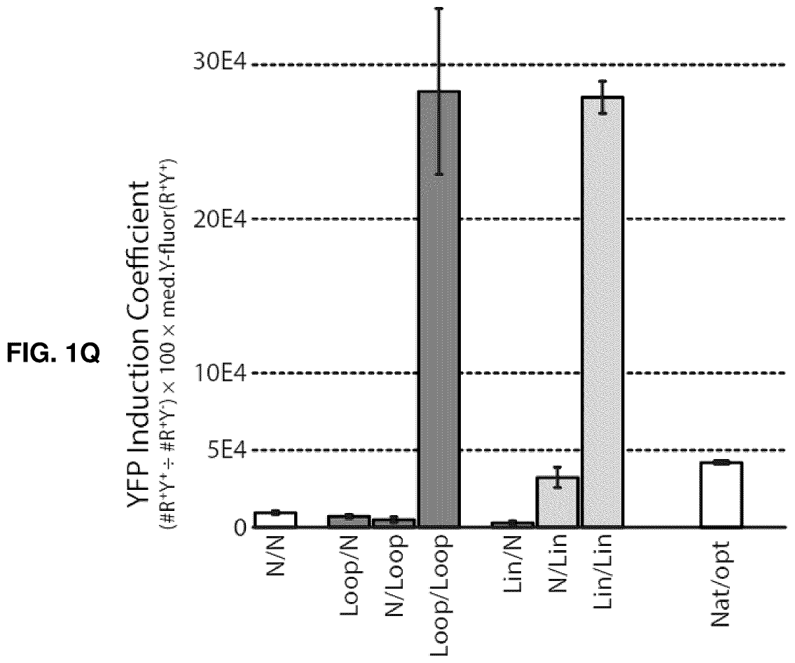

Molecule 110 of FIG. 6D includes the same features disclosed above for FIG. 1A, namely from 5′ to 3′, promoter 112, RNA encoding an N-terminal portion of a target protein 114 with a splice junction at its 3′-end, SD 116, optional DISE 118, optional ISE 120, dimerization domain 122, and optional polyadenylation sequence 124, but wherein first dimerization domain 122 has reverse complementary to third dimerization domain 204 of molecule 200.

Molecule 150 of FIG. 6D includes the same features disclosed above for FIG. 1A, namely from 5′ to 3′, promoter 152, second dimerization domain 154, optional ISE 156, branch point 158, polypyrimidine tract 160, SA 162, RNA encoding a C-terminal portion of a target protein 164 with a splice junction at its 5′-end, and optionally polyadenylation sequence 166, but wherein second dimerization domain 154 has reverse complementary to fourth dimerization domain 226 of molecule 200.

Molecule 200 allows for the joining of the N- and C-terminal coding RNAs 114, 164, by providing dimerization domains having reverse complementarity to dimerization domains 122, 154 of molecule 110 and molecule 150, respectively. Molecule 200 includes features from both molecule 110 and molecule 150, including two intronic sequences 230, 240. Specifically, molecule 220 includes from 5′ to 3′, promoter 210 (which can be the same or different than promoter 112 and/or 152), third dimerization domain 204 (which is the reverse complement to first dimerization domain 122 of molecule 110 in FIG. 6D), optional ISE 206, branch point 208, polypyrimidine tract 210, SA 212, RNA encoding a middle portion of a target protein 216 with a splice junction at both its 5′-end and 3′-end, SD 220, optional DISE 222, optional ISE 224, fourth dimerization domain 226 (which is the reverse complement to fourth dimerization domain 154 of molecule 150 in FIG. 6D), and optional polyadenylation sequence 228.

As shown in FIG. 6E, interaction and hybridization (base pairing) between first dimerization domain 122 of molecule 110 and third dimerization domain 204 of molecule 200, and interaction and hybridization (base pairing) between fourth dimerization domain 226 of molecule 200 and second dimerization domain 154 of molecule 150, allows the spliceosome components to recombine N-terminal coding sequence 114, middle coding sequence 216, and C-terminal coding sequence 164. Specifically the 3′ end of the N terminal protein coding sequence 114 is fused to the 5′ end of the middle protein sequence 216, and the 3′ end of middle protein sequence 216, is fused to the 5′ end of the C-terminal protein sequence 164 as a seamless junction between the three portions.

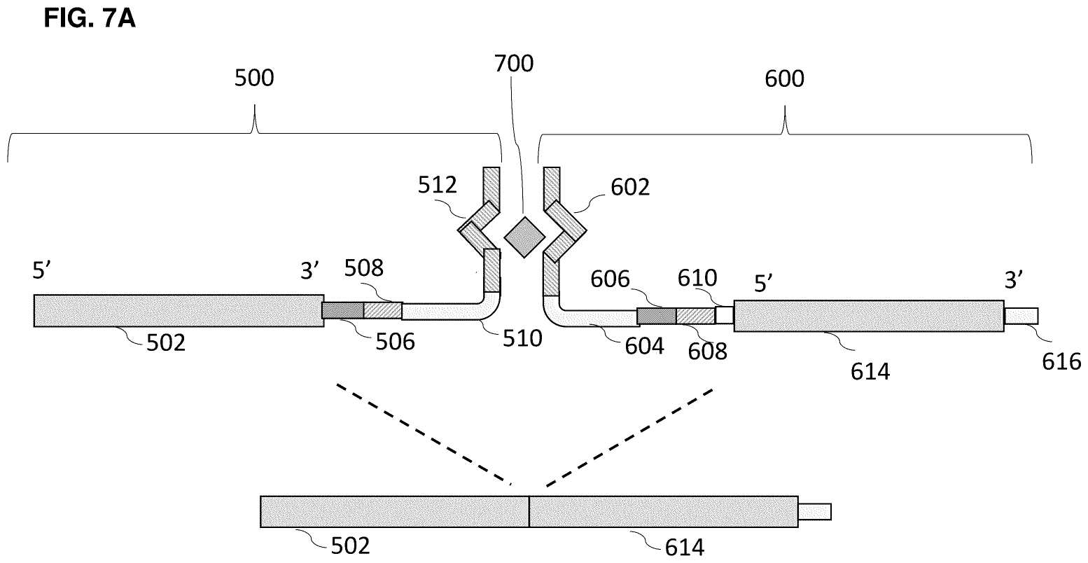

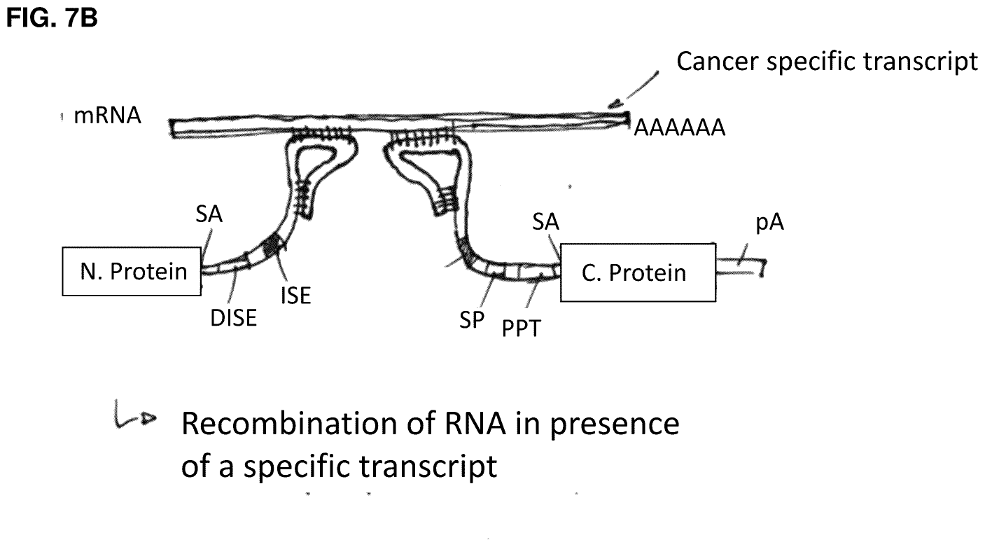

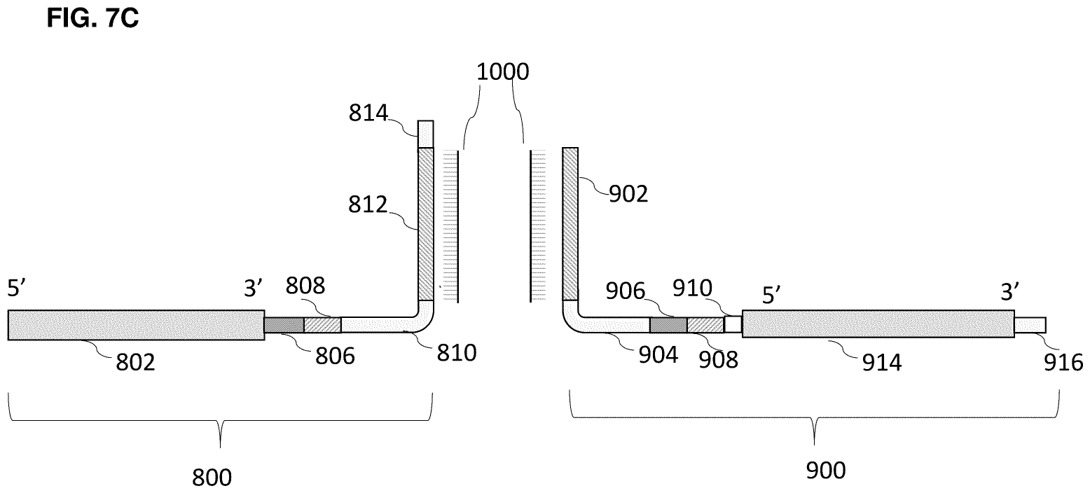

Alternative dimerization domains are shown in FIGS. 7A-7B and 9A. That is, as an alternative to using dimerization domains that hybridize to one another (e.g., 112 to 204, 226 to 154, FIGS. 6D, 6E), in one example aptamer sequences are used. As shown in FIG. 7A, in both synthetic nucleic acid molecules 500, 600, aptamer sequences 512, 602 are used instead of the dimerization domains, and the aptamers come together via their interaction with a target (such as adenosine, dopamine, or caffeine). In such an example, the aptamer sequence 512, 602 of each molecule 500, 600 can be the same, or even be different sequences. Molecule 500 of FIG. 7A includes the same features disclosed above for molecule 110 of FIG. 6A, namely from 5′ to 3′, promoter, RNA encoding an N-terminal portion of a target protein 502 with a splice junction at its 3′-end, SD 506, optional DISE 508, optional ISE 510, a first aptamer 512 instead of a first dimerization domain, and optional polyadenylation sequence. Similarly, molecule 600 of FIG. 7A includes the same features disclosed above for molecule 150 of FIG. 6A, namely from 5′ to 3′, promoter, aptamer 602 instead of second dimerization domain 154, optional ISE 604, branch point 606, polypyrimidine tract 608, SA 610, RNA encoding a C-terminal portion of a target protein 614 with a splice junction at its 5′-end, and optional polyadenylation sequence 616. Interaction of the two aptamers 512, 602, with each other or molecule 700 allows the spliceosome components to recombine N-terminal coding sequence 502 and C-terminal coding sequence 614. Specifically the 3′ end of the N terminal protein coding sequence 502 is fused to the 5′ end of the C terminal protein sequence 614 as a seamless junction between the two portions.

In some examples, aptamer sequences 512, 602 recognize (e.g., specifically bind) the same target 700 (FIG. 7A), or can even recognize different targets (wherein a synthetic molecule is also administered with the system provided herein, which includes each molecule specifically recognized by each aptamer, or the part of the molecule recognized by the aptamer, such as a caffeine/dopamine hybrid molecule). Exemplary targets recognized by aptamers include cellular proteins, small molecules, exogenous proteins, or an RNA molecule.

FIG. 7B shows an example similar to FIG. 7A. The dimerization domains (512, 602 FIG. 7A) recognize an RNA molecule. In the example shown in FIG. 7B, each domain recognizes a different portion of an mRNA molecule only expressed in target cells (cells where target protein expression is desired), such as a cancer-specific transcript. In such an example, the RNA coding sequences (502, 614 of FIG. 7A) only recombine in the presence of the specific RNA molecule recognized by the dimerization domains. Here, the target protein would only be expressed in cancer cells, not normal cells. Such a system allows for control of the target protein expression (e.g., a therapeutic protein for cancer, such as a toxin or a cytotoxic enzyme such as thymidine kinase with ganciclovir; thus in some examples the target protein is a toxin or thymidine kinase) in cancer cells, reducing undesirable side effects of expression the target protein in normal, non-cancer cells.

FIG. 7C provides an exemplary “off-switch” example. Here, the hybridization/binding of dimerization domains 812, 902 (which are reverse complements of one another) of synthetic nucleic acid molecules 800, 900 can be reduced by providing an anti-binding domain oligonucleotide (e.g, RNA or DNA) 1000 (which can be two different anti-binding domain oligonucleotides 1000, one that is the reverse complement of 812, and one that is the reverse complement of 912) that competes for the binding/hybridization. Anti-binding domain oligonucleotide 1000 can thus act as an “off-switch” for reconstitution of the protein encoded by N- and C-terminal coding portions 802 and 914, respectively. Molecule 800 of FIG. 7C includes the same features disclosed above for molecule 110 of FIG. 6A, namely from 5′ to 3′, promoter, RNA encoding an N-terminal portion of a target protein 802, splice junction 804, SD 806, optional DISE 808, optional ISE 810, dimerization domain 812, and optional polyadenylation sequence 814. Similarly, molecule 900 of FIG. 7B includes the same features disclosed above for molecule 150 of FIG. 6A, namely from 5′ to 3′, promoter, anti-dimerization domain 902, optional ISE 904, branch point 906, polypyrimidine tract 908, SA 910, RNA encoding a C-terminal portion of a target protein 914, and optional polyadenylation sequence 916. The two dimerization domains 812, 902 cannot interact/hybridize to each other in the presence of the anti-binding domain oligonucleotides 1000, and therefore prevents or reduces recombination of the N-terminal coding sequence 802 and C-terminal coding sequence 914. Such an application can be used to reduce or eliminate expression of the protein encoded by the system.

FIG. 9A provides an exemplary dimerization domain that uses kissing loop interactions instead of reverse complementary sequence hybridization for dimerization. Kissing loop interactions are formed when the bases in the loops of two RNA hairpins form interacting pairs between two RNA molecules.

Although FIGS. 6A-7C and 9A show embodiments where a system uses two synthetic nucleic acid molecules are used (i.e., the target protein coding sequence is split between two synthetic nucleic acid molecules), one skilled in the art will appreciate that such embodiments can be used similarly with more than two synthetic nucleic acid molecules, such as three, four, five, six, seven, eight, nine, or 10 synthetic nucleic acid molecules using the teachings herein.

In some examples, the system includes a nucleic acid molecule that suppresses expression of un-assembled/un-recombined fragments. In such an example, if the two or more portions of a full-length coding sequence (e.g., 114 of 110, 164 of 150 of FIG. 6A, respectively), did not recombine, the nucleic acid molecule would suppress expression of each portion of a full-length coding sequence that was not recombined into a full-length protein. For example, such a suppressive nucleic acid molecule can destabilize the RNA once outside the nucleus, prevent translation, stimulate translation from a shifted start codon, contain microRNA target sites, or contain protein degron or destabilization domains that when translated suppress the protein activity or flag it for degradation.

In one example, destabilization of the un-recombined RNA molecule is achieved by including a self-cleaving RNA sequence (e.g., Hammerhead ribozyme or HDV ribozyme) into the synthetic intron, for example at any position within intronic sequence 130 of FIG. 6A. In one example, cleaving the RNA molecule leads to a loss of the RNA stabilizing poly A tail, which can suppress expression of an un-recombined protein from open reading frame 114 of FIG. 6A. In one example, a self-cleaving RNA sequence is included at any position within s intronic sequence 170 of FIG. 6A to cleave off the 5′ terminal CAP which in one example can lead to reduced expression of an open reading frame that includes parts or the whole of coding sequence 164 of FIG. 6A. In one example self-cleaving RNA sequences are substituted with an RNA cleaving enzyme target site, such as a Csy4 target site.

In some examples, a suppressive nucleic acid molecule includes a start codon (ATG) or a Kozak enhanced start codon (GCCGCCACCATG (SEQ ID NO: 154) or GCCACCATG or ACCATG) at any position within intronic sequence 170 of FIG. 6A that directs translation of an open reading frame that is shifted −1,−2, +1, or +2 nucleotides relative to the open reading frame sequence 164 of FIG. 6A. In one example, un-assembled fragment expression is reduced or suppressed by using this decoy start codon strategy to direct translation away from the to be suppressed open reading frame of sequence 164 of FIG. 6A.

In some examples, a suppressive nucleic acid molecule includes one or more micro RNA target sites at any position within intronic sequence 130 of FIG. 6A, and/or at any position within intronic sequence 170 of FIG. 6A. If a particular RNA molecule (e.g., 110 or 150 in FIG. 6A) is exported from the nucleus, it becomes subject to micro RNA/small hairpin RNA dependent degradation which can suppress unintended un-joined fragment expression by degrading/suppressing un-joined RNA that was exported from the nucleus. In one example, such a micro RNA target sequence can be complementary to a micro RNA known to be expressed in the cell, or tissue, or animal into which the molecules 110 and 150 of FIG. 6A are introduced. In one example, this micro RNA target sequence is complementary to a sequence that is introduced into the cell, or tissue, or animal. In one example, such a microRNA can be expressed from an RNA-polymerase III dependent promoter in the form of a small hairpin RNA. In one example, such a microRNA can be expressed from an RNA polymerase II dependent promoter and embedded in a micro RNA processing loop (e.g., mir30 scaffold).

In some examples, destabilization of the un-recombined protein product from an open reading frame (e.g., 114 in FIG. 6) can be achieved by depleting stop codon occurrence in intronic sequence 130 of FIG. 6A and an additional inclusion of an RNA sequence coding for an in frame protein signal that can flag a protein for degradation (e.g., a degron sequence) that is placed at any position within intronic sequence 130 of FIG. 6A and which is in frame with the open reading frame that is extended out from sequence 114 of FIG. 6A. In one example a degron sequence can be that of a PEST sequence, or that of the CL1 degron sequence. Degron sequences used can employ proteasome-dependent, proteasome-independent, ubiquitin-dependent, or ubiquitin-independent pathways. In one example, un-recombined protein destabilization is enhanced by inclusion of several of the same or different degron sequences.

In some examples, destabilization of the un-recombined protein product from open reading frame sequence 164 in FIG. 6A is achieved by introduction of a start codon (ATG) followed by a degron sequence at any position within intronic sequence 170 in FIG. 6A which is in frame with an open reading frame within sequence 164 in FIG. 6. In this example, the degron sequence will be N-terminally joined to the un-recombined protein fragment that will be suppressed by being flagged for degradation.

### IV. Compositions and Kits

Compositions and kits are provided that include two or more of the synthetic nucleic acid molecules provided herein, wherein the synthetic nucleic acid molecule encode a full-length protein when recombined. In one example, the composition or kit includes two of the synthetic nucleic acid molecules provided herein, wherein each of the two synthetic nucleic acid molecules encodes a different portion of a target protein (i.e., N-terminal and C-terminal, wherein the whole coding sequence is generated when recombination between the two molecules occurs), such as one listed in Table 1 (or a therapeutic protein, such as a toxin or thymidine kinase). In one example, the composition or kit includes three of the synthetic nucleic acid molecules provided herein, wherein each of the three synthetic nucleic acid molecules encodes a different portion of a target protein (i.e., N-terminal, middle, and C-terminal, wherein the whole coding sequence is generated when recombination between the three molecules occurs), such as one listed in Table 1 (or a therapeutic protein, such as a toxin or thymidine kinase). In one example, the composition or kit includes four or more of the synthetic nucleic acid molecules provided herein, wherein each of the four of more synthetic nucleic acid molecules encodes a different portion of a target protein (i.e., N-terminal, first middle, second middle (and optionally additional middle), and C-terminal, wherein the whole coding sequence is generated when recombination between the four or more synthetic nucleic acid molecules occurs), such as one listed in Table 1 (or a therapeutic protein, such as a toxin or thymidine kinase). In one example, the composition or kit includes two or more sets of two or more of the synthetic nucleic acid molecules provided herein, wherein each set of synthetic nucleic acid molecules encodes a different target protein, such as two or more listed in Table 1 (and/or a therapeutic protein, such as a toxin or thymidine kinase).

In one example, each synthetic nucleic acid molecule in the composition or kit is part of a vector, such as AAV or other gene therapy vector. In one example, the composition or kit includes a cell, such as a bacterial cell or eukaryotic cell, that includes two or more disclosed synthetic nucleic acid molecule, wherein the synthetic nucleic acid molecules encode a full-length target protein when recombined.

Such compositions can include a pharmaceutically acceptable carrier (e.g., saline, water, glycerol, DMSO, or PBS). In some examples, the composition is a liquid, lyophilized powder, or cryopreserved.

In some examples, the kit includes a delivery system (e.g., liposome, a particle, an exosome, or a microvesicle) to direct cell type specific uptake/enhance endosomal escape/enable blood-brain barrier crossing etc.. In some examples, the kits further include cell culture or growth media, such as media appropriate for growing bacterial, plant, insect, or mammalian cells. In some examples, such parts of a kit are in separate containers. Exemplary containers include plastic or glass vials or tubes.

In some examples, each of two or more the synthetic nucleic acid molecules provided herein are in separate containers. In some examples, each of two or more sets of two or more of the synthetic nucleic acid molecules provided herein are in separate containers.

### V. Methods of Treatment

The disclosed methods and systems can be used to express any protein of interest, for example when a protein is too large to be expressed by a therapeutic virus (e.g., AAV) or when a complete gene sequence (e.g., endogenous promoter+coding sequence) is too large to be expressed by a therapeutic virus (e.g., AAV). In such cases, the coding sequence of the target protein may be divided into two or more portions and recombined in the correct order, allowing for the protein to be expressed when and where desired.

The subject to be treated can be any mammal, such as one with a monogenetic disorder, such as one listed in Table 1. In one example, the subject has cancer. Thus, humans, cats, pigs, rats, mice, cows, goats, and dogs, can be treated with the disclosed methods. In some examples, the subject is a human infant less than 6 months of age. In some examples, the subject is a human infant less than 1 year of age. In some examples, the subject is a human juvenile. In some examples, the subject is a human adult at least 18 years of age. In some examples, the subject is female. In some examples, the subject is male.

The two or more synthetic nucleic acid molecules provided herein used to treat a subject can be matched to the subject treated. Thus, for example, if the subject to be treated is a dog, a dog coding sequence for the target protein can be used and the intronic sequence can be optimized for expression in dog cells, and if the subject to be treated is a human, a human coding sequence for the target protein can be used and the intronic sequence can be optimized for expression in human cells.

The two or more synthetic nucleic acid molecules provided herein can be administered as part of a vector, such as an adeno-associated vector (AAV), for example AAV serotype rh.10. In some examples, vectors (e.g., AAV) including one of the two or more synthetic nucleic acid molecules provided herein are administered systemically, such as intravenously. Thus, if a coding sequence is divided between two synthetic nucleic acid molecules provided herein, two AAV's are administered, each AAV including one of the two synthetic nucleic acid molecules provided herein.

A therapeutically effective amount of two or more synthetic nucleic acid molecules provided herein is administered, for example in AAVs. In some examples, the two or more synthetic nucleic acid molecules provided herein when part of a viral vector (e.g., AAV) is administered at a dose of at least 1×10¹¹ genome copies (gc), at least 1×10¹² gc, at least 2×10¹² gc, at least 1×10¹³ gc, at least 2×10¹³ gc per subject, or at least 1×10¹⁴ gc per subject, such as 2×10¹¹ gc per subject, 2×10¹² gc per subject, 2×10¹³ gc per subject, or 2×10¹⁴ gc per subject. In some examples, the two or more synthetic nucleic acid molecules provided herein when part of a viral vector (e.g., AAV) is administered at a dose of at least 1×10¹¹ gc/kg, at least 5×10¹¹ gc/kg, at least 1×10¹² gc/kg, at least 5×10¹² gc/kg, at least 1×10¹³ gc/kg, or at least 4×10¹³ gc/kg, such as 4×10¹¹ gc/kg, 4×10¹² gc/kg, or 4×10¹³ gc/kg.

If adverse symptoms develop, such as AAV-capsid specific T cells in the blood, corticosteroids can be administered (e.g., see Nathwani et al., N Engl J Med. 365(25):2357-65, 2011).

Diseases that can be treated with the disclosed methods include any genetic disease of the blood (e.g. sickle cell disease, primary immunodeficiency diseases), HIV (such as HIV-1), and hematologic malignancies or cancers. Examples of primary immunodeficiency diseases and their corresponding mutations include those listed in Al-Herz et al. (Frontiers in Immunology, volume 5, article 162, Apr. 22, 2014, herein incorporated by reference in its entirety). Hematologic malignancies or cancers are those tumors that affect blood, bone marrow, and lymph nodes. Examples include leukemia (e.g., acute lymphoblastic leukemia, acute myelogenous leukemia, chronic lymphocytic leukemia, chronic myelogenous leukemia, acute monocytic leukemia), lymphoma (e.g., Hodgkin's lymphoma and non-Hodgkin's lymphoma), and myeloma. In some examples, the disease is a monogenetic disease. Table 1 provides a list of exemplary disorders and genes that can be targeted by the disclosed systems and methods. Additional examples are provided here rarediseases.info.nih.gov/diseases/diseases-by-category/5/congenital-and-genetic-diseases (list herein incorporated by reference). Any genetic disease caused by a lack of protein (e.g., recessive mutation) or an insufficiency of protein can benefit from the disclosed systems and methods. In cases where the coding region of the gene is relatively small, the disclosed systems and methods are useful to add regulatory sequences, such as tissue specific promoters or specific non-coding RNA segments, to direct gene expression to the appropriate cell types at the appropriate levels.

TABLE 1
Exemplary disorders and corresponding mutations

| Disease | Gene | Mutation |
|---|---|---|
| Blood cell disorder | | |
| sickle cell anemia | β-globin chain of | SNP (A to T) that gives rise to point |
| | hemoglobin | mutation (Glu−>Val at 6^{th} aa) |
| hemophilia | any of clotting factors I | |
| | through XIII | |
| hemophilia A | clotting factor VIII | large deletions, insertions, inversions, |
| | | and point mutations |
| hemophilia B | clotting factor IX | |
| Alpha-Thalassemia | HBA1 or HBA2 | Mutation or a deletion in |
| | | chromosome 16 p |
| Beta-Thalassemia | HBB | Mutations in chromosome 11 |
| Delta-Thalassemia | HBD | mutation |
| von Willebrand Disease | von Willebrand factor | mutations or deletion |
| pernicious anemia | MTHFR | |
| Fanconi anemia | FANCA, FANCC, | FANCA: c.3788_3790del |
| | FANCD2, FANCG, | (p.Phe1263del); |
| | FANCJ | c.1115_1118delTTGG (p.Val372fs); |
| | | Exon 12-17del; Exon 12-31del; |
| | | c.295C > T (p.Gln99X) |
| | | FANCC: c.711 + 4A > T (originally |
| | | reported as IVS4 + 4A > T); |
| | | c.67delG (originally reported as |
| | | 322delG) |
| | | FANCD2: c.1948 − 16T > G |
| | | FANCG; c.313G > T (p.Glu105X); |
| | | c.1077 − 2A > G; c.1480 + 1G > C; |
| | | c.307 + 1G > C; c.1794_1803del |
| | | (p.Trp599fs); c.637_643del |
| | | (p.Tyr213fs) |
| | | FANCJ: c.2392C > T (p.Arg798X) |
| Thrombocytopenic | ADAMTS13 | Missense and nonsense mutations |
| purpura | | |
| thrombophilia | Factor V Leiden | Mutation in the F5 gene |
| | Prothrombin | at position 1691 |
| | | Prothrombin G20210A |
| Primary | | |
| Immunodeficiency | | |
| Diseases | | |
| T-B+ SCID | IL-2RG, JAK3, defect in | |
| | gamma chain of receptors | |
| | for IL-2, -4, -7, -9, -15 and -21 | |
| T-B− SCID | RAG1, RAG2 | |
| WHIM syndrome | CXCR4 | heterozygous mutations (e.g., in the |
| | | carboxy-terminus); carboxy-terminus |
| | | truncation (e.g., 10-19 residues) |
| Other Primary | | |
| immune deficiency | | |
| (PID) syndromes | | |
| IL-7 receptor severe | IL7 receptor | |
| combined immune | | |
| deficiency (SCID) | | |
| Adenosine deaminase | ADA | |
| deficiency (ADA) | | |
| SCID | | |
| Purine nucleoside | PNP | |
| phosphorylase (PNP) | | |
| deficiency | | |
| Wiskott-Aldrich | WAS | More than 300 mutations identified |
| syndrome (WAS) | | |
| Chronic granulomatous | CYBA, CYBB, NCF1, | |
| disease (CGD) | NCF2, or NCF4 | |
| Leukocyte adhesion | Beta-2 integrin | |
| deficiency (LAD) | | |
| HIV | C-C chemokine receptor | Deletion of 32 bp in CCR5 |
| | type 5 (CCR5), | |
| | MSRB1 | |
| | HIV long terminal repeats | |
| | CSCR4 | |
| | P17 | |
| | PSIP1 | |
| Duchenne muscular | CCR5 | |
| dystrophy | DMD | |
| Glycogen storage | G6Pase | |
| disease type IA | | |
| Retinal Dystrophy | CEP290 | C2991 + 1655A > G |
| | ABCA4 | 5196 + 1216C > A; 5196 + 1056A > G; |
| | | 5196 + 1159G > A; 5196 + 1137G > A; |
| | | 938 − 619A > G; 4539 + 2064C > T |
| X-linked | MAGT1 | |
| immunodeficiency with | | |
| magnesium defect, | | |
| Epstein-Barr virus | | |
| infection, and neoplasia | | |
| (XMEN) | | |
| MonoGenetic | | |
| Disorders | | |
| Metachromatic | arylsulfatase | |
| leukodystrophy (MLD) | A (ARSA) | |
| Adrenoleukodystrophy | ABCD1 | |
| (ALD) | | |
| Mucopolysaccaridoses | IDS | |
| (MPS) disorders | IDUA | |
| Hunter syndrome | IDUA | |
| Hurler syndrome | SGSH, NAGLU, | |
| Scheie syndrome | HGSNAT, GNS | |
| Sanfilippo syndrome A, | GALNS | |
| B, C, and D | GLB1 | |
| Morquio syndrome A | ARSB | |
| Morquio syndrome B | GUSB | |
| Maroteaux-Lamy | HYAL1 | |
| syndrome | | |
| Sly syndrome | | |
| Natowicz syndrome | | |
| Alpha manosidosis | MAN2B1 | |
| Nieman Pick disease | SMPD1, NPC1, NPC2 | |
| types A, B, and C | | |
| Cystic fibrosis | cystic fibrosis | ΔF508 |
| | transmembrane | |
| | conductance regulator | |
| | (CFΓR) | |
| Polycystic kidney | PKD-1, PDK-2, PDK-3 | |
| disease | | |
| Tay Sachs Disease | HEXA | 1278insTATC |
| Gaucher disease | GBA | |
| Huntington's disease | HTT | CAG repeat |
| Neurofibromatosis | NF-1 and NF2 | CGA−>UGA−>Arg1306Term in NF1 |
| types 1 and 2 | | |
| Familial | APOB, LDLR, LDLRAP1, | |
| hypercholesterolemia | and PCSK9 | |
| Cancers | | |
| Chronic myeloid | BCR-ABL | fusion |
| leukemia (CML) | ASXL1 | |
| Acute myeloid | Chromosome 11q23 or | translocation |
| leukemia (AML) | t(9; 11) | |
| Osteosarcoma | RUNX2 | |
| Colorectal cancer | EPHA1 | |
| Gastric cancer, | PD-1 | |
| melanoma | | |
| Prostate cancer | Androgen receptor | |
| Cervical cancer | E6, E7 | |
| Glioblastoma | CD | |
| Neurological | | |
| disorders | | |
| Alzheimer's disease | NGF | |
| Metahchromatic | ARSA | |
| leukodystrophy | | |
| Multiple sclerosis | MBP | |
| Wiskott-Aldrich | WASP | |
| syndrome | | |
| X-linked | ABCD1 | |
| adrenoleukodystrophy | | |
| AACD deficiency | AADC | |
| Batten disease | CLN2 | |
| Canavan disease | ASPA | |
| Giant axonal | GAN | |
| neuropathy | | |
| Leber's hereditary optic | MT-ND4 | |
| neuropathy | | |
| MPS IIIA | SGSH, SUMF1 | |
| Parkinson's disease | GAD, NTRN, TH, AADC, | |
| | CH1, GDNF, AADC | |
| Pompe disease | GAA | |
| Spinal muscular | SMN | |
| atrophy type 1 | | |

Using the disclosed methods and systems can be used to treat any of the disorders listed in Table 1, or other known genetic disorder. The disclosed methods can also be used to treat other disorders, such as a cancer that can benefit from expression of a therapeutic protein in a cancer cell, such as a toxin or thymidine kinase. If the subject is administered two or more synthetic RNA molecules provided herein that express a full-length thymidine kinase, the subject is also administered ganciclovir. Treatment does not require 100% removal of all characteristics of the disorder, but can be a reduction in such. Although specific examples are provided below, based on this teaching one will understand that symptoms of other disorders can be similarly affected. For example, the disclosed methods can be used to increase expression of a protein that is not expressed or has reduced expression by the subject, or decrease expression of a protein that is undesirably expressed or has reduced expression by the subject. For example, the disclosed methods can be used to treat or reduce the undesirable effects of a genetic disease.

For example, the disclosed methods and systems can treat or reduce the undesirable effects of sickle cell disease by expressing a full-length wild-type ß-globin chain of hemoglobin. In one example the disclosed methods reduce the symptoms of sickle-cell disease in the recipient subject (such as one or more of, presence of sickle cells in the blood, pain, ischemia, necrosis, anemia, vaso-occlusive crisis, aplastic crisis, splenic sequestration crisis, and haemolytic crisis) for example a reduction of at least 10%, at least 20%, at least 50%, at least 70%, or at least 90% (as compared to no administration of the therapeutic nucleic acid molecule). In one example the disclosed methods decrease the number of sickle cells in the recipient subject, for example a decrease of at least 10%, at least 20%, at least 50%, at least 70%, at least 90%, or at least 95% (as compared to no administration of the therapeutic nucleic acid molecule).

For example, the disclosed methods and systems can treat or reduce the undesirable effects of thrombophilia by expressing a full-length wild-type factor V Leiden or prothrombin gene. In one example the disclosed methods reduce the symptoms of thrombophilia in the recipie7nt subject (such as one or more of, thrombosis, such as deep vein thrombosis, pulmonary embolism, venous thromboembolism, swelling, chest pain, palpitations) for example a reduction of at least 10%, at least 20%, at least 50%, at least 70%, or at least 90% (as compared to no administration of the therapeutic nucleic acid molecule). In one example the disclosed methods decrease the activity of coagulation factors in the recipient subject, for example a decrease of at least 10%, at least 20%, at least 50%, at least 70%, at least 90%, or at least 95% (as compared to no administration of the therapeutic nucleic acid molecule).

For example, the disclosed methods and systems can treat or reduce the undesirable effects of CD40 ligand deficiency by expressing a full-length wild-type CD40 ligand gene. In one example the disclosed methods reduce the symptoms of CD40 ligand deficiency in the recipient subject (such as one or more of, elevate serum IgM, low serum levels of other immunoglobulins, opportunistic infections, autoimmunity and malignancies) for example a reduction of at least 10%, at least 20%, at least 50%, at least 70%, or at least 90% (as compared to no administration of the therapeutic nucleic acid molecule s). In one example the disclosed methods increase the amount or activity of CD40 ligand deficiency in the recipient subject, for example an increase of at least 10%, at least 20%, at least 50%, at least 70%, at least 90%, at least 100%, at least 200% or at least 500% (as compared to no administration of the therapeutic nucleic acid molecule).

For example, the disclosed methods can be used to treat or reduce the undesirable effects of a primary immunodeficiency disease resulting from a genetic defect. For example, the disclosed methods and systems (which can use two or more synthetic RNA nucleic acid molecules to express a functional protein missing or defective in the subject, for example using AAV) can treat or reduce the undesirable effects of a primary immunodeficiency disease. In one example the disclosed methods reduce the symptoms of a primary immunodeficiency disease in the recipient subject (such as one or more of, a bacterial infection, fungal infection, viral infection, parasitic infection, lymph gland swelling, spleen enlargement, wounds, and weight loss) for example a reduction of at least 10%, at least 20%, at least 50%, at least 70%, or at least 90% (as compared to no administration of the therapeutic nucleic acid molecule). In one example the disclosed methods increase the number of immune cells (such as T cells, such as CD8 cells) in the recipient subject with a primary immune deficiency disorder, for example an increase of at least 10%, at least 20%, at least 50%, at least 70%, at least 90%, at least 95%, at least 100%, at least 200%, at least 300%, at least 400%, or at least 500% (as compared to no administration of the therapeutic nucleic acid molecule). In one example the disclosed methods reduce the number of infections ((such as bacterial, viral, fungal, or combinations thereof) in the recipient subject over a set period of time (such as over 1 year) with a primary immune deficiency disorder, for example a decrease of at least 10%, at least 20%, at least 50%, at least 70%, at least 90%, or at least 95%, (as compared to no administration of the therapeutic nucleic acid molecule).

For example, the disclosed methods can be used to treat or reduce the undesirable effects of a monogenetic disorder. For example, the disclosed methods (which can use two or more synthetic RNA nucleic acid molecules to express a functional protein missing or defective in the subject, for example using AAV) can treat or reduce the undesirable effects of a monogenetic disorder. In one example the disclosed methods reduce the symptoms of a monogenetic disorder in the recipient subject, for example a reduction of at least 10%, at least 20%, at least 50%, at least 70%, or at least 90% (as compared to no administration of the therapeutic nucleic acid molecule). In one example the disclosed methods increase the amount of normal protein not normally expressed by the recipient subject with a monogenetic disorder, for example an increase of at least 10%, at least 20%, at least 50%, at least 70%, at least 90%, at least 95%, at least 100%, at least 200%, at least 300%, at least 400%, or at least 500% (as compared to no administration of the therapeutic nucleic acid molecule).

For example, the disclosed methods can be used to treat or reduce the undesirable effects of a hematological malignancy in the recipient subject. In one example the disclosed methods reduce the number of abnormal white blood cells (such as B cells) in the recipient subject (such as a subject with leukemia), for example a reduction of at least 10%, at least 20%, at least 50%, at least 70%, or at least 90% (as compared to no administration of the disclosed therapies). In one example, administration of the disclosed therapies can be used to treat or reduce the undesirable effects of a lymphoma, such as reduce the size of the lymphoma, volume of the lymphoma, rate of growth of the lymphoma, metastasis of the lymphoma, for example a reduction of at least 10%, at least 20%, at least 50%, at least 70%, or at least 90% (as compared to no administration of the disclosed therapies). In one example, administration of disclosed therapies can be used to treat or reduce the undesirable effects of multiple myeloma, such as reduce the number of abnormal plasma cells in the recipient subject, for example a reduction of at least 10%, at least 20%, at least 50%, at least 70%, or at least 90% (as compared to no administration of the disclosed therapies).

For example, the disclosed methods can be used to treat or reduce the undesirable effects of a malignancy, such as one that results from a genetic defect in the recipient subject. In one example the disclosed methods reduce the number of cancer cells, the size of a tumor, the volume of a tumor, or the number of metastases, in the recipient subject (such as a subject with a cancer listed herein), for example a reduction of at least 10%, at least 20%, at least 50%, at least 70%, or at least 90% (as compared to no administration of the disclosed therapies). In one example, administration of the disclosed therapies can be used to treat or reduce the undesirable effects of a lymphoma, such as reduce the size of the tumor, volume of the tumor, rate of growth of the cancer, metastasis of the cancer, for example a reduction of at least 10%, at least 20%, at least 50%, at least 70%, or at least 90% (as compared to no administration of the disclosed therapies).

For example, the disclosed methods can be used to treat or reduce the undesirable effects of a neurological disease that results from a genetic defect in the recipient subject. In one example the disclosed methods increase neurological function in the recipient subject (such as a subject with a neurological disease listed above), for example an increase of at least 10%, at least 20%, at least 50%, at least 70%, at least 90%, at least 100%, at least 200%, at least 300%, at least 400%, or at least 500% (as compared to no administration of the disclosed therapies).

### Treatment of Duchenne Muscular Dystrophy (DMD)

Duchenne muscular dystrophy (DMD, MIM:310200) is a lethal hereditary disease characterized by progressive muscle weakness and degeneration. As the disease progresses, degenerating muscle fibres are replaced by fat and fibrotic tissue. DMD is rooted in deficiency of the gene dystrophin (MIM:300377). The dystrophin gene spans a region of 22 kbp, and is prone to mutations. Thus, DMD can in some cases sporadically manifest even in patients without a familial history of the disease-causing mutation. DMD is one of four conditions known as dystrophinopathies. The other three diseases that belong to this group are Becker Muscular dystrophy (BMD, a mild form of DMD); an intermediate clinical presentation between DMD and BMD; and DMD-associated dilated cardiomyopathy (heart-disease) with little or no clinical skeletal, or voluntary, muscle disease. Thus, in some examples a patient with DMD, BMD, an intermediate clinical presentation between DMD and BMD; or DMD-associated dilated cardiomyopathy (heart-disease) with little or no clinical skeletal, or voluntary, muscle disease, is treated with the disclosed systems and methods.

The disclosed methods and systems can be used to treat the monogenic cause of DMD, that is expression of dystrophin. Dystrophin has a long coding region, such as dystrophin. Current methods of expressing dystrophin from a single AAV utilize shortened/truncated versions of dystrophin (micro-dystrophin and mini-dystrophin). Several of these truncated dystrophin delivery therapies are being tested in Phase I/II clinical trials (NCT03362502, NCT00428935, NCT03368742, NCT03375164). Although these truncated versions of dystrophin may ameliorate the worst consequences of dystrophin deficiency in DMD, they are not expected to have full functionality when compared to full-length dystrophin as the truncated versions are missing key domains in the rod and hinge region of the full-length protein. The disclosed methods and systems alleviate the size restriction of the transgenic payload of AAV by using “multiplexed” AAV combinations, because multiple AAV viruses can efficiently infect the same cell when introduced at high multiplicity of infection (MOI, i.e., high titer).

Thus, in some examples, a composition that includes two or more AAVs, each containing one of a set of disclosed synthetic RNA molecules, is administered (e.g., i.v.) to a DMD subject in a therapeutically effective amount, such as a set that includes two, three, four or five different synthetic RNA molecules (each in a different AAV), which when recombined, result in a full-length dystrophin coding sequence.

### EXAMPLE 1

### Synthetic RNA Dimerization and Recombination Domains

FIG. 1A depicts a schematic of exemplary vector designs. The protein coding sequence of a yellow fluorescent protein (YFP) is split into an N-terminal and a C-terminal fragment. The N-terminal fragment is appended with a synthetic intronic sequence that contains a consensus splice donor sequence (SD), a downstream intronic splice enhancer sequence (DISE), two intronic splice enhancer sequences (ISE), and a stable stem loop BoxB element (boxB). This splicing optimized intronic sequence is followed by a binding domain as described in panels FIGS. 1E-IN. The C-terminal fragment of YFP is preceded by the complementary binding domain sequence, a stable stem loop BoxB element (boxB), three intronic splice enhancer sequences (ISE), a consensus branch point sequence (BP), a polypyrimidine tract (PPT) and a splice acceptor consensus sequence (SA). For transfection control, the N-terminal fragment is coexpressed with a red fluorescent protein from a bidirectional promoter and the C-terminal fragment is coexpressed with a blue fluorescent protein. Once expressed the two RNA molecules, termed 5′ trspRNA and 3′trspRNA will dimerize and get recombined through a process called RNA recombination.

FIG. 1B depicts transfection of only the N-terminal expression plasmid does not lead to YFP fluorescence. Flow cytometry displaying 20 k RFP+cells.

FIG. 1C depicts transfection of only the C-terminal expression plasmid does not lead to YFP fluorescence. Flow cytometry displaying 20 k BFP+cells.

FIG. 1D depicts expression of N-terminal and C-terminal fragments without binding domains shows low levels of YFP induction. Flow cytometry displaying red and green fluorescence values for 20 k BFP+cells.

FIG. 1E depicts rationally designed dimerization/binding domain in a looped configuration. Segments of hypodiverse exclusively pyrimidine or exclusively purine containing sequences are interspaced with stable stem sequences. RNA folding predictions shows 6 stretches of open sequence available for base pairing between the binding domain and its complementary sequence.

FIG. 1F depicts 3D rendering of the “looped” dimerization domain configuration.

FIG. 1G depicts negative control with no binding domain on the C-terminal half. Flow cytometry displaying red and green fluorescence values for 20 k BFP+cells.

FIG. 1H depicts negative control with no binding domain on the N-terminal half. Flow cytometry displaying red and green fluorescence values for 20 k BFP+cells.

FIG. 1I depicts matching binding domains on both N- and C-terminal half shows strong YFP induction in 90% of the cells. Flow cytometry displaying red and green fluorescence values for 20 k BFP+cells.

FIGS. 1J-1N depict data equivalent to that in FIGS. 1E-1I for a configuration of a binding domain with a stretch of 150 hypodiverse exclusively pyrimidine or exclusively purine containing sequence resulting in a fully open configuration.

FIG. 1O depicts representative fluorescence images for cells shown in FIG. 1G.

FIG. 1P depicts representative fluorescence images for cells shown in FIG. 1L.

FIG. 1Q depicts a comparison of conditions shown in FIG. 1D, FIGS. 1G-1I, and FIGS. 1L-1N. YFP induction coefficient is calculated: (#R+Y+÷#R+Y−)×100×med. Y-fluor(R+Y+). For comparison the recombination efficiency of a native intron (intron I of the mouse parvalbumin gene) on the N-terminus and an optimized binding domain for that intron on the C-terminal fragment are shown (white bar). This illustrates the benefits of the optimized synthetic RNA dimerization and recombination domains.

### EXAMPLE 2

### Reconstitution of Protein from Three Synthetic Fragments

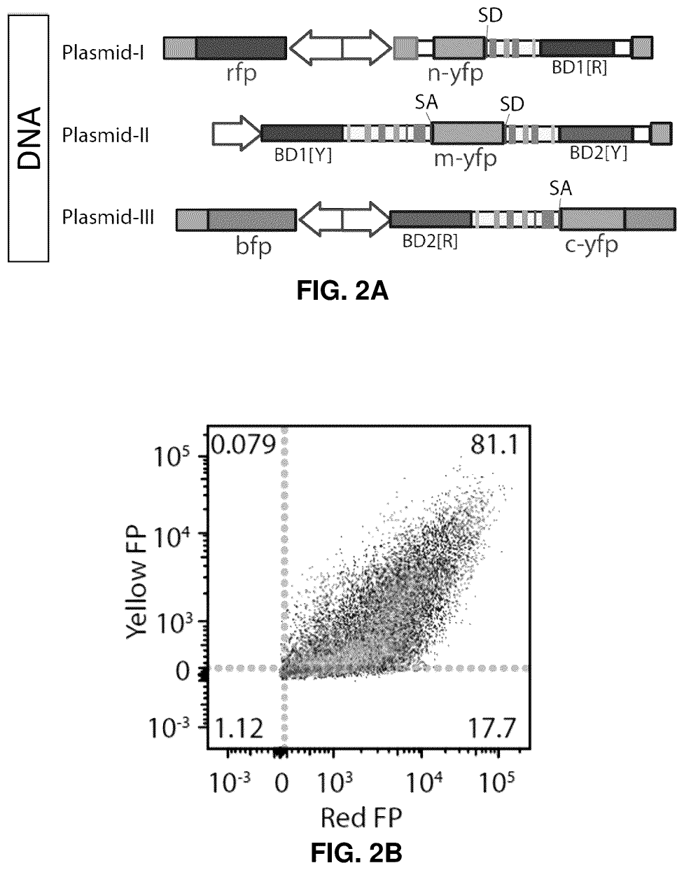

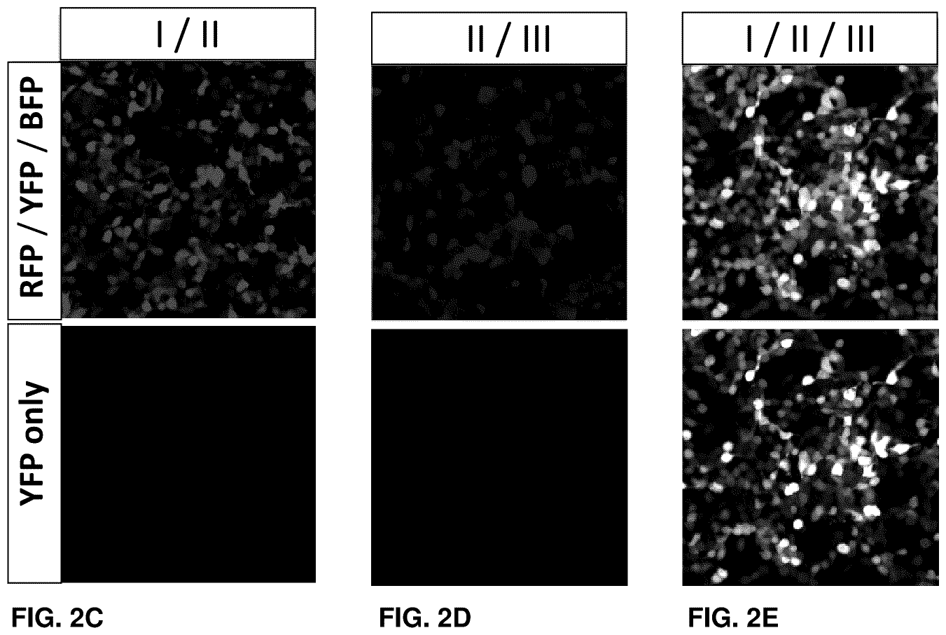

FIG. 2A depicts an exemplary schematic of vector designs. The protein coding sequence of a YFP is split into an N-terminal fragment, a middle fragment (m-yfp) and a C-terminal fragment. The junction of the n and m fragments is joined by a looped design binding domain (BD1) and the junction between m and c fragments is joined by a looped binding domain (BD2). The pyrimidine (Y) and purine (R) sequences are arranged to avoid self-circularization of the m-fragment and avoid direct recombination of the N- and C-fragment. The N-terminal fragment is co-expressed with red fluorescent protein as a transfection control, the C-terminal fragment is coexpressed with blue fluorescent protein as a transfection control.

FIG. 2B depicts matching binding domains on all three fragments shows strong YFP induction in 80% of the cells. Flow cytometry displaying red and green fluorescence values for 20 k BFP+cells.

FIG. 2C depicts representative fluorescent image of expression of the n and m fragment only shows no YFP fluorescence (negative control).

FIG. 2D depicts representative fluorescent image of expression of the m and c fragment only shows no YFP fluorescence (negative control).

FIG. 2E depicts representative fluorescent image showing that strong YFP fluorescence is induced by co-transfection of all three fragments.

### EXAMPLE 3

### In Vivo Delivery of Reconstituted Full-Length YFP Divided into Two Portions

Reconstitution of a YFP coding sequence from two fragments is achieved by using two synthetic RNA sequences, wherein one included the n-terminal coding half fragment of YFP, and one included the c-terminal coding half fragment (FIG. 3A) (SEQ ID NOS 1 and 2). Each fragment was expressed from AAV2/8 after systemic (iv) administration in newborn (P3) mouse pups. A total of 1.88E11 viral genomes for each of the two fragments were administered per mouse. Expression of YFP was detected 3 weeks later in the liver, heart muscle, and skeletal muscle using fluorescence microscopy.

As shown in FIG. 3B, expression of full-length YFP was detected in the liver of the juvenile mouse, while uninjected liver showed no YFP expression.

As shown in FIG. 3C, expression of full-length YFP was detected in the heart muscle of the juvenile mouse, while uninjected heart muscle showed no YFP expression.

As shown in FIG. 3D, expression of full-length YFP was detected in the skeletal muscles of the leg, while uninjected liver showed no YFP expression.

Thus, the disclosed systems can be used to express full-length proteins in vivo, from two or more separate synthetic RNA molecules.

### EXAMPLE 4

### In Vivo Delivery of Reconstituted Full-Length YFP Divided into Three Portions

Reconstitution of a YFP coding sequence from three fragments is achieved by using three synthetic RNA sequences, wherein one included the n-terminal fragment of YFP, one included a middle fragment of YFP, and one included the c-terminal fragment (FIG. 4A) (SEQ ID NOS: 145, 146 and 2 respectively).

Each fragment was expressed from AAV2/8 after intramuscular injection into the e tibialis anterior muscle of newborn (P3) mouse pups. A total of 1E11 viral genomes for each of the fragments was administered intramuscularly. Expression of YFP was detected 3 weeks later in the skeletal muscle using fluorescence microscopy.

As shown in FIG. 4B, expression of full-length YFP fluorescence was observed in the tibialis anterior muscle.

Thus, the disclosed systems can be used to express full-length proteins in vivo, from three or more separate synthetic RNA molecules.

### EXAMPLE 5

### In Vivo Delivery of Reconstituted Full-Length Protein

To demonstrate the feasibility of a three-part sRdR system in vivo, a combination of either two or three AAV-transfer plasmids (the DNA precursor plasmids of AAV) containing fragments of the YFP were transcutaneously electroporated into the tibialis anterior (TA) hindlimb muscle of adult mice. Efficient reconstitution of both the two part split-YFP system as well as the three part split-YFP system was observed five days after intramuscular electroporation (FIGS. 5A-5F).

FIGS. 5A-5F depict efficient reconstitution of YFP from two and from three fragments in adult mouse tibialis anterior muscle. FIG. 5A depicts N-terminal and C-terminal halves of YFP coding sequences are equipped with synthetic RNA-dimerization and recombination domains. FIG. 5B depicts two AAV transfer plasmids expressing these two fragments were electroporated transcutaneously into adult mouse tibialis anterior (TA) muscle and strong fluorescence was detected at 5 days post electroporation. FIG. 5C shows no fluorescence was detectable in contralateral non-injected TA. FIG. 5D depicts N-terminal, middle, and C-terminal YFP coding sequence are equipped with synthetic RNA-dimerization and recombination domains linking each fragment to its adjacent fragment(s). FIG. 5E depicts transcutaneous electroporation of three AAV transfer plasmids expressing these three fragments. Strong YFP fluorescence is detected indicating efficient reconstitution of YFP from three fragments. FIG. 5F depicts fluorescence in contralateral non-injected TA. Fluorescent channel is overlaid onto grey scale photographs for context.

Data are also provided on pages 13-14 of Exhibit A, where two or three vectors were used to express YFP in liver, cardiac muscle and skeletal muscle (two AAV vectors), and in skeletal muscle (three AAV vectors).

Hence the synthetic RNA-dimerization and recombination system provided herein can be deployed in the muscle. Based on these results, one can substitute the YFP coding sequence with a dystrophin (or other gene) coding sequence to achieve therapeutic full-length dystrophin (or other gene) expression from AAVs into a desired subject and/or tissue.

### EXAMPLE 6

### Delivery of Reconstituted Full-Length Dystrophin to Treat DMD

An effective gene therapy using full-length dystrophin for patients who suffer from Duchenne muscular dystrophy (DMD) has remained challenging, because the coding sequence of this large protein exceeds the capacity of most viral vectors. Adeno-associated viruses (AAVs) are a common and the preferred method of gene delivery in gene replacement therapy. AAVs are non-toxic, well tolerated, and lead to long term expression of the replacement gene without random integration into the genome. However, the dystrophin gene is too large to be delivered by a single virus. If broken down into fragments, full-length dystrophin can only be delivered using a minimum of three viruses. Smaller versions of dystrophin called “micro-Dystrophin” or “mini-Dystrophin” are currently being tested for dystrophin gene replacement therapy, but these truncated versions of dystrophin are not expected to have full functionality as they are missing key domains in the rod and hinge section of the protein. To date, past attempts to overcome this limitation have not yielded the efficiency required for treating DMD.

Provided herein is a novel RNA based technology that can be used to efficiently reconstitute the coding sequence of large genes, including dystrophin, from multiple serial fragments. Using this technology in combination with AAV as a delivery vector, full-length dystrophin will be expressed in a murine model (as well as pig and canine models) for DMD. In one example the subject is a human adult, juvenile, or infant with DMD. For example, the disclosed methods and systems can be used to deliver synthetic RNA-dimerization and recombination domains encoding full-length dystrophin over two or three AAVs (e.g., each AAV delivering a half or a third of the full-length coding sequence). In one example, the AAVs are myotropic AAVs (e.g., those that preferentially infect muscles). This approach can be used to ameliorate or prevent the onset of dystrophy symptoms in a mouse or canine model for DMD, as well as human subjects.

Part 1: Construct efficiently reconstituted three-way split dystrophin expression cassettes. Three expression cassettes are constructed that efficiently reconstitute the full-length dystrophin coding sequence in vitro while each individual expression cassette is within the packaging limit of conventional AAV vectors. To achieve therapeutically effective levels of dystrophin, the expression system can be optimized to achieve roughly physiological levels of dystrophin or moderately supraphysiological levels. Up to 50-fold overexpression of dystrophin is tolerated without adverse effects. The dystrophin coding sequence can be split at a number of different points along its length. Efficiency of reconstitution, however, is affected by the local RNA microenvironment and maximization of reconstitution efficiency is done empirically by comparing efficiency of several possible split points. The natural dystrophin coding sequence can be codon optimized for optimal expression and modified to accommodate maximal reconstitution efficiency. It is expected that the full-length dystrophin coding sequence can be reconstituted from a three-way split precursor using the synthetic RNA-dimerization and recombination approach herein disclosed. In screening different configurations, the set of three expression cassettes that lead to the most efficient reconstitution of dystrophin (e.g., approximately physiological or moderately supraphysiological levels) are selected. Experiments can be performed in HEK293T or Human Skeletal Muscle Cells (HSKMC, either primary or trans-differentiated). Using endogenous vs. exogenous specific quantitative RT-PCR probes, and by epitope tag detection in the exogenous dystrophin protein and Western blot analysis, reconstitution efficiencies will be determined different configurations of the split/reconstituted dystrophin.

Part 2: Maximize full-length dystrophin expression over non-reconstituted fragments. Suppression of fragmented background expression of non-reconstituted dystrophin can be achieved by modification of the synthetic RNA-dimerization and recombination domains. Non-reconstituted fragment expression caused by inefficiencies in RNA-recombination may lead to background expression of dystrophin fragments. Further, suppression of this fragmented background expression may be achieved by modification of the synthetic RNA-dimerization and recombination domains. With the disclosed approach, each fragment of dystrophin is transcribed separately. Reconstitution occurs on the RNA level. Each individual fragment can therefore potentially be translated without being reconstituted. In a western blot, with full-length dystrophin running at roughly 430 kDa, these fragments would run at sizes of about ⅔ (˜290 kDa) and ⅓ (˜140 kDa) of that. The synthetic RNA-dimerization and recombination domains can be optimized to avoid non-reconstituted fragment expression and favor full length expression of dystrophin. This can for example be achieved by strategically placing degron sequences, disrupting RNA nuclear export of non-recombined fragments, and introducing decoy translation initiation points. Experiments are carried out in HEK293T and HSKMC. The dystrophin coding sequence can be bookended with epitope tags that allow for identification and quantification of not fully reconstituted fragments of dystrophin using western blot analysis. Cellular distribution of these dystrophin fragments will be assessed using immunohistochemistry in skeletal human muscle cells. Additionally, quantitative assessment of fragment suppression will be done using conventional molecular biology techniques, including quantitative RT PCR across the recombination junctions will be used to determine how efficient the reconstitution on an RNA level occurs. It is expected that low levels of fragmented dystrophin expression will be observed. By modifying the synthetic RNA-dimerization and recombination domains, these fragments can be suppressed.

Part 3. Create high-titer AAV stocks of full-length dystrophin modules for in vitro and in vivo expression. Dystrophin expressing AAVs will be produced with high purity and viral genome counts higher than 3E13 GC/ml. Three myotropic AAV serotypes will be produced: AAV2/8, AAV2/9, and AAV2/rh10. A tripartite split fluorescent protein, a tripartite split of a full-length dystrophin bookended with epitope tags (see Part 2 above), and a non-tagged tripartite split of full-length dystrophin will be produced, resulting in 27 high-titer AAV preparations. Systemic delivery of therapeutic AAV particles requires high concentration large virus preparations. To achieve reconstituted expression of dystrophin form three separate viruses, repeated administration of the virus may be performed. AAV production in HEK293T cells. Iodixanol or CsCl purification. All batches will be tested in vitro in HEK293T and human skeletal muscle cells. As outlined in Part 1 and 2, reconstitution efficiency and unwanted fragment expression will be assessed.

Part 4. Measure expression/reconstitution levels of FLD-AAV modules in vivo and tissue distribution in vivo of full-length dystrophin expressing AAV modules. The same are assessed for a tripartite split fluorescent protein, as surrogate indicator. For in vivo delivery, direct intramuscular (cardiac and skeletal muscles) and systemic intravenous delivery in newborn and juvenile mice will be compared. Direct muscle injection of FLD-AAV may result in efficient expression of full-length dystrophin as indicated in the Examples above. Systemic delivery of FLD-AAV will be examined using immunohistochemistry and western blot analysis. Different routes of administration, including direct intramuscular and systemic intravenous delivery, in newborn and juvenile mice will be compared. The analysis will focus on: (1) skeletal muscles (major forelimb, hindlimb, shoulder, abdominal and, face muscles) and differential infectivity of fast vs. slow twitch muscles, will be assessed by comparing tibialis anterior and soleus muscles, (2) cardiac muscle expression, and (3) liver expression. This cohort of animals will be monitored for possible adverse effects of the high-titer AAV injections.

Although direct muscular injection of AAVs represents an approach to delivering the FLD-AAV modules (which in light of the results in FIGS. 5A-5F is likely to be successful), it is nonetheless desirable from a clinical perspective to achieve full-length dystrophin expression using systemic i.v. delivery of the virus. In vitro FLD-AAV testing will be used to determine how AAV copy number and reconstituted dystrophin levels correlate. Tissue distribution and efficiency of reconstitution will be assessed in vivo, and different delivery paradigms (e.g., serotype, viral titer, route of application, number of repeat applications) will be examined to achieve optimal tissue distribution. Tissue coverage and expression levels will be assessed. Beneficial outcomes can be achieved even if only a portion of muscle fibers express dystrophin (e.g., normal heart function with only about 50% of cardiomyocytes being dystrophin deficient under non-stress conditions). Both, physiological and supraphysiological levels of dystrophin are of therapeutic value. Quantitative assessment will be performed as outlined in Part 1 & 2. In vivo intramuscular and systemic virus application will be performed in neonatal or juvenile mice under aseptic condition.

Part 5. Treat DMD mouse model (mdx) with FLD-AAV and assess disease onset/progression. FLD-AAV delivery in neonatal mdx mice may prevent the onset and progression of myopathy and cardiomyopathy. After optimization of the viral delivery of reconstituted full-length dystrophin (Parts 1-4) FLD-AAV treatment will be administered to a mouse model of DMD. These mice, depending on the genetic background they are bred, present with myopathy that is notably less pronounced than human DMD. Mice with the genetic background that presents with a more severe phenotype (D2.B10-Dmdmdx) show increased hind-limb weakness, lower muscle weight, fewer myofibers, and increased fat and fibrosis. These parameters can be compared between wild-type controls, treated mdx, and untreated mdx mice. The desired outcome is an amelioration or prevention of disease onset/progression.

Two mouse lines, C57BL/10ScSn-Dmdmdx/J, and D2.B10-Dmdmdx/J, which carry a mutation in the dystrophin gene are used. FLD-AAV is delivered according to parameters established as described under Part 4. Animals are injected in the first postnatal week, in a time window before onset of myonecrosis in mdx mice. Wild-type, treated-mdx and vehicle/sham-treated-mdx mice are e assessed for behavioral and anatomical signs of skeletal and cardiac myopathy. Using kinematic and electromyographic testing equipment, performance of these mice in a variety of motor tasks is assessed, such as balance beam, grip strength, horizontal ladder, treadmill speed challenge, over ground locomotor kinematic assessment, and swimming kinematic assessment (ambient temperature and cold water challenge). It will be determined whether FLD-AAV therapy can prevent the presentation of cardiomyopathy in mdx mice following chemical challenge.

The desired outcome of these experiments would be an amelioration or prevention of disease onset/progression.

### EXAMPLE 7

### Delivery of Reconstituted Full-Length MYO7A treat Usher Syndrome

A first half of the MYO7A coding sequence is appended with a synthetic RNA dimerization and recombination domain and expressed from a first vector/plasmid. The second half of MYO7A is appended to the complementary RNA dimerization and recombination domain and expressed from a second vector/plasmid. If expressed together in the same cell the two halves of MYO7A are recombined to form the full-length MYO7A transcript which is then translated into protein.

### EXAMPLE 8

### Transcriptional/Expressional Logic Gate

Breaking a target gene into two nonfunctional halves that get expressed from either two different promoters or using two different delivery vehicles can result in an intersectional expression pattern.

For example, promoter 1 of a first synthetic nucleic acid molecule provided herein can drive expression of the N-terminal half of the coding sequence in for example cell types A, B, and C, while promoter 2 of a second synthetic nucleic acid molecule provided herein drives expression of the C-terminal half in a subset of cells A, D, E, and F. In such an example, the effector gene encoding the target protein is only expressed in the overlapping area (in this example in cell population A).

A similar intersectionality can be used by making the two halves conditionally expressed, for example, under the condition of the presence of a recombinase. Another level at which intersectionality can be achieved is by delivering the two halves with two viruses that have different tropisms.

### EXAMPLE 9

### Complementation

The disclosed methods and systems can be used to make any gene (and corresponding target protein) into complementation parts (similar to the principle of alpha complementation of LacZ), by encoding two non-functional halves on separate plasmids that only become active when both plasmids are present.

### EXAMPLE 10

### Trigger RNA

The disclosed systems and methods can be configured such that reconstitution of the two or more portions of the RNA coding sequences of the target protein depends on the presence of a specific “trigger” RNA molecule. As shown in FIG. 7B, in this example, the dimerization domains of each synthetic nucleic acid molecule are not reverse complements of one another, but instead specifically hybridize to adjacent regions of a third RNA molecule, a “trigger RNA”, which serves as a bridge to bring two synthetic nucleic acid molecules together. In this example, the system can “report” the presence of a specific RNA molecule which allows for “cell type specific triggering” of a reporter/effector protein.

### EXAMPLE 11

### Inclusion of Stabilizing Element in 3′-UTR

This example describes methods used to evaluate recombination of split coding sequences in the presence of a sequence in the 3′-UTR that stabilizes RNA. Woodchuck hepatitis posttranscriptional regulatory element 3 (WPRE3) was used as an exemplary stabilizing sequence. One skilled in the art will appreciate that other RNA sequence stabilizers can be used in place of WPRE3.

Median YFP fluorescence was measured by flow-cytometry for a two-way split YFP that is reconstituted using the disclosed synthetic RNA dimerization and recombination approach. The C-terminal YFP coding fragment is followed by a poly adenylation signal only (w/o WPRE3) or by a truncated version of the woodchuck hepatitis posttranscriptional regulatory element, WPRE3followed by a poly adenylation signal (labelled w/WPRE3). The N-terminal YFP coding fragment is coexpressed with a red fluorescent protein from a bidirectional promoter for transfection control. The C-terminal fragment is co-expressed with a blue fluorescent protein from a bidirectional promoter as transfection control. Cells with equal red and blue fluorescent control values between conditions are compared.

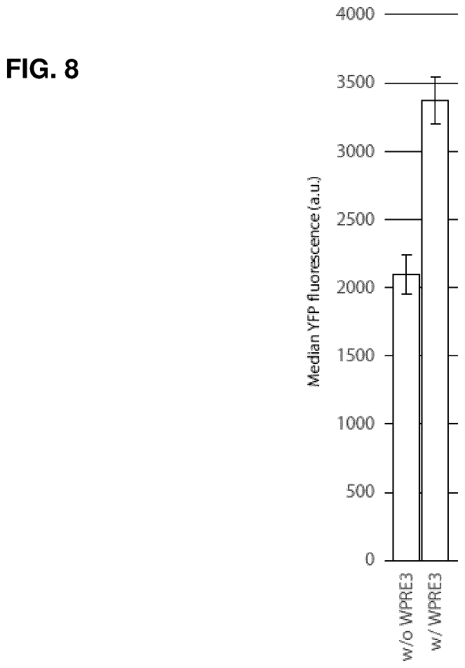

As shown in FIG. 8, inclusion of a stabilizing element in the 3′-UTR increased expression efficiency of the recombined full-length YFP by about 50-60%. This enhancement is observed even though WPRE sequences stimulate nuclear export of the RNA molecule they are contained in, which may have negatively impacted the RNA joining reaction (and thus gene expression) by shuttling molecule 150 of FIG. 6A outside the nucleus before the spliceosome mediated RNA joining can occur and thus rendering it non-functional.

Thus, the disclosed synthetic RNA molecules (such as any of SEQ ID NOS: 1, 2, 3, 4, 5, 6, 7, 8, 9, 10, 11, 12, 13, 14, 15, 16, 17, 18, 19, 20, 21, 22, 23, 24, 25, 145, 146, 17, and 148) can be modified to further include a RNA sequence stabilizer.

### EXAMPLE 12

### Effect of Binding Domain Length on Reconstitution Efficiency

Binding domain length was assessed as follows. YFP was split into two non-fluorescent halves (SEQ ID NOS: 1 and 2, but with different length binding domains). Reconstitution efficiency for different length binding domains (ranging from 50 to 500 nucleotides) was assessed in cultured HEK 293t cells. N-terminal YFP is expressed from a bidirectional CMV promoter with a Red Fluorescent Protein (RFP) as a transfection control. C-terminal YFP is expressed from a bidirectional CMV promoter with a Blue Fluorescent Protein (BFP) as a transfection control. For the different binding domain lengths, YFP median fluorescence intensity was compared. Cells with matching RFP and BFP transfection levels are compared between conditions.

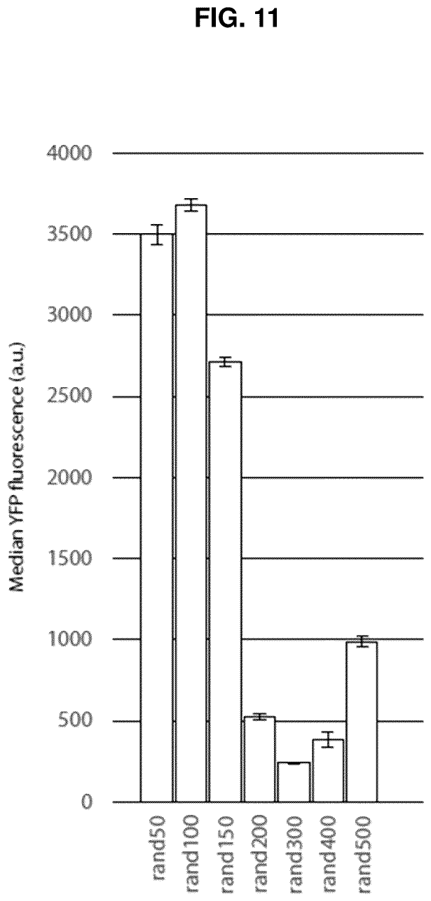

As shown in FIG. 11, all of the molecules achieved some level of expression of the full-length YFP, with varying degrees of reconstitution efficiency. Although maximal performance was observed with binding domain lengths of 150 bp and below (e.g. 50-150 bp), binding domain lengths of up to 500 bp were still able to recombine and express full-length YFP.

### EXAMPLE 13

### Effect of Splicing Enhancer Sequences

This example describes methods used to assess the effect of including one or more intronic splicing enhancer sequences (e.g., 118, 120, 156 in FIG. 6A) in the disclosed synthetic introns.

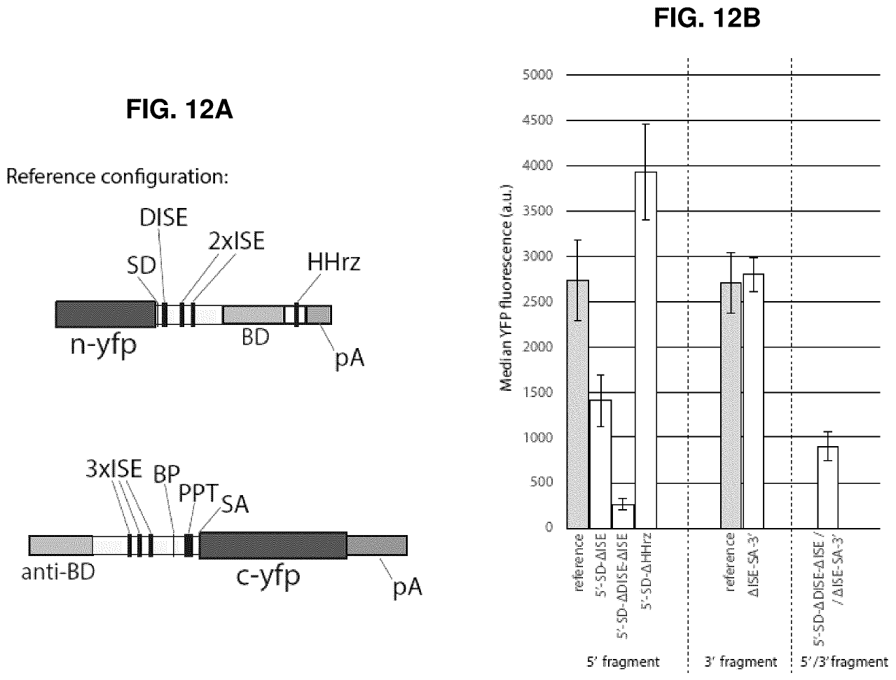

YFP was split into two non-fluorescent halves (FIG. 12A). Reconstitution efficiency for different intron configurations was assessed in cultured HEK 293t cells. N-terminal YFP was expressed from a bidirectional CMV promoter with a Red Fluorescent Protein (RFP) as a transfection control. C-terminal YFP was expressed from a bidirectional CMV promoter with a Blue Fluorescent Protein (BFP) as a transfection control. For the different intron configurations, YFP median fluorescence intensity is compared. Cells with matching RFP and BFP transfection levels are compared between conditions.

As shown in FIG. 12A, the 5′ molecule (SEQ ID NO: 1) includes the coding region of the N-terminal portion of YFP (n-yfp), followed by a splice donor sequence (SD), a downstream intronic splicing enhancer (DISE), and two intronic splicing enhancers (2xISE), a binding domain (BD), a self-cleaving hammerhead ribozyme (HHrz), ending with a poly adenylation signal (pA). The 3′ molecule (SEQ ID NO: 2) includes the complementary binding domain (anti-BD), followed by three intronic splicing enhancer sequences (3xISE), a branch point (BP), a polypyrimidine tract (PPT), a splice acceptor sequence (SA), the c-terminal proton of the YFP coding sequence, ending with a poly adenylation signal (pA).

As shown in FIG. 12B, inclusion of splice enhancers to both the 5′ and the 3′ molecules increases reconstitution efficiency of the full-length YFP. Removal of the splice enhancers reduces the reconstitution efficiency of the two coding sequences by about 50-90%. In the first column, YFP is reconstituted using the reference configuration (SEQ ID NOS: 1 and 2), the second column shows the reconstitution efficiency with deletion of the ISE elements in the 5′ fragment, the third column shows reconstitution efficiency after deletion of the ISE and the DISE in the 5′ fragment. The fourth column shows the reconstitution efficiency after deletion of the HHrz in the 5′ fragment. The fifth column shows reconstitution efficiency using the reference configuration. The sixth column shows reconstitution efficiency after deletion of the ISE elements in the 3′ fragment. The seventh shows reconstitution efficiency after deletion of the ISE in both 5′ and 3′ fragment and the DISE in the 5′ fragment.

### EXAMPLE 14

### Dual Projection Tracing

This example describes methods used to perform dual projection tracing by reconstitution of full-length flp recombinase (Flpo) from two fragments (SEQ ID NOS: 147 and 148). As shown in FIG. 13A, Flp recombinase is split into two non-functional haves. The N-terminal half of Flpo is appended with a synthetic intron and dimerization domain (RNA end joining module, REJ). The C-terminal half of Flpo is prepended with a synthetic intron and a binding domain (REJ-module). Upon infection of a cell of both constructs, the full length Flpo recombinase mRNA and subsequently the functional recombinase protein are produced by reconstitution of the two fragments. FIG. 13B shows a schematic of an flp activity reporter mouse carrying a flpo dependent red fluorescent protein (tdTomato) (Rosa-CAG-frt-STOP-frt-tdTomato). The two halves of flpo are packaged into separate adeno-associated viruses (retrogradely transported serotype AAV2/retro). The AAV2/retro-n-flpo is injected in the left primary motor cortex of the mouse, the AAV2/retro-c-flpo is injected in the right primary motor cortex of the mouse.

As shown in FIGS. 13C-13D, cells with dual projections to both primary motor cortices are labelled in red. Hoechst staining (nuclei) is shown for context.

### EXAMPLE 15

### Expression of Long Protein In Vivo

This example describes methods used to achieve efficient expression of oversized cargo in cell culture and in vivo in the mouse primary motor cortex.

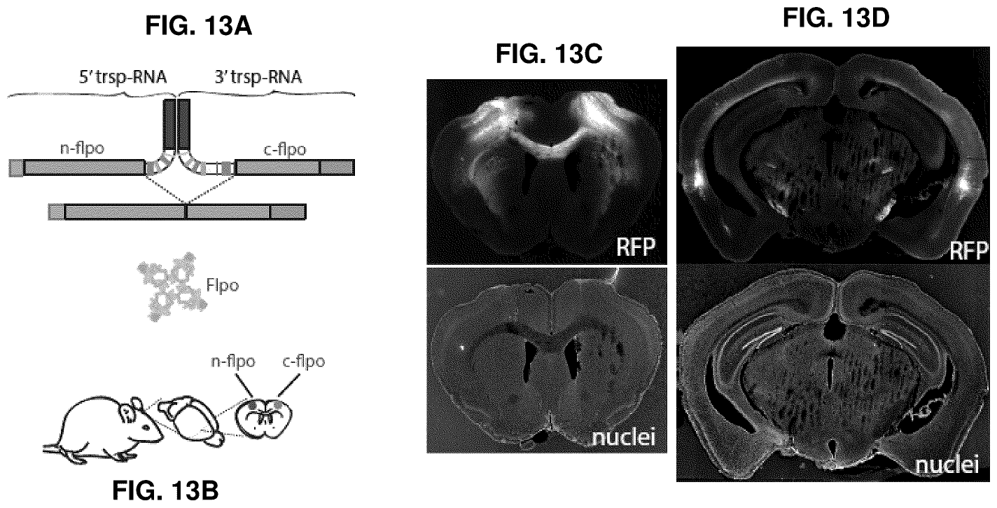

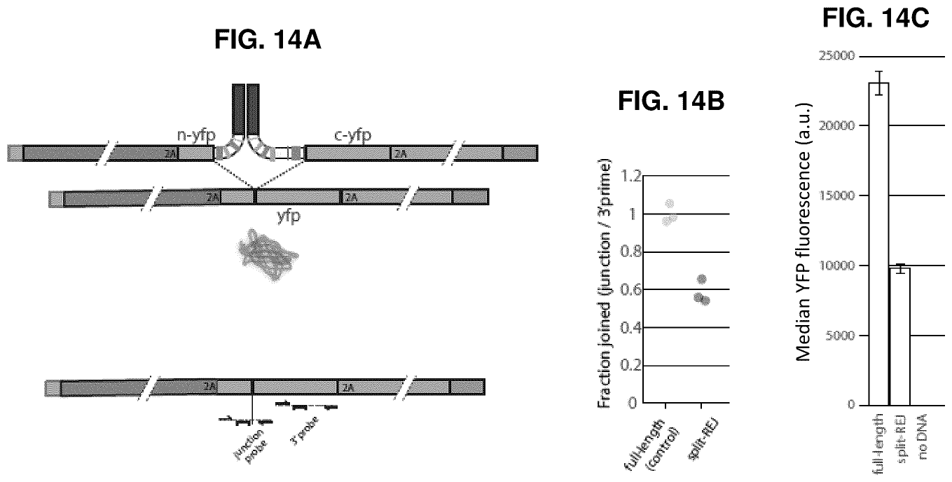

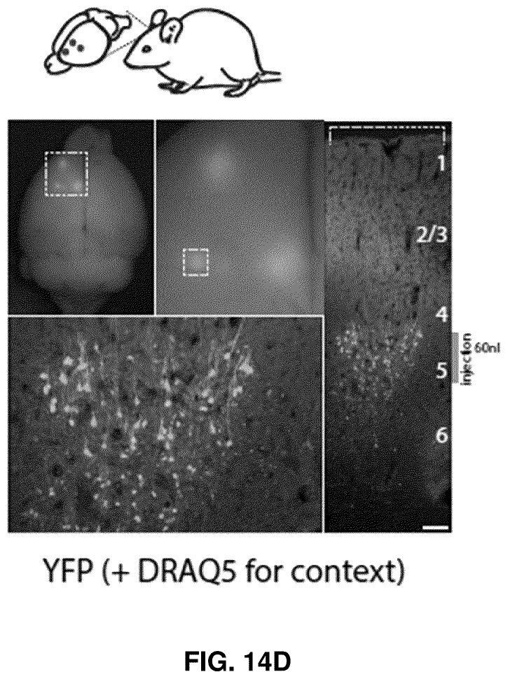

To simulate a large disease-causing gene that fills up the adeno-associated virus (AAV) cargo capacity of two viruses (i.e., it exceeds single AAV packaging capacity), a split YFP was embedded inside a large uninterrupted open reading frame. N-terminally (i.e. on the 5′ side) the YFP is flanked with long stuffer sequences (i.e. an uninterrupted open reading frame) followed by a 2A self-cleaving peptide sequence. On the C-terminus (i.e., 3′ side) the YFP coding sequence is followed by a 2A self-cleaving peptide sequence and then followed by a long stuffer sequence (i.e., and uninterrupted open reading frame) (FIG. 14A). The resulting RNA molecules expressed are each about 4000 nt between the transcriptional start site and the poly adenylation site. The N-terminal (5′ fragment; SEQ ID NO: 22) contains a stuffer open reading frame which is followed by a self-cleaving 2A sequence, followed by the N-terminal portion of YFP, followed by a synthetic intron and a dimerization domain (kissing loop architecture). The C-terminal (3′ fragment; SEQ ID NO: 23) is composed of a complementary binding domain, a synthetic intron sequence, followed by the C-terminal portion of YFP, followed by a self-cleaving 2A sequence, followed by a stuffer open reading frame, followed by a poly adenylation signal. During translation, the 2A sequences flanking the YFP result in the cleaving off of the N and C-terminal stuffer sequences and the production of functional YFP protein.

To determine reconstitution efficiency on an RNA level, two probe based (5′-hydrolysis) quantitative real-time PCR assays are used. The first assay spans a sequence fully contained in the 3′ exonic YFP sequence (labelled 3′ probe). The second assay spans the junction between the 5′ and the 3′ exonic YFP sequence (labelled junction probe). Reconstitution efficiency is calculated as the ratio of (junction probe count)/(3′ probe count).

Quantitative real-time PCR analysis of reconstitution efficiency of the oversize YFP constructs in HEK 293t cells was performed. Full-length oversized YFP is used as reference. The full-length oversized YFP ratio is set to 1 (FIG. 14B). Ratio of reconstituted is expressed as fraction of full-length (labelled split-REJ (split RNA end joining)). Reconstitution efficiency is calculated as follows: junction/3′prime. As shown in FIG. 14B about 60% of the RNAs joined in the split-REJ system.

Reconstituted YFP protein expression from full-length oversized YFP expression and split-REJ expression is assessed by flow cytometry of transiently transfected HEK 293t cells. As shown in FIG. 14C, the split REJ system achieved about a 45% joining efficiency, even with the large cargo.

in vivo analysis of reconstitution of the large YFP protein was performed as follows. 60 nl of adeno-associated virus 2/8, containing 3E9 vg/injection/fragment, was injected into the primary motor cortex of the mouse. Tissue was harvested 10 days post injection. As shown in FIG. 14D, YFP fluorescence is readily detectable in the bulk tissue (top left, top middle panel, macroscopic top view of the mouse brain, YFP fluorescence plus auto-fluorescence for context are shown). Strong YFP signal is detected at and around the virus injection site in layer 5 of the motor cortex (right panel, cortical layers are numbered 1 to 6, approximate injection depth is indicated by gray bar, scale bar =100 micrometers). Thus, the disclosed system can be used to express large proteins in vivo.

### EXAMPLE 16

### Expression of Factor VIII

This example describes methods used to achieve efficient reconstitution of full-length human coagulation factor VIII (FVIII).

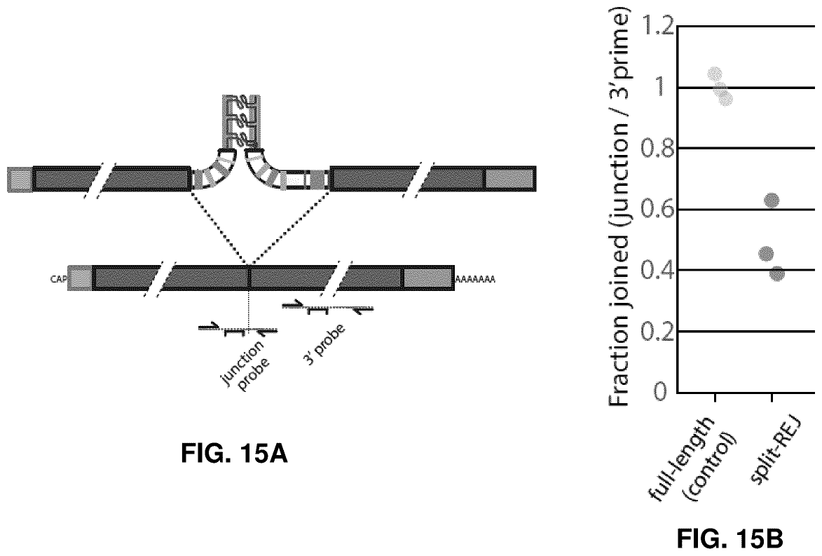

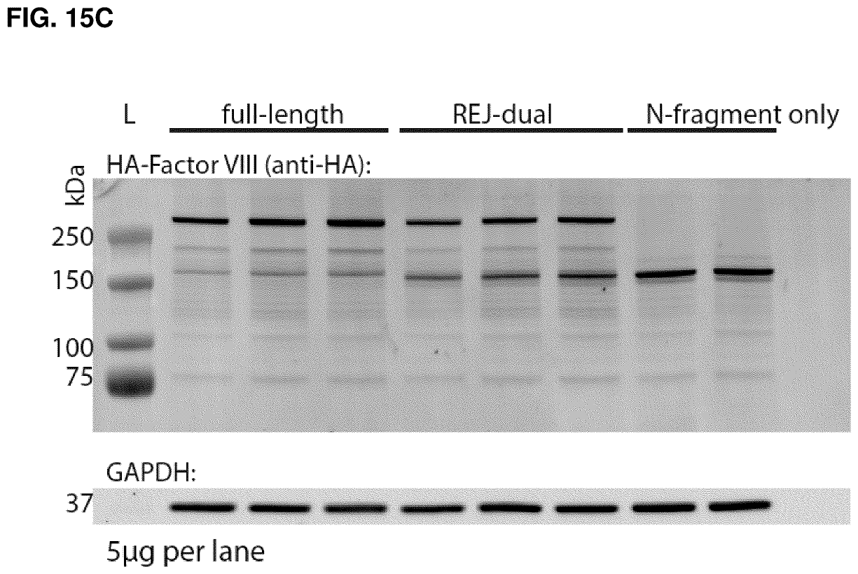

A schematic of the 5′ and 3′ molecules used are shown in FIG. 15A (SEQ ID NOS: 24 and 25, respectively). Each half includes about 3.8 kb of FVIII coding sequence. The 5′-sequence containing the N-terminal half (e.g., 110 of FIG. 6A) of FVIII is followed by an efficient synthetic intron and a binding domain. The 3′-sequence containing the C-terminal half (e.g., 150 of FIG. 6A) is preceded by the complementary binding domain and an efficient synthetic intron sequence. To determine reconstitution efficiency on an RNA level, two probe based (5′-hydrolysis) quantitative real-time PCR assays are used. The first assay spans a sequence fully contained in the 3′ exonic FVIII sequence (labelled 3′ probe). The second assay spans the junction between the 5′ and the 3′ exonic FVIII sequence (labelled junction probe). Reconstitution efficiency is calculated as the ratio of (junction probe count)/(3′ probe count).

PCR quantification of reconstitution efficiency after two days of expression in HEK 293t cells was performed. Full-length FVIII is used as reference. Full-length FVIII ratio is set to one. Reconstituted FVIII assay ratios are expressed as fraction of full-length (labelled split-REJ). As shown in FIG. 15B, a reconstitution efficiency of about 40-60% was achieved (that is about 40-60% of the two RNAs joined in the split-REJ system).

To demonstrate expression of FVIII in vitro, Western blotting was used. FVIII was tagged with an HA-tag at the N-terminus. Constructs are expressed in HEK 293t cells for 2 days. As shown in FIG. 15C, the disclosed split-REJ system successfully expressed full-length FVIII in vitro.

Based on these observations, expression of a full-length FVIII protein in vivo can be achieved, for example to treat hemophilia A. For example, a first half of a FVIII coding sequence is appended with a synthetic RNA dimerization and recombination domain and expressed from a first vector/plasmid. The second half of FVIII is appended to the complementary RNA dimerization and recombination domain and expressed from a second vector/plasmid. If expressed together in the same cell the two halves of FVIII are recombined to form the full-length FVIII transcript which is then translated into protein. For example, a sequence having at least 80%, at least 90%, at least 95%, at least 96%, at least 97%, at least 98%, at least 99% or 100% sequence identity to SEQ ID NO: 24, which includes an N-terminal FVIII coding sequence, and SEQ ID NO: 25 which includes a C-terminal FVIII coding sequence, can be utilized for in vivo expression.

### EXAMPLE 17

### Expression of Abca4

This example describes methods used to achieve efficient reconstitution of full-length human ATP binding cassette subfamily A member 4 (Abca4).

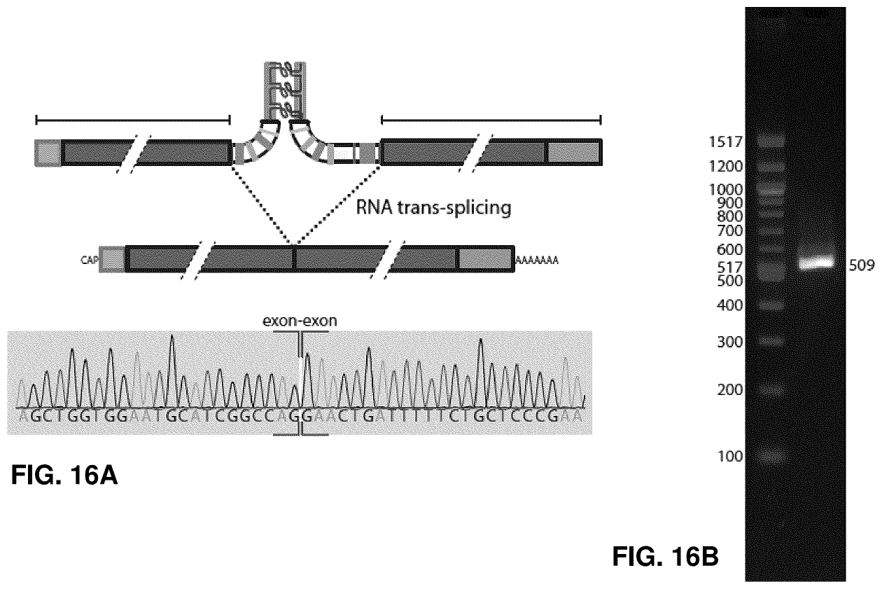

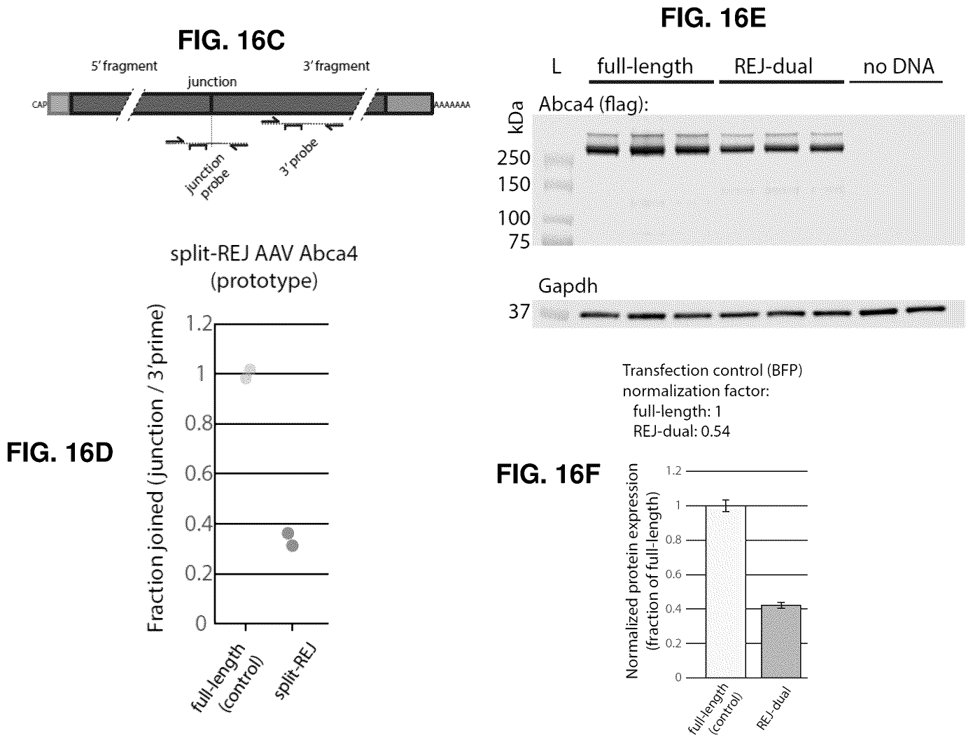

A schematic of the 5′ and 3′ molecules used are shown in FIG. 16A (SEQ ID NOS: 19 and 21, respectively). The 5′ half includes about 3.6 kb of Abca4 coding sequence, the 3′ half about 3.2 kb of the Abca4 coding region plus a C-terminal 3×FLAG tag. The 3′-sequence containing the C-terminal half (e.g., 150 of FIG. 6A) is preceded by the complementary binding domain and an efficient synthetic intron sequence. A Sanger sequencing trace across the junction is shown.

As shown in FIG. 16B, PCR amplification of the junction demonstrates faithful joining of the two coding sequences. To determine reconstitution efficiency on an RNA level, two probe based (5′-hydrolysis) quantitative real-time PCR assays are used (FIG. 16C). The first assay spans a sequence fully contained in the 3′ exonic Abca4 sequence (labelled 3′ probe). The second assay spans the junction between the 5′ and the 3′ exonic Abca4 sequence (labelled junction probe). Reconstitution efficiency is calculated as the ratio of (junction probe count)/(3′ probe count). PCR quantification of reconstitution efficiency after two days of expression in HEK 293t cells is shown in FIG. 16D. Full-length Abca4 is used as reference. Average full-length Abca4 ratio is set to one. Reconstituted Abca4 assay ratios are expressed as fraction of full-length (labelled split-REJ). As shown in FIG. 16D, a reconstitution efficiency of about 35% was achieved (that is about 30-40% of the two RNAs joined in the split-REJ system).

To demonstrate expression of Abca4 in vitro, Western blotting was used. Abca4 is tagged with a 3×FLAG-tag at the C-terminus. Constructs are expressed in HEK 293t cells for 2 days. As shown in FIG. 16E, the disclosed split-REJ system successfully expressed full-length Abca4 in vitro.

Quantification of the western blot is shown in FIG. 16F. To normalize for differential transfection efficiency between conditions, the full-length plasmid and the C-terminal plasmid co-express a Blue Fluorescent Protein for transfection control. BFP concentration in each sample was determined by dot blot and used to normalize between conditions. As shown in FIG. 16F reconstituted Abca4 is expressed at approximately 40% of the levels when compared with direct full-length expression. Hence, the protein levels as determined by western blot, track well with the RNA reconstitution efficiency determined by qPCR.

Based on these observations, expression of a full-length ABCA4 protein in vivo can be achieved, for example to treat Stargardt's Disease. For example, a first half of the ABCA4 coding sequence is appended with a synthetic RNA dimerization and recombination domain and expressed from a first vector/plasmid. The second half of ABCA4 is appended to the complementary RNA dimerization and recombination domain and expressed from a second vector/plasmid. If expressed together in the same cell the two halves of ABCA4 are recombined to form the full-length ABCA4 transcript which is then translated into protein. For example, a sequence having at least 80%, at least 90%, at least 95%, at least 96%, at least 97%, at least 98%, at least 99% or 100% sequence identity to SEQ ID NO: 20 (FIGS. 10R-10U), which includes an N-terminal Abca4 coding sequence, and SEQ ID NO: 21 (FIGS. 10V-10Z) which includes a C-terminal Abca4 coding sequence, can be utilized for in vivo expression.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

We claim:

1. A system for expressing a target protein, comprising:
(a) a first synthetic nucleic acid molecule, comprising from 5′ to 3′,
a first promoter;
an RNA molecule encoding an N-terminal portion of the target protein operably linked to the first promoter, which includes a splice junction at a 3′-end of the RNA molecule encoding the N-terminal portion of the target protein;
a splice donor; and
a first dimerization domain; and
(b) a second synthetic nucleic acid molecule; comprising from 5′ to 3′,
a second promoter;
a second dimerization domain operably linked to the second promoter, and having reverse complementarity to the first dimerization domain;
a branch point sequence;
a polypyrimidine tract;
a splice acceptor; and
an RNA molecule encoding a C-terminal portion of a target protein, which includes a splice junction at a 5′-end of the RNA molecule encoding the C-terminal portion of a target protein.


SEQUENCE LISTING (154 sequences)

SEQ ID NO: 1 - DNA, 1491 bp, synthetic construct
  misc_feature 1..1491: note = Synthetic nucleic acid sequence
  source 1..1491: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 2 - DNA, 1302 bp, synthetic construct
  misc_feature 1..1302: note = Synthetic nucleic acid sequence
  source 1..1302: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaacaactcgtggaggagagaaaagagacgagttgtgaagaagagagaggaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaggacctttgagaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagt

SEQ ID NO: 3 - DNA, 404 bp, synthetic construct
  misc_feature 1..404: note = Synthetic nucleic acid sequence
  source 1..404: mol_type = other DNA
gtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtacc

SEQ ID NO: 4 - DNA, 382 bp, synthetic construct
  misc_feature 1..382: note = Synthetic nucleic acid sequence
  source 1..382: mol_type = other DNA
acttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaacaactcgtggaggagagaaaagagacgagttgtgaagaagagagaggaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccag

SEQ ID NO: 5 - DNA, 489 bp, synthetic construct
  misc_feature 1..489: note = Synthetic nucleic acid sequence
  source 1..489: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaag

SEQ ID NO: 6 - DNA, 237 bp, synthetic construct
  misc_feature 1..237: note = Synthetic nucleic acid sequence
  source 1..237: mol_type = other DNA
gtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaggacctttga

SEQ ID NO: 7 - DNA, 382 bp, synthetic construct
  misc_feature 1..382: note = Synthetic nucleic acid sequence
  source 1..382: mol_type = other DNA
acttctaggcgcgccgcggaaaaccgcgggataccgcgatgattgcgagctagggagagagaggggaaagaaaagagaaagaggaggaggaaagaggggagagaggggagggaaaggagagaagggaggaagggaagaaagaaagaagaggaaaagaggggaggaggaggagaaaggagaaaaaaagaagggaagggagaaaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccag

SEQ ID NO: 8 - DNA, 301 bp, synthetic construct
  misc_feature 1..301: note = Synthetic nucleic acid sequence
  source 1..301: mol_type = other DNA
gtaagtgtcccgcggaacattattataacgttgctcgaagatatcagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgactctaga

SEQ ID NO: 9 - DNA, 326 bp, synthetic construct
  misc_feature 1..326: note = Synthetic nucleic acid sequence
  source 1..326: mol_type = other DNA
gctagcgtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctccgcggaacattattataacgttgctcgaatactaactggtacctcttcttttttttttgatatctgcag

SEQ ID NO: 10 - DNA, 278 bp, synthetic construct
  misc_feature 1..278: note = Synthetic nucleic acid sequence
  source 1..278: mol_type = other DNA
gttgcctttacttctggcgcgccaaaaggcgtgccagaagtaccgggctaataatgtttcgcggtcctcttaaatctgcctaaatacgtataaatttgatcgccctgaaaaagggcgatcaaagccctgaaaaagggcatacgtagccctgaaaaagggcaggcagagccctgaaaaagggcaagaggaccgcggaacattattagccgccaccatggacaggcgggttatgggacctgaaaatactaacaatcgattttttttccctttttttccag

SEQ ID NO: 11 - DNA, 190 bp, synthetic construct
  misc_feature 1..190: note = Synthetic nucleic acid sequence
  source 1..190: mol_type = other DNA
acttctaggcgcgccgcggaaaaccgcgggatatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccag

SEQ ID NO: 12 - DNA, 459 bp, synthetic construct
  misc_feature 1..459: note = Synthetic nucleic acid sequence
  source 1..459: mol_type = other DNA
gtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 13 - DNA, 382 bp, synthetic construct
  misc_feature 1..382: note = Synthetic nucleic acid sequence
  source 1..382: mol_type = other DNA
acttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaagaactcctggaggagagaaaagagaggagttctgaagaagagagaggaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccag

SEQ ID NO: 14 - DNA, 372 bp, synthetic construct
  misc_feature 1..372: note = Synthetic nucleic acid sequence
  source 1..372: mol_type = other DNA
gtaagtattaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 15 - DNA, 407 bp, synthetic construct
  misc_feature 1..407: note = Synthetic nucleic acid sequence
  source 1..407: mol_type = other DNA
gtaagtattagctctttctttccatgggttggcctcgccgcgtgaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 16 - DNA, 378 bp, synthetic construct
  misc_feature 1..378: note = Synthetic nucleic acid sequence
  source 1..378: mol_type = other DNA
gtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcggtaccaataaaatatctttattttcattacatctgtgtgttggttttttgtgtgactagt

SEQ ID NO: 17 - DNA, 309 bp, synthetic construct
  misc_feature 1..309: note = Synthetic nucleic acid sequence
  source 1..309: mol_type = other DNA
acttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaagaactcctggaggagagaaaagagaggagttctgaagaagagagaggaggctttagctcgcttcgttttcatcattattgcggccctgaaaaagggccgcttataacgttgctcgaattctactaacaatcgattttttttccctttttttccag

SEQ ID NO: 18 - DNA, 419 bp, synthetic construct
  misc_feature 1..419: note = Synthetic nucleic acid sequence
  source 1..419: mol_type = other DNA
atatccttttagggcagagtgaagagttaggaggaaggtggttgggagagggatttccaggccttaggacatcatgacagatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 19 - DNA, 275 bp, synthetic construct
  misc_feature 1..275: note = Synthetic nucleic acid sequence
  source 1..275: mol_type = other DNA
acttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaacaactcgtggaggagagaaaagagacgagttgtgaagaagagagaggaggctttagctcgcttcgttttcatcatttccaggccttaggacatcatgacatttttccttaactttgctcac

SEQ ID NO: 20 - DNA, 3975 bp, synthetic construct
  misc_feature 1..3975: note = Synthetic nucleic acid sequence
  source 1..3975: mol_type = other DNA
acttctaggcgcgccgccaccatgggattcgtgcggcagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcaaagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccagatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctcggtcccaaggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatctggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcgctacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccggatcctgcactacagcgacccctttatcctcttcctgttcctgctggccttctccaccgctacaatcatgctgtgttttctgctgtccaccttcttctccaaagcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcaaatgatgctgctggacgccgccgtgtatggactgctggcttggtatctggaccaggtgttccctgccgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagatcttcgagccctgcggcagacctgccgtggacagactgaacatcaccttctacgagaaccagattaccgcctttctgggccacaacggcgctggcaagacaaccacactgagcatcctcaccggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgctcgaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagaagcggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtgactagt

SEQ ID NO: 21 - DNA, 3611 bp, synthetic construct
  misc_feature 1..3611: note = Synthetic nucleic acid sequence
  source 1..3611: mol_type = other DNA
aggatttttgacctgctcgattgtccactgcgagcaggtcttttggagtcgggcgaggcggaagcccgactccttttggcatgcacgctagccgcgtcgtgcatgccttttatcgaattcgggttatgggaccagtgaaggctgagggaaggactgtcctgggactggacaggcgggttatgggacctgaaaatactaacaatcgattttttttccctttttttccaggaactgatttttctgctcccgaacaagaacttcaagcaccgggcctacgccagcctgttcagagagctggaagaaaccctggccgacctgggcctgtctagctttggcatcagcgacacccctctcgaagagatcttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcaaaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggccggggcctgatcgacctggctctgtctcaagccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatctaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggatccccaggctagaagaatgctctggaacgtgatcgtgtctatcatccgcgagggcagagctgtggtgctgacctctcactccatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctcgggccgctggtgcctctagacaagctcaggacggcgctcgggctgactacaaagaccatgacggtgattataaagatcatgacatcgactataaggatgacgatgacaaatgaggtaccaattcctcacctgcgatctcgagctttatttgtgaaatttgtgatgctattgctttatttgtaaccattataagctgcaataaacaagttaacaacaacaattgcattcattttatgtttcaggttcagggggaggtgtgggaggttttttaaactag

SEQ ID NO: 22 - DNA, 3975 bp, synthetic construct
  misc_feature 1..3975: note = Synthetic nucleic acid sequence
  source 1..3975: mol_type = other DNA
acttctaggcgcgccgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgccggaagcggagctactaacttcagcctgctgaagcaggctggagacgtggaggagaaccctggacctaggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtgactagt

SEQ ID NO: 23 - DNA, 3912 bp, synthetic construct
  misc_feature 1..3912: note = Synthetic nucleic acid sequence
  source 1..3912: mol_type = other DNA
aggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaggaccttggaagcggagctactaacttcagcctgctgaagcaggctggagacgtggaggagaaccctggacctatcacaaagaagcacacagcccacttctccaagaagggcgaagaggaaaacctggaaggcctgggcaatcagaccaagcagatcgtcgagaagtacgcctgcaccaccagaatcagccccaacacaagccagcagaacttcgtgacccagcggagcaaaagagccctgaagcagtttcggctgcccctggaagaaaccgagctggaaaagcggatcatcgtggacgacaccagcacacagtggtccaagaacatgaagcacttgacccctagcacactgacccagatcgactacaacgagaaagagaagggcgctatcacacagagcccactgagcgactgtctgaccagaagccacagcatccctcaggccaacagatcccctctgccaatcgccaaagtgtctagcttccccagcatcagacccatctacctgaccagagtgctgttccaggacaacagcagccatctgccagccgccagctaccggaagaaagatagcggcgtgcaagagtccagccactttctgcaaggcgctaagaagaacaatctgagcctggctattctgaccctggaaatgaccggcgatcagagagaagtcggctctctgggcaccagcgccacaaatagcgtgacctacaaaaaggtggaaaacaccgtgctgcctaagcctgacctgccaaagacaagcggcaaggtggaactgctgccaaaggtgcacatctaccagaaggacctgtttcctaccgagacaagcaacggctctcccggccatctggatctggtggaaggatctctgctgcagggaaccgagggcgccatcaagtggaacgaggccaatagacctggcaaggtgcccttcctgagagtggccacagagtctagcgccaagacaccctccaaactgctggatcccctggcctgggataaccactacggcactcagatccccaaagaggaatggaagtcccaagagaagtcccctgaaaagaccgccttcaagaagaaggacaccattctgtccctgaatgcctgcgagagcaaccacgccattgccgccatcaatgagggccagaacaagcccgagatcgaagtgacctgggccaagcagggaagaaccgagagactgtgctcccagaatcctcctgtgctgaagcggcaccagagagaaatcacccggaccacactgcagagcgaccaagaagagatcgattacgacgataccatcagcgtcgagatgaagaaagaagatttcgacatctacgacgaggacgagaatcagagccctcggagcttccagaagaaaaccaggcactactttattgccgccgtcgagcggctgtgggactacggaatgtctagctctcctcacgtgctgcggaatagagcccagtctggtagcgtgccccagttcaaaaaggtcgtgttccaagagttcaccgacggcagcttcacccagccactgtatagaggcgagctgaacgagcatctgggcctgctgggcccttatatcagagccgaagtggaagataacatcatggtcaccttccggaatcaggcctctcggccctacagcttctacagctccctgatctcctacgaagaggaccagagacagggcgcagagccccggaagaatttcgtgaagcccaacgagactaagacctacttttggaaggtgcagcaccatatggcccctacaaaggacgagttcgactgcaaagcctgggcctacttctccgatgtggacctcgagaaggatgtgcacagcggactcatcggcccactgcttgtgtgccacaccaacacactgaaccccgctcacggcagacaagtgacagtgcaagaattcgccctgtttttcaccatcttcgacgaaacgaagtcctggtacttcaccgaaaacatggaaagaaactgcagggccccttgcaacattcagatggaagatcccaccttcaaagagaactaccggttccacgccatcaacggctacatcatggacacactgcccggcctggttatggctcaggatcagagaatccggtggtatctgctgtccatgggctccaacgagaatatccactccatccacttctccggccacgtgttcaccgtgcggaaaaaagaagagtacaaaatggccctgtacaatctgtaccctggggtgttcgaaaccgttgagatgctgcctagcaaggccggaatttggagagtggaatgtctgattggagagcacctccacgccgggatgagcaccctgtttctggtgtactccaacaagtgtcagacccctctcggcatggcctctggccacattagagacttccagatcaccgccagcggacagtatggacagtgggcccctaaactggccagactgcactactccggcagcatcaatgcctggtccaccaaagagcctttcagctggatcaaagtggacctgctggctcccatgatcatccacggaatcaagacccagggcgccagacaaaagttcagcagcctgtacatcagccagttcatcatcatgtacagcctggacggaaagaagtggcagacctaccggggcaatagcaccggcacactgatggtgttcttcggcaacgtggactccagcggcattaagcacaacatcttcaaccctccaatcattgcccgatacatccggctgcaccccacacactacagcatcaggtctaccctgagaatggaactgatgggctgcgacctgaacagctgcagcatgcccctcggaatggaaagcaaggccatcagcgacgcccagatcacagcctctagctacttcaccaacatgttcgccacttggagcccctctaaggcccggcttcatctgcaaggcagaagcaacgcttggaggccccaagtgaacaaccccaaagaatggctgcaggtcgactttcagaaaaccatgaaagtgacaggcgtgaccacacagggcgtcaagtccctgctgacctctatgtacgtgaaagagtttctgatcagctccagccaggacggccaccagtggaccctgttcttccaaaacggcaaagtgaaagtgttccagggaaatcaggacagcttcacacccgtggtcaactccctggatcctccactgctgacaagatacctgcggattcaccctcagtcttgggtgcaccagattgccctgcggatggaagtgctgggctgtgaagctcaggacctctactgaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactag

SEQ ID NO: 24 - DNA, 3828 bp, synthetic construct
  misc_feature 1..3828: note = Synthetic nucleic acid sequence
  source 1..3828: mol_type = other DNA
acttctaggcgcgccgccaccatgtacccatacgatgttccagattacgcttatccttatgacgtgcctgactacgcctatccctacgacgtccccgactatgcagtgtacaagaaaaccctgttcgtggaattcaccgaccacctgttcaatatcgccaagcctcggcctccttggatgggactgctgggacctacaattcaggccgaggtgtacgacaccgtggtcatcaccctgaagaacatggccagccatcctgtgtctctgcacgccgtgggagtgtcttactggaaggcttctgagggcgccgagtacgacgatcagacaagccagagagagaaagaggacgacaaggttttccctggcggcagccacacctatgtctggcaagtcctgaaagaaaacggccctatggcctccgatcctctgtgcctgacatacagctacctgagccacgtggacctggtcaaggacctgaattctggcctgatcggagccctgctcgtgtgtagagaaggcagcctggccaaagagaaaacccagacactgcacaagttcatcctgctgttcgccgtgttcgacgagggcaagagctggcacagcgagacaaagaacagcctgatgcaggacagggatgccgcctctgctcgggcttggcctaagatgcacaccgtgaacggctacgtgaacagaagcctgcctggactgatcggctgccacagaaagtccgtgtactggcacgtgatcggcatgggcacaacacctgaggtgcacagcatctttctggaaggacacaccttcctcgtgcggaaccatagacaggccagcctggaaatcagccctatcaccttcctgaccgctcagaccctgctgatggatctgggccagtttctgctgttctgccacatcagctcccaccagcacgatggcatggaagcctacgtgaaggtggacagctgccccgaagaaccccagctgcggatgaagaacaacgaggaagccgaggactacgacgacgacctgaccgactctgagatggacgtcgtcagattcgacgacgataacagccccagcttcatccaaatcagaagcgtggccaagaagcaccccaagacctgggtgcactatatcgccgccgaggaagaggactgggattacgctcctctggtgctggcccctgacgacagaagctacaagagccagtacctgaacaacggccctcagcggatcggccggaagtataagaaagtgcggttcatggcctacaccgacgagacattcaagaccagagaggccatccagcacgagagcggaattctgggccctctgctgtatggcgaagtgggcgatacactgctgatcatcttcaagaaccaggccagcagaccctacaacatctaccctcacggcatcaccgatgtgcggcccctgtattctagaaggctgcccaagggcgtgaagcacctgaaggacttccctatcctgcctggcgagatcttcaagtacaagtggaccgtgaccgtggaagatggccccaccaagagcgaccctagatgtctgacacggtactacagcagcttcgtgaacatggaacgcgacctggccagcggcctgattggacctctgctgatctgctacaaagaaagcgtggaccagcggggcaaccagatcatgagcgacaagcggaacgtgatcctgtttagcgtgttcgatgagaaccggtcctggtatctgaccgagaacatccagcggtttctgcccaatcctgctggcgtgcagctggaagatcctgagttccaggcctccaacatcatgcactccatcaatggctatgtgttcgacagcctgcagctgagcgtgtgcctgcacgaagtggcctactggtacatcctgagcattggcgcccagaccgacttcctgtccgtgttcttttccggctacaccttcaagcacaagatggtgtacgaggataccctgacactgttcccattctccggcgagacagtgttcatgagcatggaaaaccccggcctgtggatcctgggctgtcacaacagcgacttccggaacagaggcatgacagccctgctgaaggtgtccagctgcgacaagaacaccggcgactactacgaggacagctatgaggacatcagcgcctacctgctgagcaagaacaatgccatcgagcccagaagcttcagccagaatagcagacacccctccaccagacagaagcagttcaacgccacaacaatccccgagaacgacatcgagaaaaccgatccttggtttgcccaccggacccctatgcctaagatccagaacgtgtcctccagcgatctgctgatgctcctgagacagagccctacacctcacggactgagcctgtccgatctgcaagaggccaaatacgaaaccttcagcgacgacccttctcctggcgccatcgacagcaacaatagcctgagcgagatgacccacttcagaccacagctgcaccacagcggcgacatggtgtttacacctgagagcggcctccagctgagactgaatgagaagctgggaaccaccgccgccaccgagctgaagaaactggacttcaaggtgtcctctaccagcaacaacctgatcagcacaatcccctccgacaacctggctgccggcaccgacaacacatcttctctgggcccacctagcatgcccgtgcactacgatagccagctggataccacactgttcggcaagaagtctagccctctgacagagtctggcggccctctgtctctgagcgaggaaaacaacgacagcaagctgctggaatccggcctgatgaacagccaagagtcctcctggggcaagaatgtgtccagcaccgagtccggcagactgttcaagggaaagagagcccacggacctgctctgctgaccaaggataacgccctgttcaaagtgtccatcagcctgctcaagaccaacaagacctccaacaactccgccaccaacagaaagacccacatcgacggccctagcctgctgatcgagaatagccctagcgtctggcagaatatcctggaaagcgacaccgagttcaagaaagtgacccctctgatccacgaccggatgctcatggacaagaacgccaccgctctgcggctgaaccacatgagcaacaagacaaccagcagcaagaatatggaaatggtgcagcagaagaaagagggccccattcctccagacgctcagaaccccgatatgagcttcttcaagatgctctttctgcccgagagcgcccggtggatccagagaacacacggcaagaactccctgaactccggccagggaccttctccaaagcagctggtttccctgggacctgagaagtccgtggaaggccagaacttcctgagcgaaaagaacaaagtggtcgtcggcaagggcgagttcaccaaggatgtgggcctgaaagagatggtctttcccagcagccggaacctgttcctgaccaacctggacaacctgcacgagaacaacacccacaatcaagagaagaagatccaagaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 25 - DNA, 3802 bp, synthetic construct
  misc_feature 1..3802: note = Synthetic nucleic acid sequence
  source 1..3802: mol_type = other DNA
aggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggaaatcgaaaagaaagagacactcatccaagagaacgtggtgctgcctcagatccacacagtgaccggcaccaagaactttatgaagaatctgttcctgctgagtacccggcagaatgtggaaggcagctacgacggcgcttatgcccctgtgctgcaagacttcagatccctgaacgactccaccaatcggacaaagaagcacacagcccacttctccaagaagggcgaagaggaaaacctggaaggcctgggcaatcagaccaagcagatcgtcgagaagtacgcctgcaccaccagaatcagccccaacacaagccagcagaacttcgtgacccagcggagcaaaagagccctgaagcagtttcggctgcccctggaagaaaccgagctggaaaagcggatcatcgtggacgacaccagcacacagtggtccaagaacatgaagcacttgacccctagcacactgacccagatcgactacaacgagaaagagaagggcgctatcacacagagcccactgagcgactgtctgaccagaagccacagcatccctcaggccaacagatcccctctgccaatcgccaaagtgtctagcttccccagcatcagacccatctacctgaccagagtgctgttccaggacaacagcagccatctgccagccgccagctaccggaagaaagatagcggcgtgcaagagtccagccactttctgcaaggcgctaagaagaacaatctgagcctggctattctgaccctggaaatgaccggcgatcagagagaagtcggctctctgggcaccagcgccacaaatagcgtgacctacaaaaaggtggaaaacaccgtgctgcctaagcctgacctgccaaagacaagcggcaaggtggaactgctgccaaaggtgcacatctaccagaaggacctgtttcctaccgagacaagcaacggctctcccggccatctggatctggtggaaggatctctgctgcagggaaccgagggcgccatcaagtggaacgaggccaatagacctggcaaggtgcccttcctgagagtggccacagagtctagcgccaagacaccctccaaactgctggatcccctggcctgggataaccactacggcactcagatccccaaagaggaatggaagtcccaagagaagtcccctgaaaagaccgccttcaagaagaaggacaccattctgtccctgaatgcctgcgagagcaaccacgccattgccgccatcaatgagggccagaacaagcccgagatcgaagtgacctgggccaagcagggaagaaccgagagactgtgctcccagaatcctcctgtgctgaagcggcaccagagagaaatcacccggaccacactgcagagcgaccaagaagagatcgattacgacgataccatcagcgtcgagatgaagaaagaagatttcgacatctacgacgaggacgagaatcagagccctcggagcttccagaagaaaaccaggcactactttattgccgccgtcgagcggctgtgggactacggaatgtctagctctcctcacgtgctgcggaatagagcccagtctggtagcgtgccccagttcaaaaaggtcgtgttccaagagttcaccgacggcagcttcacccagccactgtatagaggcgagctgaacgagcatctgggcctgctgggcccttatatcagagccgaagtggaagataacatcatggtcaccttccggaatcaggcctctcggccctacagcttctacagctccctgatctcctacgaagaggaccagagacagggcgcagagccccggaagaatttcgtgaagcccaacgagactaagacctacttttggaaggtgcagcaccatatggcccctacaaaggacgagttcgactgcaaagcctgggcctacttctccgatgtggacctcgagaaggatgtgcacagcggactcatcggcccactgcttgtgtgccacaccaacacactgaaccccgctcacggcagacaagtgacagtgcaagaattcgccctgtttttcaccatcttcgacgaaacgaagtcctggtacttcaccgaaaacatggaaagaaactgcagggccccttgcaacattcagatggaagatcccaccttcaaagagaactaccggttccacgccatcaacggctacatcatggacacactgcccggcctggttatggctcaggatcagagaatccggtggtatctgctgtccatgggctccaacgagaatatccactccatccacttctccggccacgtgttcaccgtgcggaaaaaagaagagtacaaaatggccctgtacaatctgtaccctggggtgttcgaaaccgttgagatgctgcctagcaaggccggaatttggagagtggaatgtctgattggagagcacctccacgccgggatgagcaccctgtttctggtgtactccaacaagtgtcagacccctctcggcatggcctctggccacattagagacttccagatcaccgccagcggacagtatggacagtgggcccctaaactggccagactgcactactccggcagcatcaatgcctggtccaccaaagagcctttcagctggatcaaagtggacctgctggctcccatgatcatccacggaatcaagacccagggcgccagacaaaagttcagcagcctgtacatcagccagttcatcatcatgtacagcctggacggaaagaagtggcagacctaccggggcaatagcaccggcacactgatggtgttcttcggcaacgtggactccagcggcattaagcacaacatcttcaaccctccaatcattgcccgatacatccggctgcaccccacacactacagcatcaggtctaccctgagaatggaactgatgggctgcgacctgaacagctgcagcatgcccctcggaatggaaagcaaggccatcagcgacgcccagatcacagcctctagctacttcaccaacatgttcgccacttggagcccctctaaggcccggcttcatctgcaaggcagaagcaacgcttggaggccccaagtgaacaaccccaaagaatggctgcaggtcgactttcagaaaaccatgaaagtgacaggcgtgaccacacagggcgtcaagtccctgctgacctctatgtacgtgaaagagtttctgatcagctccagccaggacggccaccagtggaccctgttcttccaaaacggcaaagtgaaagtgttccagggaaatcaggacagcttcacacccgtggtcaactccctggatcctccactgctgacaagatacctgcggattcaccctcagtcttgggtgcaccagattgccctgcggatggaagtgctgggctgtgaagctcaggacctctactgaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagt

SEQ ID NO: 26 - DNA, 10 bp, synthetic construct
  misc_feature 1..10: note = Synthetic nucleic acid sequence
  source 1..10: mol_type = other DNA
tggggggagg

SEQ ID NO: 27 - DNA, 10 bp, synthetic construct
  misc_feature 1..10: note = Synthetic nucleic acid sequence
  source 1..10: mol_type = other DNA
gtagtgaggg

SEQ ID NO: 28 - DNA, 10 bp, synthetic construct
  misc_feature 1..10: note = Synthetic nucleic acid sequence
  source 1..10: mol_type = other DNA
gttggtggtt

SEQ ID NO: 29 - DNA, 10 bp, synthetic construct
  misc_feature 1..10: note = Synthetic nucleic acid sequence
  source 1..10: mol_type = other DNA
agttgtggtt

SEQ ID NO: 30 - DNA, 10 bp, synthetic construct
  misc_feature 1..10: note = Synthetic nucleic acid sequence
  source 1..10: mol_type = other DNA
gtattgggtc

SEQ ID NO: 31 - DNA, 10 bp, synthetic construct
  misc_feature 1..10: note = Synthetic nucleic acid sequence
  source 1..10: mol_type = other DNA
agtgtgaggg

SEQ ID NO: 32 - DNA, 10 bp, synthetic construct
  misc_feature 1..10: note = Synthetic nucleic acid sequence
  source 1..10: mol_type = other DNA
gggtaatggg

SEQ ID NO: 33 - DNA, 10 bp, synthetic construct
  misc_feature 1..10: note = Synthetic nucleic acid sequence
  source 1..10: mol_type = other DNA
tcattggggt

SEQ ID NO: 34 - DNA, 10 bp, synthetic construct
  misc_feature 1..10: note = Synthetic nucleic acid sequence
  source 1..10: mol_type = other DNA
ggtgggggtc

SEQ ID NO: 35 - DNA, 10 bp, synthetic construct
  misc_feature 1..10: note = Synthetic nucleic acid sequence
  source 1..10: mol_type = other DNA
ggttttgttg

SEQ ID NO: 36 - DNA, 10 bp, synthetic construct
  misc_feature 1..10: note = Synthetic nucleic acid sequence
  source 1..10: mol_type = other DNA
tatactcccg

SEQ ID NO: 37 - DNA, 10 bp, synthetic construct
  misc_feature 1..10: note = Synthetic nucleic acid sequence
  source 1..10: mol_type = other DNA
gtattcgatc

SEQ ID NO: 38 - DNA, 10 bp, synthetic construct
  misc_feature 1..10: note = Synthetic nucleic acid sequence
  source 1..10: mol_type = other DNA
gtagttccct

SEQ ID NO: 39 - DNA, 10 bp, synthetic construct
  misc_feature 1..10: note = Synthetic nucleic acid sequence
  source 1..10: mol_type = other DNA
gttaatagta

SEQ ID NO: 40 - DNA, 10 bp, synthetic construct
  misc_feature 1..10: note = Synthetic nucleic acid sequence
  source 1..10: mol_type = other DNA
tgctggttag

SEQ ID NO: 41 - DNA, 10 bp, synthetic construct
  misc_feature 1..10: note = Synthetic nucleic acid sequence
  source 1..10: mol_type = other DNA
ataggtaacg

SEQ ID NO: 42 - DNA, 10 bp, synthetic construct
  misc_feature 1..10: note = Synthetic nucleic acid sequence
  source 1..10: mol_type = other DNA
tctgaattgc

SEQ ID NO: 43 - DNA, 10 bp, synthetic construct
  misc_feature 1..10: note = Synthetic nucleic acid sequence
  source 1..10: mol_type = other DNA
tctgggtttg

SEQ ID NO: 44 - DNA, 10 bp, synthetic construct
  misc_feature 1..10: note = Synthetic nucleic acid sequence
  source 1..10: mol_type = other DNA
cattctcttt

SEQ ID NO: 45 - DNA, 10 bp, synthetic construct
  misc_feature 1..10: note = Synthetic nucleic acid sequence
  source 1..10: mol_type = other DNA
gtattggtgt

SEQ ID NO: 46 - DNA, 10 bp, synthetic construct
  misc_feature 1..10: note = Synthetic nucleic acid sequence
  source 1..10: mol_type = other DNA
tttagatttg

SEQ ID NO: 47 - DNA, 10 bp, synthetic construct
  misc_feature 1..10: note = Synthetic nucleic acid sequence
  source 1..10: mol_type = other DNA
ataagtactg

SEQ ID NO: 48 - DNA, 10 bp, synthetic construct
  misc_feature 1..10: note = Synthetic nucleic acid sequence
  source 1..10: mol_type = other DNA
tagtctatta

SEQ ID NO: 49 - DNA, 10 bp, synthetic construct
  misc_feature 1..10: note = Synthetic nucleic acid sequence
  source 1..10: mol_type = other DNA
aggtattgca

SEQ ID NO: 50 - DNA, 10 bp, synthetic construct
  misc_feature 1..10: note = Synthetic nucleic acid sequence
  source 1..10: mol_type = other DNA
gtagattacg

SEQ ID NO: 51 - DNA, 10 bp, synthetic construct
  misc_feature 1..10: note = Synthetic nucleic acid sequence
  source 1..10: mol_type = other DNA
gggcgggtgc

SEQ ID NO: 52 - DNA, 10 bp, synthetic construct
  misc_feature 1..10: note = Synthetic nucleic acid sequence
  source 1..10: mol_type = other DNA
cgtttacaat

SEQ ID NO: 53 - DNA, 11 bp, synthetic construct
  misc_feature 1..11: note = Synthetic nucleic acid sequence
  source 1..11: mol_type = other DNA
gtacagggatg

SEQ ID NO: 54 - DNA, 10 bp, synthetic construct
  misc_feature 1..10: note = Synthetic nucleic acid sequence
  source 1..10: mol_type = other DNA
aatcagggga

SEQ ID NO: 55 - DNA, 10 bp, synthetic construct
  misc_feature 1..10: note = Synthetic nucleic acid sequence
  source 1..10: mol_type = other DNA
ggaggttttg

SEQ ID NO: 56 - DNA, 10 bp, synthetic construct
  misc_feature 1..10: note = Synthetic nucleic acid sequence
  source 1..10: mol_type = other DNA
gtattccctg

SEQ ID NO: 57 - DNA, 10 bp, synthetic construct
  misc_feature 1..10: note = Synthetic nucleic acid sequence
  source 1..10: mol_type = other DNA
tggtaagatc

SEQ ID NO: 58 - DNA, 10 bp, synthetic construct
  misc_feature 1..10: note = Synthetic nucleic acid sequence
  source 1..10: mol_type = other DNA
gtagttaagt

SEQ ID NO: 59 - DNA, 10 bp, synthetic construct
  misc_feature 1..10: note = Synthetic nucleic acid sequence
  source 1..10: mol_type = other DNA
gttggtttgg

SEQ ID NO: 60 - DNA, 10 bp, synthetic construct
  misc_feature 1..10: note = Synthetic nucleic acid sequence
  source 1..10: mol_type = other DNA
gtatttactt

SEQ ID NO: 61 - DNA, 10 bp, synthetic construct
  misc_feature 1..10: note = Synthetic nucleic acid sequence
  source 1..10: mol_type = other DNA
gtaacggggt

SEQ ID NO: 62 - DNA, 10 bp, synthetic construct
  misc_feature 1..10: note = Synthetic nucleic acid sequence
  source 1..10: mol_type = other DNA
tttttttctg

SEQ ID NO: 63 - DNA, 10 bp, synthetic construct
  misc_feature 1..10: note = Synthetic nucleic acid sequence
  source 1..10: mol_type = other DNA
ggggaaggga

SEQ ID NO: 64 - DNA, 10 bp, synthetic construct
  misc_feature 1..10: note = Synthetic nucleic acid sequence
  source 1..10: mol_type = other DNA
ttaccccggt

SEQ ID NO: 65 - DNA, 10 bp, synthetic construct
  misc_feature 1..10: note = Synthetic nucleic acid sequence
  source 1..10: mol_type = other DNA
gtattctatg

SEQ ID NO: 66 - DNA, 10 bp, synthetic construct
  misc_feature 1..10: note = Synthetic nucleic acid sequence
  source 1..10: mol_type = other DNA
aggtattgtg

SEQ ID NO: 67 - DNA, 10 bp, synthetic construct
  misc_feature 1..10: note = Synthetic nucleic acid sequence
  source 1..10: mol_type = other DNA
tttggggggg

SEQ ID NO: 68 - DNA, 10 bp, synthetic construct
  misc_feature 1..10: note = Synthetic nucleic acid sequence
  source 1..10: mol_type = other DNA
gttgttagcg

SEQ ID NO: 69 - DNA, 10 bp, synthetic construct
  misc_feature 1..10: note = Synthetic nucleic acid sequence
  source 1..10: mol_type = other DNA
ggtagttggg

SEQ ID NO: 70 - DNA, 10 bp, synthetic construct
  misc_feature 1..10: note = Synthetic nucleic acid sequence
  source 1..10: mol_type = other DNA
ctaagtactg

SEQ ID NO: 71 - DNA, 10 bp, synthetic construct
  misc_feature 1..10: note = Synthetic nucleic acid sequence
  source 1..10: mol_type = other DNA
aaccatcttc

SEQ ID NO: 72 - DNA, 10 bp, synthetic construct
  misc_feature 1..10: note = Synthetic nucleic acid sequence
  source 1..10: mol_type = other DNA
gtacctgggt

SEQ ID NO: 73 - DNA, 10 bp, synthetic construct
  misc_feature 1..10: note = Synthetic nucleic acid sequence
  source 1..10: mol_type = other DNA
gtatctcatt

SEQ ID NO: 74 - DNA, 10 bp, synthetic construct
  misc_feature 1..10: note = Synthetic nucleic acid sequence
  source 1..10: mol_type = other DNA
aaataaaatt

SEQ ID NO: 75 - DNA, 10 bp, synthetic construct
  misc_feature 1..10: note = Synthetic nucleic acid sequence
  source 1..10: mol_type = other DNA
ggtgggttat

SEQ ID NO: 76 - DNA, 10 bp, synthetic construct
  misc_feature 1..10: note = Synthetic nucleic acid sequence
  source 1..10: mol_type = other DNA
taagggaggg

SEQ ID NO: 77 - DNA, 10 bp, synthetic construct
  misc_feature 1..10: note = Synthetic nucleic acid sequence
  source 1..10: mol_type = other DNA
tatgggaggg

SEQ ID NO: 78 - DNA, 10 bp, synthetic construct
  misc_feature 1..10: note = Synthetic nucleic acid sequence
  source 1..10: mol_type = other DNA
gatgggaggg

SEQ ID NO: 79 - DNA, 10 bp, synthetic construct
  misc_feature 1..10: note = Synthetic nucleic acid sequence
  source 1..10: mol_type = other DNA
tggggggggt

SEQ ID NO: 80 - DNA, 10 bp, synthetic construct
  misc_feature 1..10: note = Synthetic nucleic acid sequence
  source 1..10: mol_type = other DNA
ggggaagggg

SEQ ID NO: 81 - DNA, 10 bp, synthetic construct
  misc_feature 1..10: note = Synthetic nucleic acid sequence
  source 1..10: mol_type = other DNA
tggtaagagg

SEQ ID NO: 82 - DNA, 10 bp, synthetic construct
  misc_feature 1..10: note = Synthetic nucleic acid sequence
  source 1..10: mol_type = other DNA
gggttagggt

SEQ ID NO: 83 - DNA, 10 bp, synthetic construct
  misc_feature 1..10: note = Synthetic nucleic acid sequence
  source 1..10: mol_type = other DNA
gtatcggggg

SEQ ID NO: 84 - DNA, 10 bp, synthetic construct
  misc_feature 1..10: note = Synthetic nucleic acid sequence
  source 1..10: mol_type = other DNA
ggttttgctg

SEQ ID NO: 85 - DNA, 10 bp, synthetic construct
  misc_feature 1..10: note = Synthetic nucleic acid sequence
  source 1..10: mol_type = other DNA
tgggggtgga

SEQ ID NO: 86 - DNA, 10 bp, synthetic construct
  misc_feature 1..10: note = Synthetic nucleic acid sequence
  source 1..10: mol_type = other DNA
acttttagag

SEQ ID NO: 87 - DNA, 10 bp, synthetic construct
  misc_feature 1..10: note = Synthetic nucleic acid sequence
  source 1..10: mol_type = other DNA
gtaacgggtt

SEQ ID NO: 88 - DNA, 10 bp, synthetic construct
  misc_feature 1..10: note = Synthetic nucleic acid sequence
  source 1..10: mol_type = other DNA
gtttggggga

SEQ ID NO: 89 - DNA, 10 bp, synthetic construct
  misc_feature 1..10: note = Synthetic nucleic acid sequence
  source 1..10: mol_type = other DNA
atttttagag

SEQ ID NO: 90 - DNA, 10 bp, synthetic construct
  misc_feature 1..10: note = Synthetic nucleic acid sequence
  source 1..10: mol_type = other DNA
ttaaagtagg

SEQ ID NO: 91 - DNA, 10 bp, synthetic construct
  misc_feature 1..10: note = Synthetic nucleic acid sequence
  source 1..10: mol_type = other DNA
gtattaatat

SEQ ID NO: 92 - DNA, 10 bp, synthetic construct
  misc_feature 1..10: note = Synthetic nucleic acid sequence
  source 1..10: mol_type = other DNA
ggtttgggtg

SEQ ID NO: 93 - DNA, 10 bp, synthetic construct
  misc_feature 1..10: note = Synthetic nucleic acid sequence
  source 1..10: mol_type = other DNA
tatgggaaag

SEQ ID NO: 94 - DNA, 10 bp, synthetic construct
  misc_feature 1..10: note = Synthetic nucleic acid sequence
  source 1..10: mol_type = other DNA
ggttgggagg

SEQ ID NO: 95 - DNA, 10 bp, synthetic construct
  misc_feature 1..10: note = Synthetic nucleic acid sequence
  source 1..10: mol_type = other DNA
gtatttagtg

SEQ ID NO: 96 - DNA, 10 bp, synthetic construct
  misc_feature 1..10: note = Synthetic nucleic acid sequence
  source 1..10: mol_type = other DNA
gagttaaatg

SEQ ID NO: 97 - DNA, 10 bp, synthetic construct
  misc_feature 1..10: note = Synthetic nucleic acid sequence
  source 1..10: mol_type = other DNA
ttgtaagttg

SEQ ID NO: 98 - DNA, 10 bp, synthetic construct
  misc_feature 1..10: note = Synthetic nucleic acid sequence
  source 1..10: mol_type = other DNA
tgggggtagg

SEQ ID NO: 99 - DNA, 10 bp, synthetic construct
  misc_feature 1..10: note = Synthetic nucleic acid sequence
  source 1..10: mol_type = other DNA
gttcttaggg

SEQ ID NO: 100 - DNA, 10 bp, synthetic construct
  misc_feature 1..10: note = Synthetic nucleic acid sequence
  source 1..10: mol_type = other DNA
gtattctaag

SEQ ID NO: 101 - DNA, 10 bp, synthetic construct
  misc_feature 1..10: note = Synthetic nucleic acid sequence
  source 1..10: mol_type = other DNA
ggaggttttg

SEQ ID NO: 102 - DNA, 10 bp, synthetic construct
  misc_feature 1..10: note = Synthetic nucleic acid sequence
  source 1..10: mol_type = other DNA
agaatatgta

SEQ ID NO: 103 - DNA, 10 bp, synthetic construct
  misc_feature 1..10: note = Synthetic nucleic acid sequence
  source 1..10: mol_type = other DNA
atctttcggg

SEQ ID NO: 104 - DNA, 10 bp, synthetic construct
  misc_feature 1..10: note = Synthetic nucleic acid sequence
  source 1..10: mol_type = other DNA
ttgcattgaa

SEQ ID NO: 105 - DNA, 10 bp, synthetic construct
  misc_feature 1..10: note = Synthetic nucleic acid sequence
  source 1..10: mol_type = other DNA
ggtgggattt

SEQ ID NO: 106 - DNA, 10 bp, synthetic construct
  misc_feature 1..10: note = Synthetic nucleic acid sequence
  source 1..10: mol_type = other DNA
tttatctaat

SEQ ID NO: 107 - DNA, 10 bp, synthetic construct
  misc_feature 1..10: note = Synthetic nucleic acid sequence
  source 1..10: mol_type = other DNA
gcgggtggtg

SEQ ID NO: 108 - DNA, 10 bp, synthetic construct
  misc_feature 1..10: note = Synthetic nucleic acid sequence
  source 1..10: mol_type = other DNA
ggtttagata

SEQ ID NO: 109 - DNA, 10 bp, synthetic construct
  misc_feature 1..10: note = Synthetic nucleic acid sequence
  source 1..10: mol_type = other DNA
tttatgcgtt

SEQ ID NO: 110 - DNA, 10 bp, synthetic construct
  misc_feature 1..10: note = Synthetic nucleic acid sequence
  source 1..10: mol_type = other DNA
tgggtaaggc

SEQ ID NO: 111 - DNA, 10 bp, synthetic construct
  misc_feature 1..10: note = Synthetic nucleic acid sequence
  source 1..10: mol_type = other DNA
gggggtggtc

SEQ ID NO: 112 - DNA, 10 bp, synthetic construct
  misc_feature 1..10: note = Synthetic nucleic acid sequence
  source 1..10: mol_type = other DNA
gtagtatatt

SEQ ID NO: 113 - DNA, 10 bp, synthetic construct
  misc_feature 1..10: note = Synthetic nucleic acid sequence
  source 1..10: mol_type = other DNA
ggaggtattt

SEQ ID NO: 114 - DNA, 10 bp, synthetic construct
  misc_feature 1..10: note = Synthetic nucleic acid sequence
  source 1..10: mol_type = other DNA
gtattgtaag

SEQ ID NO: 115 - DNA, 10 bp, synthetic construct
  misc_feature 1..10: note = Synthetic nucleic acid sequence
  source 1..10: mol_type = other DNA
tttacgggag

SEQ ID NO: 116 - DNA, 10 bp, synthetic construct
  misc_feature 1..10: note = Synthetic nucleic acid sequence
  source 1..10: mol_type = other DNA
tagttctggg

SEQ ID NO: 117 - DNA, 10 bp, synthetic construct
  misc_feature 1..10: note = Synthetic nucleic acid sequence
  source 1..10: mol_type = other DNA
ccacgtctat

SEQ ID NO: 118 - DNA, 10 bp, synthetic construct
  misc_feature 1..10: note = Synthetic nucleic acid sequence
  source 1..10: mol_type = other DNA
agtgggtagg

SEQ ID NO: 119 - DNA, 10 bp, synthetic construct
  misc_feature 1..10: note = Synthetic nucleic acid sequence
  source 1..10: mol_type = other DNA
caatttttac

SEQ ID NO: 120 - DNA, 10 bp, synthetic construct
  misc_feature 1..10: note = Synthetic nucleic acid sequence
  source 1..10: mol_type = other DNA
ggtctggggg

SEQ ID NO: 121 - DNA, 10 bp, synthetic construct
  misc_feature 1..10: note = Synthetic nucleic acid sequence
  source 1..10: mol_type = other DNA
atcaagattg

SEQ ID NO: 122 - DNA, 10 bp, synthetic construct
  misc_feature 1..10: note = Synthetic nucleic acid sequence
  source 1..10: mol_type = other DNA
gttagctaaa

SEQ ID NO: 123 - DNA, 10 bp, synthetic construct
  misc_feature 1..10: note = Synthetic nucleic acid sequence
  source 1..10: mol_type = other DNA
agtgtggggt

SEQ ID NO: 124 - DNA, 10 bp, synthetic construct
  misc_feature 1..10: note = Synthetic nucleic acid sequence
  source 1..10: mol_type = other DNA
ggtatgtggg

SEQ ID NO: 125 - DNA, 10 bp, synthetic construct
  misc_feature 1..10: note = Synthetic nucleic acid sequence
  source 1..10: mol_type = other DNA
gtagtgtggg

SEQ ID NO: 126 - DNA, 10 bp, synthetic construct
  misc_feature 1..10: note = Synthetic nucleic acid sequence
  source 1..10: mol_type = other DNA
aggaggtgtt

SEQ ID NO: 127 - DNA, 10 bp, synthetic construct
  misc_feature 1..10: note = Synthetic nucleic acid sequence
  source 1..10: mol_type = other DNA
gttggtaggt

SEQ ID NO: 128 - DNA, 10 bp, synthetic construct
  misc_feature 1..10: note = Synthetic nucleic acid sequence
  source 1..10: mol_type = other DNA
gtaggtggtt

SEQ ID NO: 129 - DNA, 10 bp, synthetic construct
  misc_feature 1..10: note = Synthetic nucleic acid sequence
  source 1..10: mol_type = other DNA
aggtgttggt

SEQ ID NO: 130 - DNA, 10 bp, synthetic construct
  misc_feature 1..10: note = Synthetic nucleic acid sequence
  source 1..10: mol_type = other DNA
tatggttgtg

SEQ ID NO: 131 - DNA, 10 bp, synthetic construct
  misc_feature 1..10: note = Synthetic nucleic acid sequence
  source 1..10: mol_type = other DNA
ttaggttagt

SEQ ID NO: 132 - DNA, 10 bp, synthetic construct
  misc_feature 1..10: note = Synthetic nucleic acid sequence
  source 1..10: mol_type = other DNA
gattggagtt

SEQ ID NO: 133 - DNA, 10 bp, synthetic construct
  misc_feature 1..10: note = Synthetic nucleic acid sequence
  source 1..10: mol_type = other DNA
gtagagtgga

SEQ ID NO: 134 - RNA, 24 nt, synthetic construct
  misc_feature 1..24: note = Synthetic nucleic acid sequence
  source 1..24: mol_type = other RNA
ctctttcttttccatgggttggct

SEQ ID NO: 135 - RNA, 24 nt, synthetic construct
  misc_feature 1..24: note = Synthetic nucleic acid sequence
  source 1..24: mol_type = other RNA
ggctgagggaaggactgtcctggg

SEQ ID NO: 136 - RNA, 13 nt, synthetic construct
  misc_feature 1..13: note = Synthetic nucleic acid sequence
  source 1..13: mol_type = other RNA
gggttatgggacc

SEQ ID NO: 137 - RNA, 12 nt, synthetic construct
  misc_feature 1..12: note = Synthetic nucleic acid sequence
  source 1..12: mol_type = other RNA
atatccttttta

SEQ ID NO: 138 - RNA, 12 nt, synthetic construct
  misc_feature 1..12: note = Synthetic nucleic acid sequence
  source 1..12: mol_type = other RNA
gtatccttttta

SEQ ID NO: 139 - RNA, 33 nt, synthetic construct
  misc_feature 1..33: note = Synthetic nucleic acid sequence
  source 1..33: mol_type = other RNA
aggcttcggagcaaggaggcagctccgaagcct

SEQ ID NO: 140 - RNA, 33 nt, synthetic construct
  misc_feature 1..33: note = Synthetic nucleic acid sequence
  source 1..33: mol_type = other RNA
aggcttcggagcaagcctccagctccgaagcct

SEQ ID NO: 141 - RNA, 29 nt, synthetic construct
  misc_feature 1..29: note = Synthetic nucleic acid sequence
  source 1..29: mol_type = other RNA
gtcgaggccgagcgggcaaaggcctcgac

SEQ ID NO: 142 - RNA, 29 nt, synthetic construct
  misc_feature 1..29: note = Synthetic nucleic acid sequence
  source 1..29: mol_type = other RNA
gtcgaggccgagcccgcaaaggcctcgac

SEQ ID NO: 143 - skipped

SEQ ID NO: 144 - RNA, 12 nt, synthetic construct
  misc_feature 1..12: note = Synthetic nucleic acid sequence
  source 1..12: mol_type = other RNA
ttttccttaact

SEQ ID NO: 145 - DNA, 1305 bp, synthetic construct
  misc_feature 1..1305: note = Synthetic nucleic acid sequence
  source 1..1305: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggtaagtattagctctttctttccatgggttggcctcgccgcgtgggctgagggaaggactgtcctgggactggacaggcgggttatgggacctgaaaagcggccctgaaaaagggccgcgatctgtagaaagcgagctagtgccggacagttagaggaaaaggggaagaactgtccgaaaaaagggggggaagacagtgactagaaagggaagggagaagtcactgtagaggggaaggaaaaggctagctagaggagaaggaaagaggctagctagcagaggagaaggaaaggcgccagcagttcggtgctatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 146 - DNA, 1543 bp, synthetic construct
  misc_feature 1..1543: note = Synthetic nucleic acid sequence
  source 1..1543: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgcggaaaaccgcgggatagcaccgaactgctggcgcctttccttctcctctgctagctagcctctttccttctcctctagctagccttttccttcccctctacagtgacttctcccttccctttctagtcactgtcttcccccccttttttcggacagttcttccccttttcctctaactgtccggcactagctcgctttctacagatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtattagctctttctttccatgggttggcctcgccgcgtgggctgagggaaggactgtcctgggactggacaggcgggttatgggacctgaaaagcggccctgaaaaagggccgcagcgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtgctaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 147 - DNA, 1571 bp, synthetic construct
  misc_feature 1..1571: note = Synthetic nucleic acid sequence
  source 1..1571: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccaccatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 148 - DNA, 1765 bp, synthetic construct
  misc_feature 1..1765: note = Synthetic nucleic acid sequence
  source 1..1765: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaagaactcctggaggagagaaaagagaggagttctgaagaagagagaggaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagt

SEQ ID NO: 149 - RNA, 10 nt, synthetic construct
  misc_feature 1..10: note = Synthetic nucleic acid sequence
  source 1..10: mol_type = other RNA
aaagaaggaa

SEQ ID NO: 150 - RNA, 12 nt, synthetic construct
  misc_feature 1..12: note = Synthetic nucleic acid sequence
  source 1..12: mol_type = other RNA
ctttcttttctt

SEQ ID NO: 151-153 - skipped (3 entries)

SEQ ID NO: 154 - DNA, 12 bp, synthetic construct
  misc_feature 1..12: note = Synthetic nucleic acid sequence
  source 1..12: mol_type = other DNA
gccgccaccatg